(19) United States
(12) Patent Application Publication
(10) Pub. No.: US 2014/0287447 A1
(43) Pub. Date: Sep. 25, 2014

(54) Ultrasensitive methodology for quantifying the kinase catalytic activity of any protein kinase in biological/clinical samples or recombinant/purified proteins using near-infrared-fluorescence (NIRF)-labeled, kinase-selective peptide substrates and a combination of kinase-selective inhibitors to define individual kinase activity

(71) Applicants: Ronald R. Fiscus, Las Vegas, NV (US); Benjamin F.B. Constantino, Las Vegas, NV (US); Mary G. Johlfs, Henderson, NV (US); Janica C. Wong, Sunnyvale, CA (US)

(72) Inventors: Ronald R. Fiscus, Las Vegas, NV (US); Benjamin F.B. Constantino, Las Vegas, NV (US); Mary G. Johlfs, Henderson, NV (US); Janica C. Wong, Sunnyvale, CA (US)

(21) Appl. No.: 14/213,965

(22) Filed: Mar. 14, 2014

Related U.S. Application Data
(60) Provisional application No. 61/785,017, filed on Mar. 14, 2013.

(51) Int. Cl. C12Q 1/48

(52) U.S. Cl. CPC C12Q 1/485
USPC 435/15; 530/329

(57) ABSTRACT

A non-radioactive, ultrasensitive methodology for the quantification of protein kinase catalytic activity of any protein kinase in, for example, biological/clinical samples or recombinant/purified proteins, based on using near-infrared-fluorescence (NIRF)-labeled peptide substrates that are selective for individual protein kinases and using a combination of kinase-selective inhibitors to define the catalytic activity of individual protein kinases, including but not limited to, a substrate for phosphorylation by a protein kinase comprising a core peptide having the formula: (N-terminus)-Arginine-Lysine-Arginine-Serine-Arginine-Lysine-Glutamic-acid-(C-terminus); and an indicator component covalently bonded to the core peptide.

### CROSS-REFERENCE TO RELATED APPLICATIONS

This application claims the benefit of U.S. Provisional Patent Application No. 61/785,017, which was filed on Mar. 14, 2013, the disclosure of which is incorporated herein by reference.

### FIELD OF THE DISCLOSURE

The present disclosure generally relates to an ultrasensitive methodology for quantifying the kinase catalytic activity of any protein kinase in biological/clinical samples or recombinant/purified proteins using near-infrared-fluorescence (NIRF)-labeled, kinase-selective peptide substrates and a combination of kinase-selective inhibitors to define individual kinase activity.

### DESCRIPTION OF THE RELATED ART

Protein kinases are key effector proteins with diverse cellular functions. A number of well documented disease states arise when protein kinase activity is altered. Therefore, the ability to accurately measure protein kinase catalytic activity has played a role in the development of effective pharmacological treatments that target these enzymes as new therapeutic agents for treating many different types of diseases. Traditional kinase assays measure the transfer of 32P from γ-32P ATP or 33P from γ-33P ATP to a target peptide or protein substrate, followed by separating the phosphorylated product from the radioactive ATP and then quantifying the peptide/protein phosphorylation level by scintillation counting. This technique, while efficient and highly sensitive, not only poses significant intrinsic disadvantages, including hazardous material handling, but often times also lacks kinase specificity, because the peptide and protein substrates are often phosphorylated by multiple protein kinases.

Moreover, in almost all prior techniques used to measure protein kinase activity, long incubation times (30 minutes up to 2 hours) are common practice and these reactions need various exogenous components, such as protease inhibitors and phosphatase inhibitors, added to them to preserve the integrity of the kinase activity. Specifically, prior techniques add a protease inhibitor cocktail to prevent protein degradation and phosphatase inhibitors to prevent dephosphorylation of the substrate during the reaction incubation.

Currently, the methodologies for measuring Protein kinase C (PKC) and protein kinase G (PKG) catalytic activities are based on the use of radioactivity (the “Gold standard” for protein kinase assays) or on optical absorbance or visible-light-fluorescence, which have much lower sensitivities and are thus not practical for measuring the protein kinase activities in small biological and clinical samples. A new methodology is needed that is both sensitive (equal in sensitivity to the radioactive assays) and specific for PKC and PKG catalytic activity that can be used for analyzing biological/clinical samples for basic science research, translational medical research, pharmaceutical sciences research and clinical research, including the diagnosing of diseases using protein kinase catalytic activity as biomarkers and for the development of new therapeutic agents and testing the effectiveness in human and animal patients.

Even further, in some radioactive kinase assays, low, unphysiological levels of ATP can lead to a dramatic over-estimation (by 100-1000 fold) of the potency of ATP-competitive kinase inhibitors, thus resulting in inaccurate estimations of the IC50 values. This major problem with the currently-used kinase assays contributes to the inaccuracy of early drug development and the lack of success of the kinase inhibitors when introduced into clinical trials. The kinase inhibitors being developed are simply not as potent or as effective as would be predicted based on the kinase assays used in the early stages of development. New methodology based on using physiological levels of ATP are needed for accurate measurement of PKC and PKG catalytic activity and the IC50 values of ATP-competing drugs and for improving the chances of success when these new agents are tested for effectiveness in clinical trials. Accordingly, there remains opportunity for improvement.

### SUMMARY OF THE DISCLOSURE

Some embodiments of the invention are directed to a substrate for phosphorylation by a protein kinase comprising a core peptide having the Formula: (N-terminus)-Arginine-Lysine-Arginine-Serine-Arginine-Lysine-Glutamic-acid-(C-terminus), and an indicator component covalently bonded to the core peptide.

In another embodiment, the disclosure provides a non-radioactive, ultrasensitive methodology for the quantification of protein kinase catalytic activity of any protein kinase, e.g. in biological/clinical samples or recombinant/purified proteins, based on using near-infrared-fluorescence (NIRF)-labeled peptide substrates that are selective for individual protein kinases and using a combination of kinase-selective inhibitors to define the catalytic activity of individual protein kinases.

### BRIEF DESCRIPTION OF THE DRAWINGS

Other advantages of the present disclosure will be readily appreciated, as the same becomes better understood by reference to the following detailed description when considered in connection with the accompanying drawings wherein:

Table 1 is a list of many protein kinases in humans and mice.

Table 2 is a list of NIRF dyes that can be conjugated to protein kinase substrate peptides.

Table 3 is a list of the constituents in the radioactive-free NIRF-based kinase reaction mixture.

Table 4 is a list of the constituents in the cell homogenization buffer.

Table 5 is the disclosure protocol for the radioactive-free protein kinase reaction using NIRF-histone-H2B(29-35).

Table 6 is a partial list of protein kinase inhibitors that can be used as added components in the instant kinase assay to define which protein kinases are contributing to the kinase catalytic activity.

Table 7 is a partial list of protein kinase substrate peptides that can be chemically conjugated to NIRF dyes and used to measure specific protein kinase activity using the kinase assay of this disclosure.

FIG. 1 is a schematic of NIRF-Histone-H2B(29-35), a near-infrared-fluorescence-labeled peptide specific for simultaneously measuring the catalytic activity of PKG and PKC isoforms.

FIG. 2 is a list of the PKC isoforms that can selectively phosphorylate NIRF-Histone-H2B(29-35) (i.e. use it as an effective substrate) and shows a radioisotope (³³P) assay designed to evaluate the effectiveness of the NIRF-histone-H2B(29-35) peptide as a substrate for the 12 most common isoforms of PKC and for PKG-Iα (the most common isoform of PKG). The graph shows the eight of the PKC isoforms and PKG-Iα are able to effectively phosphorylate this novel NIRF-labeled peptide.

FIG. 3 shows the experimental data that show the specificity of NIRF-Histone-H2B(29-35) as a substrate for PKC and PKG, but not for other related serine/threonine-protein kinases. More specifically, FIG. 3 shows seven protein kinases were assayed at a concentration of 50 nM each for 2 minutes at 30° C. using NIRF-Histone-H2B(29-35) as substrate and the methodology described herein (including separation of phosphorylated and unphosphorylated forms of the substrate by agarose gel electrophoresis). Percent phosphorylation was determined by dividing the integrated intensity values (calculated using the LI-COR Odyssey® Image Studio version 3.1) of phosphorylated NIRF-Histone-H2B(29-35) by the total pool of starting NIRF-Histone-H2B(29-35) and multiplying by 100 to get a percent. Both recombinant PKG-Iα (rPKG-Iα) and purified PKC (pPKC), but not the other related serine/threonine-protein kinases, were able to effectively phosphorylate NIRF-Histone-H2B(29-35) as a substrate. Recombinant PKA (rPKA) was only able to phosphorylate NIRF-Histone-H2B(29-35) at a level just above background, whereas the recombinant (catalytically-active) proteins of p70 S6K (r-p70 S6K), RSK2 (rRSK2), AKT1 (rAKT1), and AKT2 (rAKT2) showed no significant kinase catalytic activity using NIRF-Histone-H2B(29-35) as substrate.

FIG. 4 shows experimental data that determined the concentration of NIRF-Histone-H2B(29-35) for use in the disclosure. More specifically, FIG. 4 shows that to determine the Kₘ of the NIRF-Histone-H2B(29-35), the kinase reaction was performed using varying concentrations of nonphosphorylated NIRF-Histone-H2B(29-35) as a substrate for rPKG-Iα. Under the assay conditions, the apparent Kₘ value for the NIRF-Histone-H2B(29-35) is 15 μM.

FIG. 5 shows experimental data that determined the concentration of cGMP (allosteric activator of all PKG isoforms) to be used in the disclosure. More specifically, FIG. 5 shows different concentrations of the allosteric activator of PKG, cGMP, were added to the reaction mix to evaluate the best concentration of cGMP able to fully activate PKG-Iα. The bottom graph represents a curve fitting model with the basal activity of rPKG-Iα subtracted and only the kinase activity stimulated by cGMP shown. Using a curve fitting model (GraphPad, Prism), the K_{act} for cGMP=82.2 nM and the optimal concentration of cGMP for fully activating PKG-Ic as determined to be 10 μM which was used in all subsequent kinase reactions to determine total PKG catalytic activity in recombinant proteins and biological samples.

FIG. 6 shows experimental data that determined an optimum concentration of ATP for use in the disclosure. More specifically, FIG. 6 shows the concentration dependency of ATP for PKG-Iα catalytic activity was determined to be Kₘ=8.8 μM. However, to attain full activity of PKG-Iα, a concentration of 1 mM of ATP was used. All subsequent kinase reactions for measuring PKG catalytic activity contained ATP at 1 mM in order to have full activity. Also, 1 mM concentration of ATP can be used because it represents a typical physiological concentration within mammalian cells. Another advantage to using the higher concentration of ATP is that it minimizes the problem of decreases kinase activity caused by depletion of ATP during the kinase reaction.

FIG. 7 shows a flow of the radioactive-free NIRF-based protein kinase reaction protocol using NIRF-Histone-H2B(29-35) as substrate.

FIG. 8 is an illustration of four selective PKC inhibitors, AEB071, LY333-531, Gö 6976, and Gö 6983, used as a PKC-inhibitor cocktail to discriminate between PKG and PKC kinase activity in a complex mixture of protein kinase, such as biological or clinical samples.

FIGS. 9A-D represent “proof of principle” experiments, showing that PKC and PKG catalytic activities can be measured simultaneously and that each protein kinase can be separately defined by using a specially-formulated combination of selective PKC inhibitors (PKC-inhibitor cocktail). More specifically FIG. 9 shows purified PKCα, PKCβI, PKCβII, PKCγ (A) and PKCδ (B) activities are inhibited by >99.9% when exposed to a combination of selective PKC inhibitors (PKC-inhibitor cocktail), which included AEB071, LY333-531, Gö 6976, and Gö 6983 at 3 μM-1000 μM. Conversely, recombinant PKG-Iα activity is unaffected by exposure to the PKC-inhibitor cocktail (C). By using this PKC-inhibitor cocktail, an accurate determination of both PKC and PKG catalytic activities can be obtained, simultaneously, in samples that represent a complex mixture of multiple protein kinases, such as biological and clinical samples. This allows simultaneous determination of PKG-I kinase activity in MDA-MB-231 breast cancer cell lysates (D). The kinase activity inhibited by PKC-inhibitor cocktail represents PKC, whereas the remaining activity represents PKG. Note that exogenously-added cGMP (10 μM) does not cause increased kinase activity in the MDA-MB231 cell lysates, which reflect the hyperactivation (nearly-full activation) of PKG-Ic known to occur in cancer cells.

### DETAILED DESCRIPTION OF THE DISCLOSURE

The disclosure provides a non-radioactive, ultrasensitive methodology for the quantification of protein kinase catalytic activity of any protein kinase, e.g. in biological/clinical samples or recombinant/purified proteins, based on using near-infrared-fluorescence (NIRF)-labeled peptide substrates that are selective for individual protein kinases and using a combination of kinase-selective inhibitors to define the catalytic activity of individual protein kinases.

Protein kinases are involved in regulated the biological activity of both normal cells and cancer cells. There are more than 500 protein kinases encoded by the human genome and many of them are involved in the pathogenesis of a variety of diseases. Measurement of the catalytic activity of these kinases can provide valuable information for the diagnosis, prevention, and treatment of these diseases. Protein kinases represent a major target for newly developed pharmaceutical agents, which serve as either activators or inhibitors of the catalytic activity of these protein kinases.

Protein kinase C (PKC) and protein kinase G (PKG) represent two families of serine/threonine protein kinases that regulate important biological responses in both normal and cancerous mammalian cells, including cell survival, DNA synthesis/cell proliferation, cell attachment to extracellular matrix, cell migration and, in cancer cells, metastasis and invasion (forming secondary tumors). Targeting these two families of protein kinases by using pharmaceutical activators or inhibitors may represent a novel approach to preventing and treating numerous pathologies, including various forms of cancer, cardiovascular diseases (coronary artery diseases, hypertension, and stroke), diabetes mellitus (both Type 1 and Type 2 diabetes), neurological disorders (including Alzheimer's disease, diabetic neuropathies, mental depression, and Parkinson's disease), obesity, and many other pathological conditions.

PKC is recognized to play a role in the pathology of cancer, heart failure, myocardial infarction, pain and bipolar disorder. Furthermore, PKC is known to be involved in the pathogenesis and pathological complications of diabetes (especially the development of diabetic neuropathies.

PKG plays a role in the normal physiology of the cardiovascular system and abnormalities of this protein kinase are intimately involved in the pathogenesis of many cardiovascular diseases as well as erectile dysfunction (ED). Specifically, many of the therapeutic agents for treating hypertension and angina pectoris (chest pain during a heart attack), including the pharmaceutical agents nitroglycerin and other nitric oxide (NO) mimetics/NO donors (the NITRATES), work by elevating cyclic GMP (cGMP) levels and activating PKG kinase (catalytic) activity, which lowers blood pressure and reduces the stress on the heart, thus alleviating the chest pain. Furthermore, three major therapeutic agents for treating ED (i.e. Viagra®, Cialis® and Levitra®) work by synergistically enhancing the biological effects of endogenous NO, resulting in enhanced catalytic activity of PKG in the penis and the nerves innervating the penis, ultimately promoting penile erection.

PKG, at physiological levels of activity, can play a role in neural cells, mediating “neuroprotection”, i.e. protecting against the development of aging-related neural diseases, such as Alzheimer's disease and Parkinson's disease. Furthermore, PKG can exhibit catalytic activity in the proliferation, migration (invasion) and chemoresistance of various types of cancer cells, including breast cancer, lung cancer, mesothelioma, neuroblastoma, ovarian cancer, pancreatic cancer and prostate cancer cells. Moreover, PKG can exhibit catalytic activity within bone marrow-derived mesenchymal (stromal) stem cells in promoting cell survival, cell migration and cell proliferation.

Chemical agents that enhance PKG catalytic activity in neural cells can potentially become a new generation of therapeutic agents for treating aging-related neurological diseases (Alzheimer's disease and Parkinson's disease). Furthermore, selective targeting of the abnormal PKG catalytic activity in cancer cells can represent a new generation of anti-cancer therapies.

In vitro kinase assays that can accurately measure the catalytic activities of PKC and PKG are especially valuable in the diagnoses of diseases like cancer, diabetes and Alzheimer's and in the development of new therapeutic agents for effectively preventing and treating these diseases as well as many other diseases.

This disclosure also provides use of physiological levels of ATP (i.e. 1-10 mM) in kinase reactions, unlike with radioactive methods that typically require much lower/non-physiological levels of ATP, and use of shortened kinase-reaction times, if desired (e.g. 1 or 2 minutes, assuring measurements at the “initial velocity” of the kinase reaction, the true catalytic activity), thus typically resulting in a more accurate quantification of catalytic activity of the protein kinases and a more accurate determination of potency (IC50 values) when testing kinase inhibitors. This NIRF-based methodology has sensitivity similar to radioactive methodologies (but without the hazards of radioactivity). Furthermore, because NIRF fluorophores possess much lower background noise compared with visible fluorophores (typically >100-times lower background noise, giving >100-times better signal-to-noise ratio), especially when using biological/clinical samples that contain autofluorescence in the visible light range, the NIRF-based methodology can achieve a greater sensitivity compared to visible-fluorescence methods.

The disclosure further provides an example of this NIRF-based methodology utilizing a kinase-selective NIRF-peptide substrate and a combination of four kinase-selective inhibitors to accurately define individual protein kinase activities in complex mixtures of kinases, such as biological and clinical samples. Two clinically-important protein kinases, protein kinase C (PKC) and protein kinase G (PKG), are simultaneously quantified in biological/clinical samples (e.g. breast cancer cell lysates) containing a complex mixture of multiple protein kinases. For this example, a NIRF fluorophore, conjugated to a seven amino acid sequence (RKRSRKE) that is selectively phosphorylated by PKG and eight (out of twelve) isoforms of the PKC family of protein kinases, can be used. Some embodiments of the disclosure also include the addition of a combination of PKC-selective inhibitors (specifically, AEB071, LY333-531, Gö 6976, and Gö 6983, which inhibit selective isoforms of PKC) to accurately define the kinase catalytic activities of PKC and PKG, simultaneously, in biological/clinical samples. This methodology, using kinase-selective NIRF-labeled peptide substrates and kinase-selective inhibitors, along with physiological levels of ATP and shortened (1 or 2 minute) kinase reaction times, can be used for the accurate quantification of the catalytic activity of any protein kinase in biological and clinical samples (identifying new biomarkers for diseases or testing effectiveness of therapeutic agents in patients) and for accurately determining the potency/IC50 values of any protein kinase inhibitor being developed as a potential therapeutic agent.

This disclosure further provides a safe and sensitive alternative to currently-used radiometric assays for the quantification of the kinase enzymatic (catalytic) activity of all protein kinases in recombinant and purified proteins as well as biological and clinical samples by using near-infrared-fluorescence (NIRF)-labeled peptide substrates that are selectively phosphorylated by individual protein kinases, with the kinase reactions being carried out with a combination of kinase-selective inhibitors to accurately define the catalytic activity of individual protein kinases, and then to quantify and analyze the results by a NIRF imaging/quantification instrument.

In one embodiment of the disclosure, the protein kinase catalytic activities of two protein kinases, protein kinase C (PKC) and protein kinase G (PKG), are measured simultaneously and selectively in recombinant/purified protein samples as well as in biological/clinical samples (specifically, breast cancer cell lysates). NIRF-labeled peptide substrates that are selectively phosphorylated by PKG and certain isoforms of PKC are used and then the individual kinase activities of these two protein kinases are defined using a combination of four isoform-selective PKC inhibitors.

The technology of this disclosure can also be used to screen for protein kinase inhibitors that can be used as new therapeutic agents for treating many different types of diseases (cancer, inflammation, diabetes, cardiovascular diseases, Alzheimer's disease, Parkinson's disease, and many other diseases). Because of the methodology of this disclosure, which allows the use of physiological levels of ATP and the capability of using freshly-prepared biological/clinical samples as a source of “real”, clinically-relevant protein kinases (as opposed to using recombinant protein kinases than often do not function like protein kinases within mammalian cells or in biological/clinical samples), a more accurate determination of efficacy and potency (IC50 values) as kinase inhibitors can be attained.

Some embodiments of the disclosure aim to overcome the hazards and limitations of traditional kinase assays, among other things. In other embodiments, a rapid, highly sensitive, quantitative methodology that is a safer alternative to traditional radioactive assays for quantifying protein kinase catalytic activity of any protein kinase is described. In addition, some embodiments of the disclosure include a methodology for the simultaneous and selective quantification of catalytic activity of PKC and PKG in recombinant/purified proteins as well as in biological/clinical samples. Certain aspects of the disclosure can be modified using selective NIRF-labeled peptide/protein substrates to measure the kinase activity of all known protein kinases.

Some embodiments of the disclosure can be used for studying the molecular/cellular biology, the development of new therapies for various human and animal diseases and the discovery of new biomarkers for diagnosis and determine therapeutic effectiveness in human and animal diseases, including, but not limited to, the pathological complications thereof many diseases, such as cancers (brain cancer, breast cancer, colon cancer, leukemia, liver cancer, lung cancer, lymphoma, melanoma, mesothelioma, myeloma, ovarian cancer, pancreatic cancer, prostate cancer, renal cancer, and many other types of cancer), diabetes (both Type 1 and Type 2 diabetes mellitus), obesity, erectile dysfunction (ED), Alzheimer's disease, Parkinson's disease, mental depression, bipolar disorder, coronary artery disease, inflammation, systemic hypertension, pulmonary hypertension, stroke, and renal disease. Additionally, the disclosure provides highly adaptable for high throughput screening (HTS), the identification of drug leads, and drug discovery.

The disclosure does not require the use of specialized microtiter plates, indirect labeling using antibodies, or procedures that include a washing step. Additionally, the disclosure can accurately measure kinase catalytic activity without the need for the tedious procedure of excising agarose gel bands and subsequent agarose gel processing that is used to measure the amount of fluorescence in the gel slices. Furthermore, the methodology of this disclosure can use fluorophores in the near infrared range (i.e. NIRF labels), rather than visible fluorophores, which can give a much better signal-to-noise ratio (typically >100-times better), potentially improving the sensitivity and specificity of this NIRF-based methodology. This difference in signal-to-noise ratio can be important when analyzing biological and clinical samples that would have many chemicals, such as NADH, riboflavins and flavin coenzymes, that have intrinsic autofluorescence in the visible light range (but not in the near infrared range) and would thus give high background noise with biological/clinical samples.

Further embodiments of the disclosure include a combination of a near-infrared-fluorescently-labeled peptide (NIRF-Histone-H2B(29-35)) selective for PKG and some other the more common isoforms of PKC, a radioactive-free kinase reaction mix that contains physiological levels of ATP (i.e. 1 mM), a specially-formulated cell homogenization/lysis buffer for lysing cells or homogenizing tissue samples, a unique optimized protocol for discriminating between PKC and PKG kinase activity [using a combination of four isoform-selective PKC inhibitors for defining PKC and PKG catalytic activity in complex mixtures of protein kinases (e.g. biological and clinical samples)], use of an electrophoretic agarose gel unit, and use of a near-infrared-fluorescence imager to identify and quantify PKC and PKG kinase/catalytic activity in biological samples.

In even further embodiments, the disclosure utilizes the above constituents in an approach to replace existing hazardous radioactive techniques and as a better alternative to other methods based on ELISA or on the use of visible fluorescence (with potential of higher background noise). The methodology of this disclosure can be performed in labs that have a near-infrared-fluorescence imaging/quantification system (e.g. LI-COR's Odyssey® system, which is commonly found in biomedical research labs, pharmaceutical sciences labs, and clinical labs), without the need for special radioactive requirements, making this disclosure easily accessible to many basic science, translational medical research, and clinical laboratories.

This disclosure also provides a NIRF-Histone-H2B(29-35) peptide which can be used as an example of the value of this methodology for any protein kinase, is a selective substrate for PKG and certain commonly-studied isoforms of the PKC family of protein kinases. This peptide substrate may include a seven amino acid sequence (RKRSRKE) recognized by both PKG and certain PKC isoforms, but may not be recognized by other related protein kinases (e.g. protein kinase A, p70 S6 kinase, RSK-2, Akt1 and Akt2). Biological and clinical samples that contain PKG and certain PKC isoforms may show catalytic activity in the radioactive-free kinase mix, resulting in phosphorylation of the NIRF-Histone-H2B(29-35). Phosphorylation of NIRF-Histone-H2B(29-35) may change the charge and can be easily separated from a pool of nonphosphorylated NIRF-Histone-H2B(29-35) on a 1% agarose gel using a 300 volt power source common for electrophoresis units. The gel can be quantified on any gel imaging system equipped for near infrared detection, such as the LI-COR Odyssey® Imaging System.

As an alternative separation technique for separating the phosphorylated and nonphosphorylated NIRF-labeled peptide substrate at the end of the kinase reaction, capillary electrophoresis or microchip electrophoresis instruments can be used for separating and quantifying the kinase reaction, if these instruments were equipped for measuring NIRF labels.

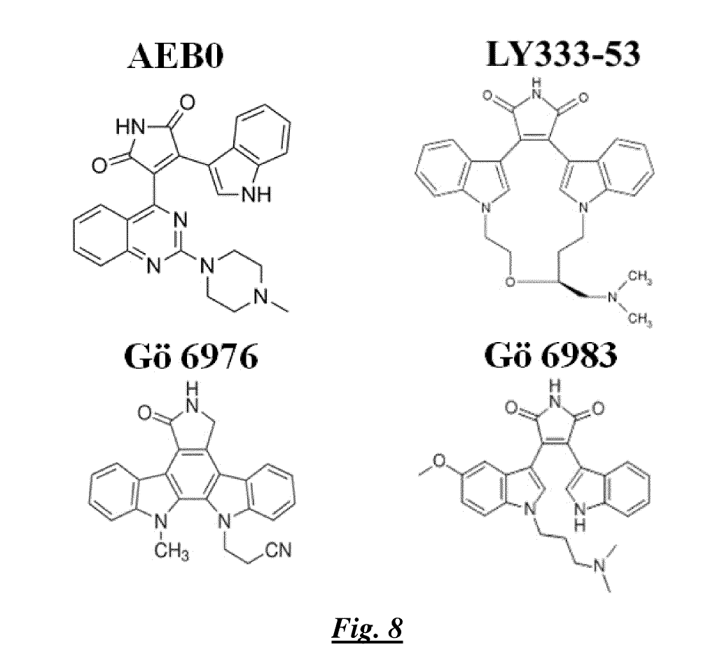

The instant methodology can include a compound or a combination of several compounds that inhibit certain proteins kinases, which can be important when analyzing kinase catalytic activity in a complex mixture of multiple protein kinases, such as in biological and clinical samples. Some embodiments of the disclosure give an example of the use of a combination of four isoform-selective PKC inhibitors, i.e. AEB071, LY333-531, Gö 6976, and Gö 6983 (FIG. 8), used to discriminate between PKC and PKG catalytic activity. Adding these compounds to the radioactive-free kinase reaction mix may only permit PKG phosphorylation of NIRF-Histone-H2B(29-35). A comparison of the kinase activity with and without the addition of these PKC inhibiting compounds may reflect the total amount of PKC activity and PKG activity in any biological/clinical sample. Additionally, the disclosure can support the addition of other kinase inhibitors to the radioactive-free kinase reaction mix; a partial collection of kinase inhibitors that can be used are listed in Table 6.

Additional advantages to some embodiments of the disclosure may include a short incubation time of 2 minutes and a reaction mix that can be devoid of certain added chemicals, such as protease inhibitors or phosphatase inhibitors, that can potentially interfere with or alter the true protein kinase activity of the samples. The 2 minute incubation time of the reaction allows for the preservation of the “initial velocity” (initial rate of reaction, the true measure of kinase catalytic activity). All other kinase assays that are commercially available or are provided as a serve typically use a much longer reaction time (usually 30-120 minutes), which may result in an erroneous quantification of the real catalytic activity of the protein kinases being examined. Although some recombinant protein kinases may be able to maintain a fairly linear reaction over this time, other samples, such as biological and clinical samples normally have a non-linear trend over this period of time. Typically, kinase reactions in a biological/clinical sample lose linearity within 3 to 5 minutes, thus measurements using reaction time greater than this would lead to erroneous estimations of kinase activity. The measurements taken at shorter time points, like 2 minutes, as in the disclosure, provide an accurate evaluation of true kinase kinetics in the biological/clinical samples, which is valuable information for developing pharmacological compounds to alter the function of target kinases. Shorter incubation times allow for a reaction mix that may lack exogenously added components (such as protease inhibitors and phosphatase inhibitors) that may have an adverse effect on the measurement of the kinase activity.

In still further embodiments, the reaction mixture excludes these components and maintain a more physiologically analogous reaction environment, especially by using a much shorter reaction incubation time period, thus avoiding loss of proteins because of proteolysis and potentially avoiding the dephosphorylation caused by phosphatases. This shorter reaction time also avoids the problem of ATP depletion that would occur in biological/clinical samples during the kinase reaction. Kinase reactions can also be conducted at reduced temperature (e.g. 0° C.) to avoid proteolytic loss of proteins, dephosphorylation of substrate and depletion of ATP.

In other embodiments, the composition of the NIRF-labeled peptide can be specifically designed for a target protein kinase or family of kinases. Therefore, the disclosure can be applied to all protein kinases and is well adapted for high throughput screening (HTS) methodologies, for the identification of novel drug compounds, for drug discovery, for the measurement of biomarkers of diseases, for the determination of effectiveness of therapies in clinical samples, among other things. A partial list of other peptides that can be used as kinase-selective substrates of individual protein kinases is listed in Table 7.

Some embodiments of the disclosure provide a unique radioactive-free protein kinase methodology, such that this protein kinase assay methodology is relevant to the fields of basic biology, basic biochemistry, biomedical sciences, pharmacology, pharmaceutical sciences, drug development, translational medical research, and clinical sciences, including discovery of new biomarkers of diseases and determining the effectiveness of therapeutic agents in humans and animals with diseases. In some embodiments of methodologies constructed according to the disclosure, such as the aforementioned methodology, the radioactive-free measurement of kinase activity of PKG and certain PKC isoforms is achieved using a near infrared-labeled peptide (NIRF-Histone-H2B(29-35)) selective for PKG and these PKC isoforms. In some embodiments of systems constructed according to the disclosure, such as the aforementioned system, the radioactive-free measurement of the kinase activity of all other kinases is achieved using a novel near infrared labeled peptide selective for any isoforms of any of the protein kinases. In other embodiments of systems constructed according to the disclosure, such as the aforementioned system, a unique radioactive-free kinase reaction mixture is used to achieve radioactive-free kinase measurements. In further embodiments of systems constructed according to the disclosure, such as the aforementioned system, a specially designed combination of multiple PKC inhibitors used to discriminate between PKC and PKG activity may be further included. In still other embodiments of systems constructed according to the disclosure, such as the aforementioned system, a unique protocol is followed to achieve radioactive-free kinase reaction measurements of PKC and PKG kinase (catalytic) activity. Alternatively, a specially formulated homogenizing buffer is used to achieve radioactive-free kinase measurements in biological samples. Alternatively, a specially formulated purified/recombinant protein buffer is used to achieve radioactive-free kinase measurements in purified/recombinant samples. Even further, the system may further include a near infrared scanner for scanning and quantification.

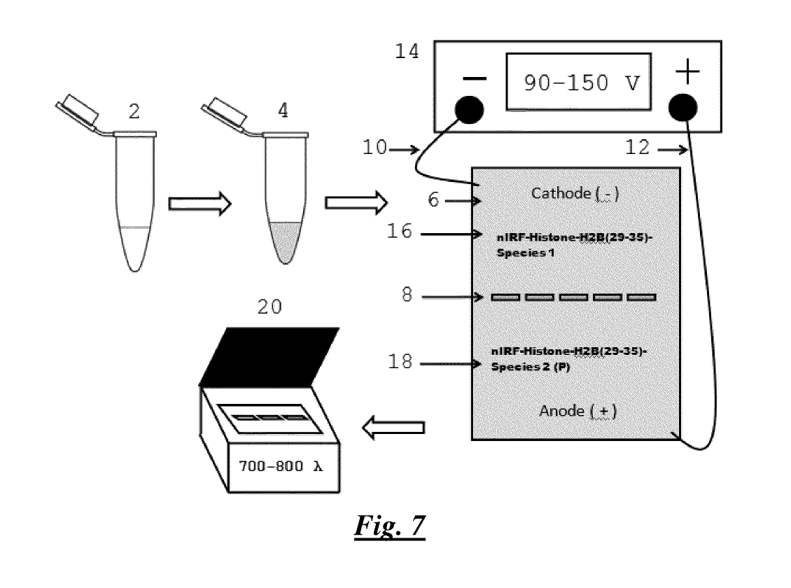

In one embodiment, such as set forth in FIG. 7, numeral 2 represents preparation of biological sample, numeral 4 represents a tube containing the Table 3 radioactive-free kinase reaction mixture; numeral 6 represents 1% Agarose gel, numeral 8 represents loading wells on agarose gel, numeral 10 represents a negative terminal, numeral 12 represents a positive terminal, numeral 14 represents a power source, numeral 16 represents a pool of non-phosphorylated NIRF-Histone-H2B(29-35) (Species 1), numeral 18 represents a pool of phosphorylated NIRF-Histone-H2B(29-35) (Species 2), and numeral 20 represents a near infrared imager able to scan at wavelengths: 700-800 nm.

In other embodiments, the assay of this disclosure is free of radioactivity. For example, near-infrared-fluorescence (NIRF)-labeled peptide substrates can be used which have a sensitivity that is equal to that of radioactive assays and is a safer alternative, which biomedical researchers, pharmaceutical/biotechnology companies, and clinical analysis laboratories are seeking.

In further embodiment, this disclosure provides technology that does not require a special handling permit, thereby making an assay highly accessible to all researchers and industries.

In some embodiments, and unlike assays based on radioactive isotopes, all components are easily disposable, with no special permits used.

In other embodiments, near-infrared-fluorescence (NIRF) is used as the label for measurement, providing a sensitivity that is much greater than obtained with visible-light-fluorescence or optical absorbance used by other non-radioactive protein kinase assays. NIRF-based assaying can provide a sensitivity that equals the previous “Gold standard” assays using radioactivity.

In still other embodiments, physiological concentrations of ATP [i.e. 1 millimolar (mM)] are used, compared with 0.001 to 0.03 mM (unphysiological) concentrations of ATP) used in most other kinase assays for measuring PKC and PKG catalytic activity. This can make a difference in determining the IC50 values for the kinase inhibitors that work by competing at the ATP binding site (representing most of the newly-developed kinase inhibitors for treating cancer).

In other embodiments, simultaneous measurement of PKC and PKG catalytic activity in biological samples is conducted. For example, in these embodiments, the present methodology allows simultaneous measurement of both PKC and PKG catalytic activity, defined by using highly selective kinase inhibitors within the assay. Also, the NIRF-labeled peptide substrate can also eliminate the potential interference by other protein kinases, which is a common (often unrecognized) problem in most other protein kinase assays. Substrates used in most other protein kinase assays can be phosphorylated by numerous other protein kinases (out of the 518 known protein kinases, based on the human genome), making it impractical to use the other kinase assays with biological and clinical samples that typically containing hundreds of different protein kinases. The present assay tends to be unique in being able to differentiate between the many different types of protein kinases in biological samples (tissue samples, blood samples) and clinical samples (biopsies, plasma, serum, whole blood, lymphatic fluid, cerebrospinal fluid (CSF), interstitial fluid, saliva, sweat or urine).

In other embodiments, the instant assay is well suited for high throughput screening, the identification of drug leads, and drug discovery methodologies. The design of some embodiments of the disclosure may be based on kinase-selective NIRF-labeled peptides and a combination of kinase-selective inhibitors and combined with short incubation times and reduced components to the reaction mixture make this assay a highly sensitive, simple, easy to use, and highly adaptable assay to screen a profile of kinase proteins in a high throughput manner. In one particular embodiment of the disclosure, the assay can be used to measure the activity of any protein kinase listed in Table 1, depending on the use of the kinase-selective peptide substrates and the right combination of kinase-selective inhibitors and is not limited to any kinase not listed in Table 1.

In further embodiments, this disclosure describes a simple, rapid, ultrasensitive protein kinase assay that provides a single system useful in diverse methodologies. Some embodiments of the disclosure are capable of accurately measuring kinase activity in a variety of important research settings, including, but not limited to, screening for new biomarkers for diseases and disease complications, diagnosis of diseases, prevention of diseases, and development of new therapies, including new pharmaceutical agents, new cell-based therapies (including stem-cell-based therapies).

In other embodiments, the methodology is based on utilization of P-32 radioactive phosphorous for measuring intracellular kinase activity of cAMP-dependent protein kinase (PKA) and cGMP-dependent protein kinase (PKG) in biological samples. Assays for both PKA and PKG can use full-length histone H2B as the protein substrate for measuring kinase activity. The specific site in histone H2B phosphorylated by PKG tends to be the serine-32 residue of this substrate and various peptides representing the amino acid sequence surrounding this site have been tested as potential substrates for PKG.

Although a seven-amino acid peptide representing the exact sequence of histone H2B phosphorylation by PKG (i.e. RKRSRKE) gives a somewhat slower catalytic rate, compared with an analog that had an alanine modification at lysine-34 position, the specificity between PKC and PKG is much better with the authentic sequence of histone H2B and thus this is used herein, in various non-limiting embodiments.

In further embodiments, the methodology of the present assay uses a NIRF-labeled peptide representing the authentic sequence [i.e. RKRSRKE, also called histone-H2B(29-35)] surrounding the PKG-catalyzed phosphorylation site in histone H2B, because of its greater selectivity for PKG. This NIRF-labeled peptide can also be an excellent substrate for PKC, but is not a substrate for related protein kinases, as shown in the data set forth herein. Both PKG and PKC effectively phosphorylate histone-H2B(29-35) which allows the protein kinase methodology of this disclosure and the simultaneous measurement of both PKC and PKG in single biological/clinical samples.

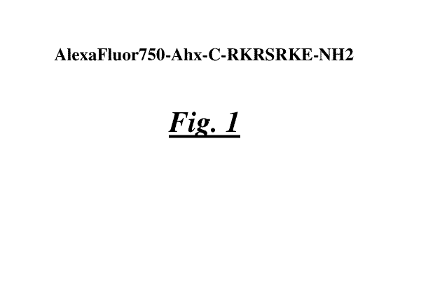

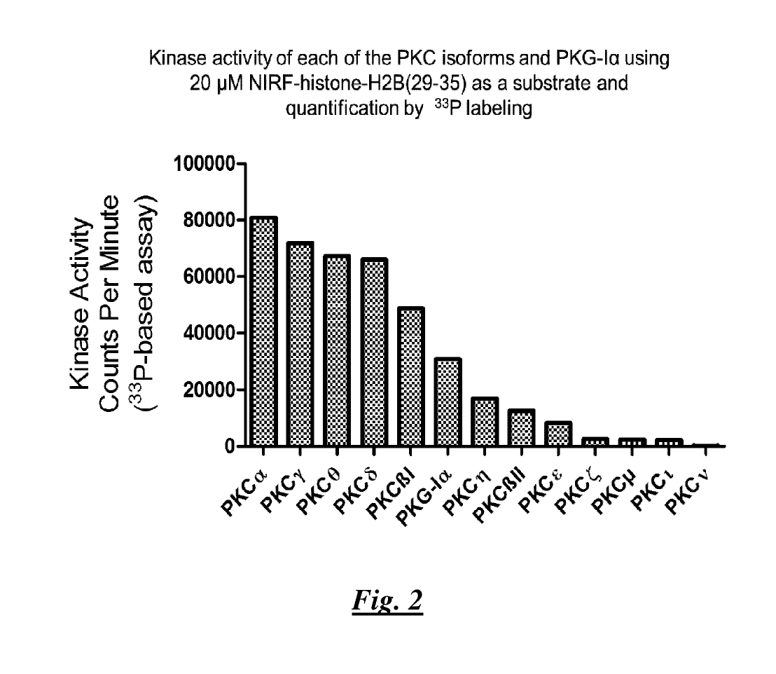

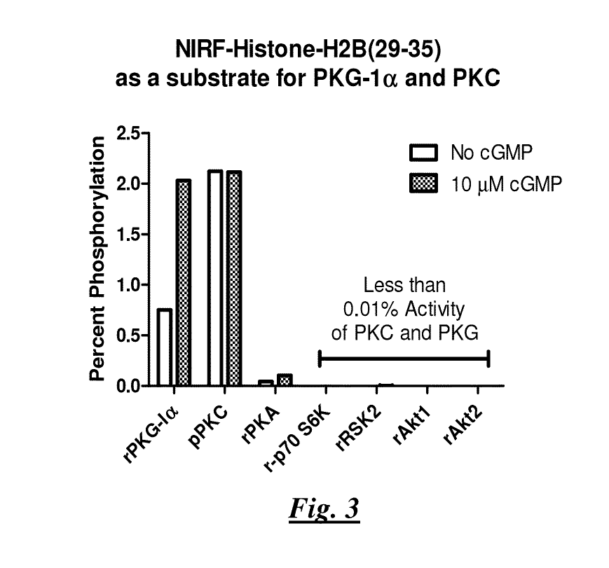

One embodiment, as set forth in FIG. 1, shows a schematic of the novel NIRF-Histone-H2B(29-35) peptide that can be used as a substrate for simultaneously measuring PKC and PKG kinase activities in biological/clinical samples. The specificity of NIRF-Histone-H2B(29-35) for PKC and PKG kinases is unique to some embodiments of the disclosure and has been experimentally determined to be selective for certain isoforms of PKC (FIG. 3). Specifically, the NIRF-Histone-H2B(29-35) is selective for 7 of the 12 known PKC isoforms listed in FIG. 2. The values in FIG. 2 were measured using a γ-33P ATP radioactive kinase assay.

To detect changes in phosphorylation of the peptide in a radioactive-free assay, a near infrared fluorescent dye, AlexaFluor750 (Life Technologies™ Corporation), can be chemically conjugated to the synthesized peptide to make a complete NIRF-Histone-H2B(29-35). The near-infrared fluorescent dye is not limited to AlexaFluor750, but rather can be another near-infrared fluorescent dyes that labels the NIRF-Histone-H2B(29-35). Examples of these dyes and the excitation/emission values are listed in Table 2. Due to Life Technologies™ Corporation's trade secret protection of AlexaFluor750, the actual ionic charge of the compound is unknown, but is in the negative range. Therefore, the net charge of the nonphosphorylated species of NIRF-Histone-H2B(29-35) at pH=7.0 is ≦+3 or ≦+1 for the phosphorylated species. The completed reaction is electrophoretically separated in a buffer of pH=10.0 and in these conditions the net charge of the nonphosphorylated species of NIRF-Histone-H2B(29-35) is ≦+1 or ≦−1 for the phosphorylated species.

The full design of NIRF-Histone-H2B(29-35) shown in FIG. 1 includes:
a. AlexaFluor750 covalently bound to a cysteine (C) side chain.
b. C covalently bound to 6-aminohexanoic acid (Ahx) used to distance the core seven amino acids from the AlexaFluor750 dye and limits interference from the dye in biological reactions.
c. Ahx is covalently bound to the core amino acid sequence: RKRSRKE terminating with an amino (NH2) cap.
d. R=Arginine, K=Lysine, S=Serine, E=Glutamic Acid.

Table 3 is the list of reagents used in the radioactive-free kinase reaction mix used in the disclosure. Each reagent has been experimentally tested to be the optimum concentration used for a complete radioactive-free kinase reaction using NIRF-Histone-H2B(29-35). All reagents listed in Table 3 are commercially available.

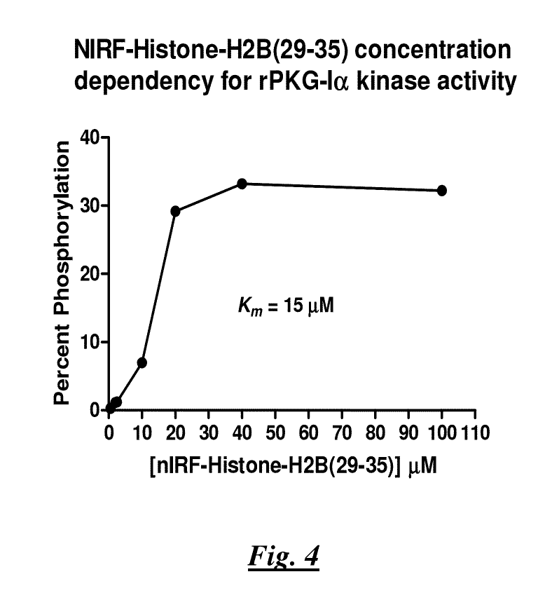

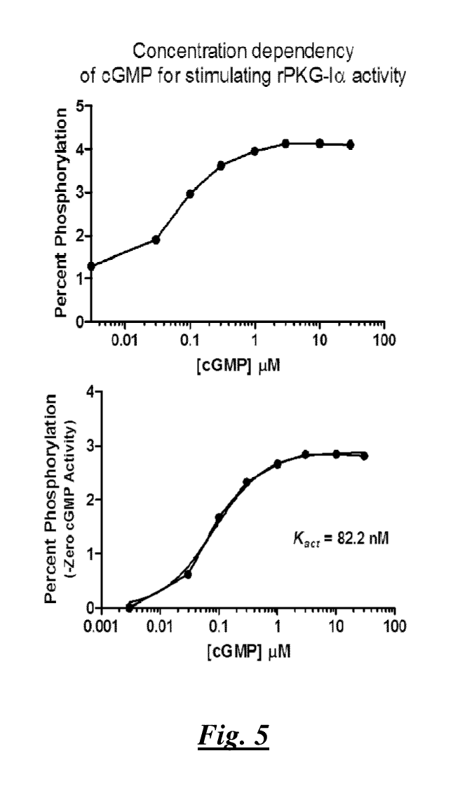

The concentration of NIRF-Histone-H2B(29-35) used in the radioactive-free kinase mix of some embodiments of the disclosure provides 20 μM. This concentration is close to the Km (which is =15 μM) that has been experimentally determined using recombinant PKG-Iα kinase (FIG. 4). Additionally, PKG-Iα can be fully activated by cyclic guanosine monophosphate (cGMP) at a concentration of 10 μM under our assay conditions and can be the concentration that is used in all experiments in which cGMP is used (FIG. 5).

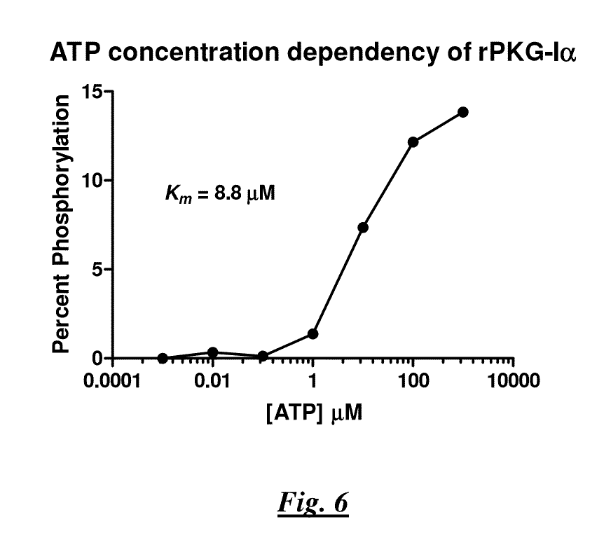

A unique advantage of some embodiments of the disclosure, when compared to previous techniques, is the use of a biologically-relevant concentration of 1 mM ATP. The physiological levels of ATP within mammalian cells has been estimated to be 1-10 mM. PKG or certain PKC isoforms utilize ATP as a phosphate donor molecule to transfer the −2 charged γ-phosphate group to the serine residue (‘S’ in FIG. 1) of the NIRF-Histone-H2B(29-35). FIG. 6 demonstrates that PKG-Iα, the most common isoform of PKG, has a Km for ATP that is 8.8 μM but requires 1 mM in order to reach full activity when analysis under the present experimental conditions.

A set of reagents for the radioactive-free kinase reaction mix can include: a buffer solution of 20 mM TRIS-HCl at pH 7.4, 1 mM ATP (FIG. 6), an ATP cofactor salt magnesium chloride (10 mM MgCl₂), a reducing agent dithiothreitol (10 mM DTT) to prevent artificial oxidation of the protein kinases during sample preparation and kinase reaction, 20 μM of the peptide NIRF-Histone-H2B(29-35) (FIG. 4), and the radioactive-free kinase reaction mixture can include versions with or without the PKG allosteric activator, cGMP at 10 μM (FIG. 5).

In other embodiments, the disclosure includes two buffers that can be selected to prepare a sample to be tested; the homogenizing buffer (Table 4) or purified/recombinant protein buffer recommended by the manufacturer. Although the volume of biological sample can be adjusted according to preference, 10 μL per kinase reaction is typically used, which includes the radioactive-free kinase reaction mixture and cell lysate/homogenized tissue sample or the purified/recombinant protein buffer. The disclosure protocol using the radioactive-free kinase reaction mixture, listed in Table 4, is outlined in Table 5 and illustrated in FIG. 7.

Table 4 is a list of reagents that can be included in the homogenizing buffer. Each reagent has been previously experimentally tested to be the optimum concentration used for a complete radioactive-free kinase reaction to measure PKG kinase activity. The reagents for homogenizing buffer include: 20 mM Potassium phosphate (pH 7.0), 10 mM EDTA, 0.5 mM isobutylmethylxanthine, and 10 mM DTT. The purpose of each reagent is listed in Table 4.

The incubation time of some embodiments of the present disclosure may be only 2 minutes in length and therefore these components may not be needed for addition to the radioactive-free kinase reaction mixture. Others using new methodology can chose to use protease and phosphatase inhibitors, but some protease and phosphatase inhibitor can interfere with and alter the measurement of catalytic activity of certain protein kinases.

Many, if not all, of the components in the radioactive-free kinase reaction mixture listed in Table 3 and homogenizing buffer listed in Table 4 are able to be modified to suit the particular needs of the researcher. Examples of modifications to the radioactive-free kinase mixture and/or homogenizing buffer are listed below:

The concentration of ATP and Magnesium chloride can be adjusted above and below the recommended amounts.

Any reducing agent other than DTT can be used in the radioactive—free kinase reaction mixture and homogenizing buffer including, but not limited to: beta-mercaptoethanol (beta-MCE), reduced glutathione, reduced cysteine, reduced ascorbic acid, reduced vitamin E, NADH, and Tris(2-carboxyethyl)phosphine (TCEP).

The disclosure allows for addition of phosphatase inhibitors and/or protease inhibitors to the above radioactive-free kinase mixture in singleton or in a combination (cocktail). However, due to the potential interference of these compounds, these additional compounds are not necessary to include in the radioactive-free kinase reaction mixture if the reaction time is kept short. In one aspect of the disclosure, the radioactive-free kinase reaction mixture is able to support the addition of the following phosphatase inhibitors as an example, but not limited to: sodium fluoride, sodium orthovanadate, β-glycerophosphate, sodium pyrophosphate, microcystin, and okadaic acid. Likewise, the radioactive-free kinase reaction mixture is able to support the addition of the following protease inhibitors as an example, but not limited to: AEBSF-HCl, aprotinin, bestatin, E-64, leupeptin, pepstatin A, benzamidine, and PMSF.

Detergents (aiding in cell lysis) may be utilized in the radioactive-free kinase reaction mixture. These detergents include, but are not limited to: Triton X-100, Triton X-114, Tween 20, Tween 80, NP-40, CHAPS, CHAPSO, Brij-35, Brij-58, octyl glucoside, octyl thioglucoside, digitonin, and SDS. However, at higher concentrations these detergents may interfere with the kinase reaction and thus need to be tested.

Carrier agents can be utilized for reducing the loss of specific proteins (e.g. kinases), peptides (e.g. substrate), and other chemicals for the lysis/homogenizing buffer and kinase reaction mixture because of sticking to the surface of pipette tips, tubing, reaction and preparatory containers, etc. These carrier agents include, but are not limited to: bovine serum albumin (BSA), siliconization, gamma globulin, casein, and/or Prionex (Centerchem).

The buffer system can also be altered to suit the specific needs of the researcher. Changes to the buffer system include, but are not limited to: substituting phosphate buffer for Tris-HCl, substituting MOPS for Tris-HCl or substituting HEPES for Tris-HCl.

Table 5 is the disclosure protocol that is illustrated as a work flow in FIG. 7. Biological samples can be prepared in the homogenizing buffer (Table 4) using a variety of techniques. These techniques include, but are not limited to: sonication, dounce-homogenation, the use of a tissue grinder, and/or a detergent specialized for lysing cells. Once a biological sample is prepared in the specially formulated homogenizing buffer from Table 4 or purified/recombinant protein buffer recommended by the manufacturer (2) included in the disclosure, the sample can be added to a sterile microfuge tube containing the radioactive-free kinase reaction mixture containing NIRF-Histone-H2B(29-35) (Table 3 and FIG. 1) (4). The kinase reaction typically requires an incubation chamber capable of reaching a temperature of 30° C. to incubate the samples. The time and temperature of incubation is determined by the researcher, but short incubation times of 2 minutes can be used.

In various embodiments, the linearity of the kinase activity measured in biological samples is diminished severely after 2 minutes at 30° C. and use of a short incubation time (kinase reaction time) of 1 or 2 minutes can be utilized. Other temperatures, such as 0° C., may permit longer incubation times that maintain the linearity of the reaction due to a reduction in the dissociation of activator molecules (e.g. cGMP) as well as to the slower proteolysis and phosphatase activity that may cause decreased measurement of protein kinase activity.

During the reaction, a biological sample containing active isoforms of PKC, PKG or both kinases can convert a percentage of the native species of NIRF-Histone-H2B(29-35) into a phosphorylated NIRF-Histone-H2B(29-35) (species 2 (P)). The reaction can be terminated using 150 mM Ethylenediaminetetraacetic acid (EDTA).

After the incubation procedure has been completed, agarose gel loading dye can be added to the terminated reaction and the contents (4) are loaded into the wells (8) of a 1% agarose gel (6) using transfer pipettes. The 1% agarose gel, made from Tris/Borate/EDTA buffer pH=10.0, can be situated in an electrophoretic box filled with electrophoresis buffer pH=10.0. A negative terminal (10) and positive terminal (12) can connect the electrophoretic box to a power source (14). An electrophoretic field can be generated using a power source running at 75-150 V for 90 minutes (14). The phosphorylation of NIRF-Histone-H2B(29-35) by PKC and/or PKG kinases typically changes the net charge of NIRF-Histone-H2B(29-35) from more positive to more negative. In the pH=10.0 environment of the electrophoresis buffer the difference in charge is: nonphosphorylated=+1 and phosphorylated=−1. During the electrophoretic separation step, this difference in charge allows for the migration of NIRF-Histone-H2B(29-35) (Species 1) (16) to the negatively charged cathode and NIRF-Histone-H2B(29-35) (Species 2 (P)) towards the positively charged anode (18). After electrophoretic separation, the gel is scanned using a near infrared fluorescence (NIRF) imager (20) with lasers capable of generating light at 700-800 nm wavelengths (e.g. LI-COR Odyssey®). The scanned image can be quantified using imaging software available for the near infrared fluorescence scanner (e.g. LI-COR Odyssey® Application Software Version 2.1).

Some embodiments of the disclosure can be adjusted to the specific needs of the researcher in a variety of ways. However, to discriminate between the individual catalytic activities of PKC and PKG, control reaction samples can be compared to reaction samples that include a selective inhibitor or inhibitors of kinase activity. In various embodiments, this disclosure makes use of four isoform-selective PKC inhibitors (AEB071, LY333-531, Gö 6976, and Gö 6983) that cover the range of PKC isoforms in-which NIRF-Histone-H2B(29-35) is a known substrate (see the data shown in FIG. 2). For complete inhibition of the PKC isoforms in our disclosure, the four inhibitors can be added to the reaction mixture in equal amounts of 3-10 μM. FIG. 8 is the molecular structures of the four specific PKC inhibitors. These inhibitors can be added to cell media, the homogenizing buffer/lysis buffer or purified/recombinant protein buffer, the radioactive-free kinase reaction mixture, or a combination of all components listed.

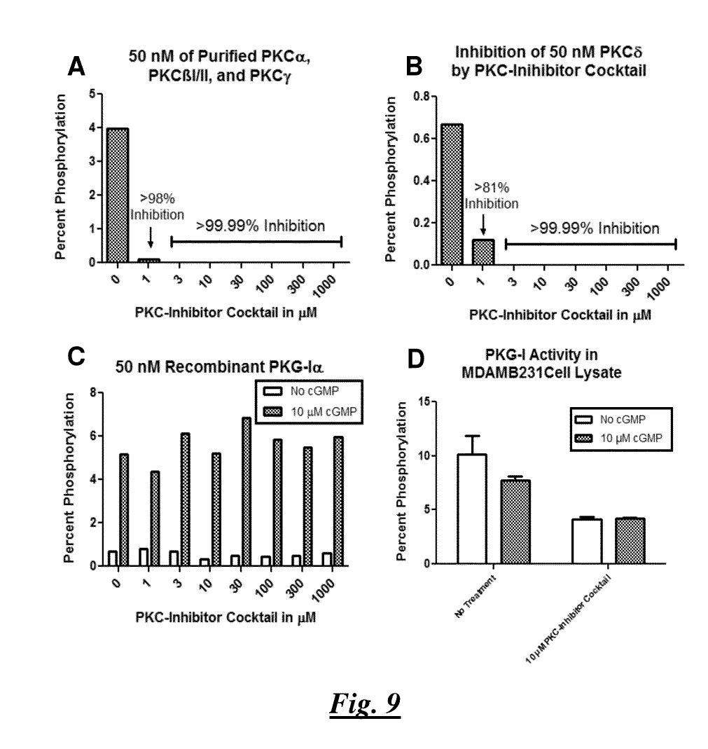

As proof of principle, a cocktail of the inhibitors from FIG. 8 selective for the PKC isoforms in FIG. 2 were added to the radioactive-free kinase mixture from Table 3 and tested for their ability to inhibit PKC kinase activity (illustrated in FIG. 9). Complete inhibition of PKC kinase activity was achieved when 3 μM of each PKC inhibitor from FIG. 8 was added as a cocktail to the reaction (FIG. 9). However, none of the concentrations of the PKC inhibitor cocktail were able to have a measurable effect on PKG-Iα kinase activity (FIG. 9C), making this PKC-inhibitor cocktail a useful tool in discerning the activity of PKC and PKG in biological/clinical samples. Cell lysates prepared from the breast cancer cell line MDA-MB-231 were used to test this hypothesis. Samples of MDA-MB-231 cell lysates were compared with and without the addition of 10 μM of the PKC-inhibitor cocktail and it was determined that PKG contributed ˜52% of the kinase activity measured using NIRF-histone-H2B(29-35) (FIG. 9D).

In various embodiments, this disclosure describes all protein kinase inhibitors of all protein kinases to be used in singleton or in combination in experiments and screening procedures based on this novel NIRF-based protein kinase activity methodology. Table 6 lists possible kinase inhibitors that can be used as a component in this disclosure.

In other embodiments, this disclosure provides a substrate for phosphorylation by a protein kinase comprising a core peptide having the Formula (I): (N-terminus)-Arginine-Lysine-Arginine-Serine-Arginine-Lysine-Glutamic-acid-(C-terminus) and

an indicator component covalently bonded to the core peptide.

In one embodiment, the substrate further includes a linker covalently bonded to and disposed between each of the core peptide and the indicator component. In another embodiment, the linker is directly covalently bonded to the C-terminus of the core peptide and to the indicator component. In a further embodiment, the linker is indirectly covalently bonded to the C-terminus of the core peptide and to the indicator component. Alternatively, the linker may be directly covalently bonded to the C-terminus of the core peptide and indirectly covalently bonded to the indicator component. Even further, the linker may be indirectly covalently bonded to the C-terminus of the core peptide and directly covalently bonded to the indicator component. Still further, the linker may include a linear aminoalkanoic acid having at least six carbon atoms for spacing the indicator component from the core peptide. In another embodiment, the linear aminoalkanoic acid is 6-aminohexanoic acid. Even further, the indicator component may include a fluorophore. Further, the indicator component may be excited by light at a wavelength of from about 700 nm to about 1000 nm and subsequently emit light at a wavelength of from about 700 nm to about 1000 nm. Alternatively, the indicator component may be excited by light at a wavelength of from about 10 nm to about 380 nm and subsequently emit light at a wavelength of from about 10 nm to about 380 nm. In other embodiments, the substrate includes an amino acid anchor covalently bonded to and disposed between each of the linker and the indicator component. The amino acid anchor may be further defined as cysteine. Moreover, the protein kinase may be sufficiently capable of phosphorylating the core peptide to form a phosphorylated substrate. Even further, the protein kinase may be a protein kinase C, a protein kinase G, or a combination thereof. Moreover, in one embodiment, only the protein kinase C and protein kinase G are sufficiently capable of phosphorylating the core peptide to form the phosphorylated substrate.

This disclosure also provides a method for detecting a phosphorylated substrate. The method includes the steps of providing a non-phosphorylated substrate comprising a core peptide, phosphorylating the core peptide with a protein kinase to form the phosphorylated substrate; and detecting the phosphorylated substrate. In the method, the non-phosphorylated substrate includes; a core peptide having the Formula (I): (N-terminus)-Arginine-Lysine-Arginine-Serine-Arginine-Lysine-Glutamic-acid-(C-terminus) and an indicator component covalently bonded to the core peptide. In one embodiment, the method further includes the steps of combining the non-phosphorylated substrate and the protein kinase to form a reaction mixture; incubating the reaction mixture for, in some embodiments, no greater than twenty minutes at about 30° C.; combining the reaction mixture and EDTA to terminate the phosphorylation of the non-phosphorylated substrate by the protein kinase; and separating the phosphorylated substrate from the non-phosphorylated substrate. In further embodiments, the step of separating is further defined as separating the phosphorylated substrate from the non-phosphorylated substrate by electrophoresis wherein the phosphorylated substrate has a negative charge, and the non-phosphorylated substrate has a positive charge, each in the presence of a buffer composition.

Alternatively, the step of detecting the phosphorylated substrate can include the steps of: exciting the indicator component of the phosphorylated substrate with light at a wavelength of from about 700 nm to about 1000 nm such that the indicator component emits light at a wavelength of from about 700 nm to about 1000 nm; and quantifying the light emitted.

This disclosure further provides a substrate for phosphorylation by a protein kinase comprising: a core peptide having the Formula (I): (N-terminus)-Arginine-Lysine-Arginine-Serine-Arginine-Lysine-Glutamic-acid-(C-terminus); a linker comprising 6-aminohexanoic acid and directly covalently bonded to the C-terminus of the core peptide; an amino acid anchor comprising cysteine and directly covalently bonded to the linker; and an indicator component comprising a fluorophore and directly covalently bonded to the amino acid anchor, wherein the indicator component is excited by light at a wavelength of from about 700 nm to about 1000 nm and subsequently emits light at a wavelength of from about 700 nm to about 1000 nm, wherein the protein kinase is protein kinase C, protein kinase G, or a combination thereof; and wherein only the protein kinase C and protein kinase G are sufficiently capable of phosphorylating the core peptide to form the phosphorylated substrate.

While exemplary systems and methods, and applications of methods of the disclosure, have been described herein, it should also be understood that the foregoing is only illustrative of a few particular embodiments with exemplary and/or preferred features, as well as principles of the disclosure, and that various modifications can be made by those skilled in the art without departing from the scope and spirit of the disclosure. Therefore, the described embodiments should not be considered as limiting of the scope of the disclosure in any way. Accordingly, the disclosure embraces alternatives, modifications and variations which fall within the spirit and scope of the disclosure and claims as set forth herein, including any equivalents thereto.

TABLE 1
LIST OF KNOWN PROTEIN KINASE ENZYMES IN HUMAN AND MOUSE

| | | | | |
|---|---|---|---|---|
| Common | Uniprot Name | Uniprot | Uniprot Name | Uniprot |
| Name | (Human) | ID | (Mouse) | ID |

AGC Ser/Thr protein kinase family

| | | | | |
|---|---|---|---|---|
| ADRBK1 | ARBK1_HUMAN | (P25098) | ARBK1_MOUSE | (Q99MK8) |
| ADRBK2 | ARBK2_HUMAN | (P35626) | ARBK2_MOUSE | (Q3UYH7) |
| AKT1 | AKT1_HUMAN | (P31749) | AKT1_MOUSE | (P31750) |
| AKT2 | AKT2_HUMAN | (P31751) | AKT2_MOUSE | (Q60823) |
| AKT3 | AKT3_HUMAN | (Q9Y243) | AKT3_MOUSE | (Q9WUA6) |
| CDC42BPA | MRCKA_HUMAN | (Q5VT25) | MRCKA_MOUSE | (Q3UU96) |
| CDC42BPB | MRCKB_HUMAN | (Q9Y5S2) | MRCKB_MOUSE | (Q7TT50) |
| CDC42BPG | MRCKG_HUMAN | (Q6DT37) | MRCKG_MOUSE | (Q80UW5) |
| CIT | CTRO_HUMAN | (O14578) | CTRO_MOUSE | (P49025) |
| DMPK | DMPK_HUMAN | (Q09013) | DMPK_MOUSE | (P54265) |
| GRK1 | RK_HUMAN | (Q15835) | RK_MOUSE | (Q9WVL4) |
| GRK4 | GRK4_HUMAN | (P32298) | GRK4_MOUSE | (O70291) |
| GRK5 | GRK5_HUMAN | (P34947) | GRK5_MOUSE | (Q8VEB1) |
| GRK6 | GRK6_HUMAN | (P43250) | GRK6_MOUSE | (O70293) |
| GRK7 | GRK7_HUMAN | (Q8WTQ7) | | |
| LATS1 | LATS1_HUMAN | (O95835) | LATS1_MOUSE | (Q8BYR2) |
| LATS2 | LATS2_HUMAN | (Q9NRM7) | LATS2_MOUSE | (Q7TSJ6) |
| MAST1 | MAST1_HUMAN | (Q9Y2H9) | MAST1_MOUSE | (Q9R1L5) |
| MAST2 | MAST2_HUMAN | (Q6P0Q8) | MAST2_MOUSE | (Q60592) |
| MAST3 | MAST3_HUMAN | (O60307) | MAST3_MOUSE | (Q3U214) |
| MAST4 | MAST4_HUMAN | (O15021) | MAST4_MOUSE | (Q811L6) |
| MASTL | GWL_HUMAN | (Q96GX5) | GWL_MOUSE | (Q8C0P0) |
| PDPK1 | PDPK1_HUMAN | (O15530) | PDPK1_MOUSE | (Q9Z2A0) |
| PDPK2 | PDPK2_HUMAN | (Q6A1A2) | | |
| PKN1 | PKN1_HUMAN | (Q16512) | PKN1_MOUSE | (P70268) |
| PKN2 | PKN2_HUMAN | (Q16513) | PKN2_MOUSE | (Q8BWW9) |
| PKN3 | PKN3_HUMAN | (Q6P5Z2) | PKN3_MOUSE | (Q8K045) |
| PRKACA | KAPCA_HUMAN | (P17612) | KAPCA_MOUSE | (P05132) |
| PRKACB | KAPCB_HUMAN | (P22694) | KAPCB_MOUSE | (P68181) |
| PRKACG | KAPCG_HUMAN | (P22612) | | |
| PRKCA | KPCA_HUMAN | (P17252) | KPCA_MOUSE | (P20444) |
| PRKCB | KPCB_HUMAN | (P05771) | KPCB_MOUSE | (P68404) |
| PRKCD | KPCD_HUMAN | (Q05655) | KPCD_MOUSE | (P28867) |
| PRKCE | KPCE_HUMAN | (Q02156) | KPCE_MOUSE | (P16054) |
| PRKCG | KPCG_HUMAN | (P05129) | KPCG_MOUSE | (P63318) |
| PRKCH | KPCL_HUMAN | (P24723) | KPCL_MOUSE | (P23298) |
| PRKCI | KPCI_HUMAN | (P41743) | KPCI_MOUSE | (Q62074) |
| PRKCQ | KPCT_HUMAN | (Q04759) | KPCT_MOUSE | (Q02111) |
| PRKCZ | KPCZ_HUMAN | (Q05513) | KPCZ_MOUSE | (Q02956) |
| PRKG1 | KGP1_HUMAN | (Q13976) | KGP1_MOUSE | (P0C605) |
| PRKG2 | KGP2_HUMAN | (Q13237) | KGP2_MOUSE | (Q61410) |
| PRKX | PRKX_HUMAN | (P51817) | PRKX_MOUSE | (Q922R0) |
| ROCK1 | ROCK1_HUMAN | (Q13464) | ROCK1_MOUSE | (P70335) |
| ROCK2 | ROCK2_HUMAN | (O75116) | ROCK2_MOUSE | (P70336) |
| RPS6KA1 | KS6A1_HUMAN | (Q15418) | KS6A1_MOUSE | (P18653) |
| RPS6KA2 | KS6A2_HUMAN | (Q15349) | KS6A2_MOUSE | (Q9WUT3) |
| RPS6KA3 | KS6A3_HUMAN | (P51812) | KS6A3_MOUSE | (P18654) |
| RPS6KA4 | KS6A4_HUMAN | (O75676) | KS6A4_MOUSE | (Q9Z2B9) |
| RPS6KA5 | KS6A5_HUMAN | (O75582) | KS6A5_MOUSE | (Q8C050) |
| RPS6KA6 | KS6A6_HUMAN | (Q9UK32) | KS6A6_MOUSE | (Q7TPS0) |
| RPS6KB1 | KS6B1_HUMAN | (P23443) | KS6B1_MOUSE | (Q8BSK8) |
| RPS6KB2 | KS6B2_HUMAN | (Q9UBS0) | KS6B2_MOUSE | (Q9Z1M4) |
| SGK1 | SGK1_HUMAN | (O00141) | SGK1_MOUSE | (Q9WVC6) |
| SGK2 | SGK2_HUMAN | (Q9HBY8) | SGK2_MOUSE | (Q9QZS5) |
| SGK3 | SGK3_HUMAN | (Q96BR1) | SGK3_MOUSE | (Q9ERE3) |
| STK38 | STK38_HUMAN | (Q15208) | STK38_MOUSE | (Q91VJ4) |
| STK38L | ST38L_HUMAN | (Q9Y2H1) | ST38L_MOUSE | (Q7TSE6) |

CAMK Ser/Thr protein kinase family

| | | | | |
|---|---|---|---|---|
| | | | SMKX_MOUSE | (Q8C0X8) |
| BRSK1 | BRSK1_HUMAN | (Q8TDC3) | BRSK1_MOUSE | (Q5RJI5) |
| BRSK2 | BRSK2_HUMAN | (Q8IWQ3) | BRSK2_MOUSE | (Q69Z98) |
| CAMK1 | KCC1A_HUMAN | (Q14012) | KCC1A_MOUSE | (Q91YS8) |
| CAMK1D | KCC1D_HUMAN | (Q8IU85) | KCC1D_MOUSE | (Q8BW96) |
| CAMK1G | KCC1G_HUMAN | (Q96NX5) | KCC1G_MOUSE | (Q91VB2) |
| CAMK2A | KCC2A_HUMAN | (Q9UQM7) | KCC2A_MOUSE | (P11798) |
| CAMK2B | KCC2B_HUMAN | (Q13554) | KCC2B_MOUSE | (P28652) |
| CAMK2D | KCC2D_HUMAN | (Q13557) | KCC2D_MOUSE | (Q6PHZ2) |
| CAMK2G | KCC2G_HUMAN | (Q13555) | KCC2G_MOUSE | (Q923T9) |
| CAMK4 | KCC4_HUMAN | (Q16566) | KCC4_MOUSE | (P08414) |
| CAMKV | CAMKV_HUMAN | (Q8NCB2) | CAMKV_MOUSE | (Q3UHL1) |
| CASK | CSKP_HUMAN | (O14936) | CSKP_MOUSE | (O70589) |
| CHEK1 | CHK1_HUMAN | (O14757) | CHK1_MOUSE | (O35280) |
| CHEK2 | CHK2_HUMAN | (O96017) | CHK2_MOUSE | (Q9Z265) |
| DAPK1 | DAPK1_HUMAN | (P53355) | DAPK1_MOUSE | (Q80YE7) |
| DAPK2 | DAPK2_HUMAN | (Q9UIK4) | DAPK2_MOUSE | (Q8VDF3) |
| DAPK3 | DAPK3_HUMAN | (O43293) | DAPK3_MOUSE | (O54784) |
| DCLK1 | DCLK1_HUMAN | (O15075) | DCLK1_MOUSE | (Q9JLM8) |
| DCLK2 | DCLK2_HUMAN | (Q8N568) | DCLK2_MOUSE | (Q6PGN3) |
| DCLK3 | DCLK3_HUMAN | (Q9C098) | DCLK3_MOUSE | (Q8BWQ5) |
| Gm4776 | | | SMKW_MOUSE | (Q8C0V7) |
| Gm4922 | | | SMKZ_MOUSE | (Q8C0N0) |
| Gm7168 | | | SMKY_MOUSE | (A0AUV4) |
| HUNK | HUNK_HUMAN | (P57058) | HUNK_MOUSE | (O88866) |
| KALRN | KALRN_HUMAN | (O60229) | KALRN_MOUSE | (A2CG49) |
| MAPKAPK2 | MAPK2_HUMAN | (P49137) | MAPK2_MOUSE | (P49138) |
| MAPKAPK3 | MAPK3_HUMAN | (Q16644) | MAPK3_MOUSE | (Q3UMW7) |
| MAPKAPK5 | MAPK5_HUMAN | (Q8IW41) | MAPK5_MOUSE | (O54992) |
| MARK1 | MARK1_HUMAN | (Q9P0L2) | MARK1_MOUSE | (Q8VHJ5) |
| MARK2 | MARK2_HUMAN | (Q7KZI7) | MARK2_MOUSE | (Q05512) |
| MARK3 | MARK3_HUMAN | (P27448) | MARK3_MOUSE | (Q03141) |
| MARK4 | MARK4_HUMAN | (Q96L34) | MARK4_MOUSE | (Q8CIP4) |
| MELK | MELK_HUMAN | (Q14680) | MELK_MOUSE | (Q61846) |
| MKNK1 | MKNK1_HUMAN | (Q9BUB5) | MKNK1_MOUSE | (O08605) |
| MKNK2 | MKNK2_HUMAN | (Q9HBH9) | MKNK2_MOUSE | (Q8CDB0) |
| MYLK | MYLK_HUMAN | (Q15746) | MYLK_MOUSE | (Q6PDN3) |
| MYLK2 | MYLK2_HUMAN | (Q9H1R3) | MYLK2_MOUSE | (Q8VCR8) |
| MYLK3 | MYLK3_HUMAN | (Q32MK0) | MYLK3_MOUSE | (Q3UIZ8) |
| MYLK4 | MYLK4_HUMAN | (Q86YV6) | MYLK4_MOUSE | (Q5SUV5) |
| NIM1 | NIM1_HUMAN | (Q8IY84) | NIM1_MOUSE | (Q8BHI9) |
| NUAK1 | NUAK1_HUMAN | (O60285) | NUAK1_MOUSE | (Q641K5) |
| NUAK2 | NUAK2_HUMAN | (Q9H093) | NUAK2_MOUSE | (Q8BZN4) |
| OBSCN | OBSCN_HUMAN | (Q5VST9) | OBSCN_MOUSE | (A2AAJ9) |
| PASK | PASK_HUMAN | (Q96RG2) | PASK_MOUSE | (Q8CEE6) |
| PHKG1 | PHKG1_HUMAN | (Q16816) | PHKG1_MOUSE | (P07934) |
| PHKG2 | PHKG2_HUMAN | (P15735) | PHKG2_MOUSE | (Q9DB30) |
| PIM1 | PIM1_HUMAN | (P11309) | PIM1_MOUSE | (P06803) |
| PIM2 | PIM2_HUMAN | (Q9P1W9) | PIM2_MOUSE | (Q62070) |
| PIM3 | PIM3_HUMAN | (Q86V86) | PIM3_MOUSE | (P58750) |
| PNCK | KCC1B_HUMAN | (Q6P2M8) | KCC1B_MOUSE | (Q9QYK9) |
| PRKAA1 | AAPK1_HUMAN | (Q13131) | AAPK1_MOUSE | (Q5EG47) |
| PRKAA2 | AAPK2_HUMAN | (P54646) | AAPK2_MOUSE | (Q8BRK8) |
| PRKD1 | KPCD1_HUMAN | (Q15139) | KPCD1_MOUSE | (Q62101) |
| PRKD2 | KPCD2_HUMAN | (Q9BZL6) | KPCD2_MOUSE | (Q8BZ03) |
| PRKD3 | KPCD3_HUMAN | (O94806) | KPCD3_MOUSE | (Q8K1Y2) |
| PSKH1 | KPSH1_HUMAN | (P11801) | KPSH1_MOUSE | (Q91YA2) |
| PSKH2 | KPSH2_HUMAN | (Q96QS6) | | |
| SIK1 | SIK1_HUMAN | (P57059) | SIK1_MOUSE | (Q60670) |
| SIK2 | SIK2_HUMAN | (Q9H0K1) | SIK2_MOUSE | (Q8CFH6) |
| SIK3 | SIK3_HUMAN | (Q9Y2K2) | SIK3_MOUSE | (Q6P4S6) |
| SNRK | SNRK_HUMAN | (Q9NRH2) | SNRK_MOUSE | (Q8VDU5) |
| SPEG | SPEG_HUMAN | (Q15772) | SPEG_MOUSE | (Q62407) |
| STK11 | STK11_HUMAN | (Q15831) | STK11_MOUSE | (Q9WTK7) |
| STK17A | ST17A_HUMAN | (Q9UEE5) | | |
| STK17B | ST17B_HUMAN | (O94768) | ST17B_MOUSE | (Q8BG48) |
| STK33 | STK33_HUMAN | (Q9BYT3) | STK33_MOUSE | (Q924X7) |
| STK40 | STK40_HUMAN | (Q8N2I9) | STK40_MOUSE | (Q7TNL3) |
| Smok2a | | | SMK2A_MOUSE | (Q9QYZ6) |
| Smok2b | | | SMK2B_MOUSE | (Q9QYZ3) |
| Smok3a | | | SMK3_MOUSE | (Q9QYZ5) |
| TRIB1 | TRIB1_HUMAN | (Q96RU8) | TRIB1_MOUSE | (Q8K4K4) |
| TRIB2 | TRIB2_HUMAN | (Q92519) | TRIB2_MOUSE | (Q8K4K3) |
| TRIB3 | TRIB3_HUMAN | (Q96RU7) | TRIB3_MOUSE | (Q8K4K2) |
| TRIO | TRIO_HUMAN | (O75962) | TRIO_MOUSE | (Q0KL02) |
| TSSK1B | TSSK1_HUMAN | (Q9BXA7) | TSSK1_MOUSE | (Q61241) |
| TSSK2 | TSSK2_HUMAN | (Q96PF2) | TSSK2_MOUSE | (O54863) |
| TSSK3 | TSSK3_HUMAN | (Q96PN8) | TSSK3_MOUSE | (Q9D2E1) |
| TSSK4 | TSSK4_HUMAN | (Q6SA08) | TSSK4_MOUSE | (Q9D411) |
| TSSK6 | TSSK6_HUMAN | (Q9BXA6) | TSSK6_MOUSE | (Q925K9) |
| TTN | TITIN_HUMAN | (Q8WZ42) | TITIN_MOUSE | (A2ASS6) |
| Tssk5 | | | TSSK5_MOUSE | (Q8C1R0) |

CK1 Ser/Thr protein kinase family

| | | | | |
|---|---|---|---|---|
| CSNK1A1 | KC1A_HUMAN | (P48729) | KC1A_MOUSE | (Q8BK63) |
| CSNK1A1L | KC1AL_HUMAN | (Q8N752) | | |
| CSNK1D | KC1D_HUMAN | (P48730) | KC1D_MOUSE | (Q9DC28) |
| CSNK1E | KC1E_HUMAN | (P49674) | KC1E_MOUSE | (Q9JMK2) |
| CSNK1G1 | KC1G1_HUMAN | (Q9HCP0) | KC1G1_MOUSE | (Q8BTH8) |
| CSNK1G2 | KC1G2_HUMAN | (P78368) | KC1G2_MOUSE | (Q8BVP5) |
| CSNK1G3 | KC1G3_HUMAN | (Q9Y6M4) | KC1G3_MOUSE | (Q8C4X2) |
| TTBK1 | TTBK1_HUMAN | (Q5TCY1) | TTBK1_MOUSE | (Q6PCN3) |
| TTBK2 | TTBK2_HUMAN | (Q6IQ55) | TTBK2_MOUSE | (Q3UVR3) |
| VRK1 | VRK1_HUMAN | (Q99986) | VRK1_MOUSE | (Q80X41) |
| VRK2 | VRK2_HUMAN | (Q86Y07) | VRK2_MOUSE | (Q8BN21) |
| VRK3 | VRK3_HUMAN | (Q8IV63) | VRK3_MOUSE | (Q8K3G5) |

CMGC Ser/Thr protein kinase family

| | | | | |
|---|---|---|---|---|
| CDK1 | CDK1_HUMAN | (P06493) | CDK1_MOUSE | (P11440) |
| CDK10 | CDK10_HUMAN | (Q15131) | CDK10_MOUSE | (Q3UMM4) |
| CDK11A | CD11A_HUMAN | (Q9UQ88) | | |
| CDK11B | CD11B_HUMAN | (P21127) | CD11B_MOUSE | (P24788) |
| CDK12 | CDK12_HUMAN | (Q9NYV4) | CDK12_MOUSE | (Q14AX6) |
| CDK13 | CDK13_HUMAN | (Q14004) | CDK13_MOUSE | (Q69ZA1) |
| CDK14 | CDK14_HUMAN | (O94921) | CDK14_MOUSE | (O35495) |
| CDK15 | CDK15_HUMAN | (Q96Q40) | CDK15_MOUSE | (Q3V3A1) |
| CDK16 | CDK16_HUMAN | (Q00536) | CDK16_MOUSE | (Q04735) |
| CDK17 | CDK17_HUMAN | (Q00537) | CDK17_MOUSE | (Q8K0D0) |
| CDK18 | CDK18_HUMAN | (Q07002) | CDK18_MOUSE | (Q04899) |
| CDK19 | CDK19_HUMAN | (Q9BWU1) | CDK19_MOUSE | (Q8BWD8) |
| CDK2 | CDK2_HUMAN | (P24941) | CDK2_MOUSE | (P97377) |
| CDK20 | CDK20_HUMAN | (Q8IZL9) | CDK20_MOUSE | (Q9JHU3) |
| CDK3 | CDK3_HUMAN | (Q00526) | CDK3_MOUSE | (Q80YP0) |
| CDK4 | CDK4_HUMAN | (P11802) | CDK4_MOUSE | (P30285) |
| CDK5 | CDK5_HUMAN | (Q00535) | CDK5_MOUSE | (P49615) |
| CDK6 | CDK6_HUMAN | (Q00534) | CDK6_MOUSE | (Q64261) |
| CDK7 | CDK7_HUMAN | (P50613) | CDK7_MOUSE | (Q03147) |
| CDK8 | CDK8_HUMAN | (P49336) | CDK8_MOUSE | (Q8R3L8) |
| CDK9 | CDK9_HUMAN | (P50750) | CDK9_MOUSE | (Q99J95) |
| CDKL1 | CDKL1_HUMAN | (Q00532) | CDKL1_MOUSE | (Q8CEQ0) |
| CDKL2 | CDKL2_HUMAN | (Q92772) | CDKL2_MOUSE | (Q9QUK0) |
| CDKL3 | CDKL3_HUMAN | (Q8IVW4) | CDKL3_MOUSE | (Q8BLF2) |
| CDKL4 | CDKL4_HUMAN | (Q5MAI5) | CDKL4_MOUSE | (Q3TZA2) |
| CDKL5 | CDKL5_HUMAN | (O76039) | CDKL5_MOUSE | (Q3UTQ8) |
| CLK1 | CLK1_HUMAN | (P49759) | CLK1_MOUSE | (P22518) |
| CLK2 | CLK2_HUMAN | (P49760) | CLK2_MOUSE | (O35491) |
| CLK3 | CLK3_HUMAN | (P49761) | CLK3_MOUSE | (O35492) |
| CLK4 | CLK4_HUMAN | (Q9HAZ1) | CLK4_MOUSE | (O35493) |
| DYRK1A | DYR1A_HUMAN | (Q13627) | DYR1A_MOUSE | (Q61214) |
| DYRK1B | DYR1B_HUMAN | (Q9Y463) | DYR1B_MOUSE | (Q9Z188) |
| DYRK2 | DYRK2_HUMAN | (Q92630) | DYRK2_MOUSE | (Q5U4C9) |
| DYRK3 | DYRK3_HUMAN | (O43781) | DYRK3_MOUSE | (Q922Y0) |
| DYRK4 | DYRK4_HUMAN | (Q9NR20) | DYRK4_MOUSE | (Q8BI55) |
| GSK3A | GSK3A_HUMAN | (P49840) | GSK3A_MOUSE | (Q2NL51) |
| GSK3B | GSK3B_HUMAN | (P49841) | GSK3B_MOUSE | (Q9WV60) |
| HIPK1 | HIPK1_HUMAN | (Q86Z02) | HIPK1_MOUSE | (O88904) |
| HIPK2 | HIPK2_HUMAN | (Q9H2X6) | HIPK2_MOUSE | (Q9QZR5) |
| HIPK3 | HIPK3_HUMAN | (Q9H422) | HIPK3_MOUSE | (Q9ERH7) |
| HIPK4 | HIPK4_HUMAN | (Q8NE63) | HIPK4_MOUSE | (Q3V016) |
| ICK | ICK_HUMAN | (Q9UPZ9) | ICK_MOUSE | (Q9JKV2) |
| MAK | MAK_HUMAN | (P20794) | MAK_MOUSE | (Q04859) |
| MAPK1 | MK01_HUMAN | (P28482) | MK01_MOUSE | (P63085) |
| MAPK10 | MK10_HUMAN | (P53779) | MK10_MOUSE | (Q61831) |
| MAPK11 | MK11_HUMAN | (Q15759) | MK11_MOUSE | (Q9WUI1) |
| MAPK12 | MK12_HUMAN | (P53778) | MK12_MOUSE | (O08911) |
| MAPK13 | MK13_HUMAN | (O15264) | MK13_MOUSE | (Q9Z1B7) |
| MAPK14 | MK14_HUMAN | (Q16539) | MK14_MOUSE | (P47811) |
| MAPK15 | MK15_HUMAN | (Q8TD08) | MK15_MOUSE | (Q80Y86) |
| MAPK3 | MK03_HUMAN | (P27361) | MK03_MOUSE | (Q63844) |
| MAPK4 | MK04_HUMAN | (P31152) | MK04_MOUSE | (Q6P5G0) |
| MAPK6 | MK06_HUMAN | (Q16659) | MK06_MOUSE | (Q61532) |
| MAPK7 | MK07_HUMAN | (Q13164) | MK07_MOUSE | (Q9WVS8) |
| MAPK8 | MK08_HUMAN | (P45983) | MK08_MOUSE | (Q91Y86) |
| MAPK9 | MK09_HUMAN | (P45984) | MK09_MOUSE | (Q9WTU6) |
| MOK | MOK_HUMAN | (Q9UQ07) | MOK_MOUSE | (Q9WVS4) |
| NLK | NLK_HUMAN | (Q9UBE8) | NLK_MOUSE | (O54949) |
| PRPF4B | PRP4B_HUMAN | (Q13523) | PRP4B_MOUSE | (Q61136) |
| SRPK1 | SRPK1_HUMAN | (Q96SB4) | SRPK1_MOUSE | (O70551) |
| SRPK2 | SRPK2_HUMAN | (P78362) | SRPK2_MOUSE | (O54781) |
| SRPK3 | SRPK3_HUMAN | (Q9UPE1) | SRPK3_MOUSE | (Q9Z0G2) |

NEK Ser/Thr protein kinase family

| | | | | |
|---|---|---|---|---|
| NEK1 | NEK1_HUMAN | (Q96PY6) | NEK1_MOUSE | (P51954) |
| NEK10 | NEK10_HUMAN | (Q6ZWH5) | NEK10_MOUSE | (Q3UGM2) |
| NEK11 | NEK11_HUMAN | (Q8NG66) | NEK11_MOUSE | (Q8C0Q4) |
| NEK2 | NEK2_HUMAN | (P51955) | NEK2_MOUSE | (O35942) |
| NEK3 | NEK3_HUMAN | (P51956) | NEK3_MOUSE | (Q9R0A5) |
| NEK4 | NEK4_HUMAN | (P51957) | NEK4_MOUSE | (Q9Z1J2) |
| NEK5 | NEK5_HUMAN | (Q6P3R8) | NEK5_MOUSE | (Q7TSC3) |
| NEK6 | NEK6_HUMAN | (Q9HC98) | NEK6_MOUSE | (Q9ES70) |
| NEK7 | NEK7_HUMAN | (Q8TDX7) | NEK7_MOUSE | (Q9ES74) |
| NEK8 | NEK8_HUMAN | (Q86SG6) | NEK8_MOUSE | (Q91ZR4) |
| NEK9 | NEK9_HUMAN | (Q8TD19) | NEK9_MOUSE | (Q8K1R7) |

RGC kinase: adenylyl cyclase class-4/guanylyl cyclase

| | | | | |
|---|---|---|---|---|
| GUCY2C | GUC2C_HUMAN | (P25092) | GUC2C_MOUSE | (Q3UWA6) |
| GUCY2D | GUC2D_HUMAN | (Q02846) | | |
| GUCY2F | GUC2F_HUMAN | (P51841) | GUC2F_MOUSE | (Q5SDA5) |
| Gucy2e | | | GUC2E_MOUSE | (P52785) |
| Gucy2g | | | GUC2G_MOUSE | (Q6TL19) |
| NPR1 | ANPRA_HUMAN | (P16066) | ANPRA_MOUSE | (P18293) |
| NPR2 | ANPRB_HUMAN | (P20594) | ANPRB_MOUSE | (Q6VVW5) |

STE Ser/Thr protein kinase family

| | | | | |
|---|---|---|---|---|
| MAP2K1 | MP2K1_HUMAN | (Q02750) | MP2K1_MOUSE | (P31938) |
| MAP2K2 | MP2K2_HUMAN | (P36507) | MP2K2_MOUSE | (Q63932) |
| MAP2K3 | MP2K3_HUMAN | (P46734) | MP2K3_MOUSE | (O09110) |
| MAP2K4 | MP2K4_HUMAN | (P45985) | MP2K4_MOUSE | (P47809) |
| MAP2K5 | MP2K5_HUMAN | (Q13163) | MP2K5_MOUSE | (Q9WVS7) |
| MAP2K6 | MP2K6_HUMAN | (P52564) | MP2K6_MOUSE | (P70236) |
| MAP2K7 | MP2K7_HUMAN | (O14733) | MP2K7_MOUSE | (Q8CE90) |
| MAP3K1 | M3K1_HUMAN | (Q13233) | M3K1_MOUSE | (P53349) |
| MAP3K10 | M3K10_HUMAN | (Q02779) | M3K10_MOUSE | (Q66L42) |
| MAP3K11 | M3K11_HUMAN | (Q16584) | M3K11_MOUSE | (Q80XI6) |
| MAP3K12 | M3K12_HUMAN | (Q12852) | M3K12_MOUSE | (Q60700) |
| MAP3K13 | M3K13_HUMAN | (O43283) | M3K13_MOUSE | (Q1HKZ5) |
| MAP3K14 | M3K14_HUMAN | (Q99558) | M3K14_MOUSE | (Q9WUL6) |
| MAP3K15 | M3K15_HUMAN | (Q6ZN16) | M3K15_MOUSE | (A2AQW0) |
| MAP3K19 | M3K19_HUMAN | (Q56UN5) | M3K19_MOUSE | (E9Q3S4) |
| MAP3K2 | M3K2_HUMAN | (Q9Y2U5) | M3K2_MOUSE | (Q61083) |
| MAP3K3 | M3K3_HUMAN | (Q99759) | M3K3_MOUSE | (Q61084) |
| MAP3K4 | M3K4_HUMAN | (Q9Y6R4) | M3K4_MOUSE | (O08648) |
| MAP3K5 | M3K5_HUMAN | (Q99683) | M3K5_MOUSE | (O35099) |
| MAP3K6 | M3K6_HUMAN | (O95382) | M3K6_MOUSE | (Q9WTR2) |
| MAP3K7 | M3K7_HUMAN | (O43318) | M3K7_MOUSE | (Q62073) |
| MAP3K8 | M3K8_HUMAN | (P41279) | M3K8_MOUSE | (Q07174) |
| MAP3K9 | M3K9_HUMAN | (P80192) | M3K9_MOUSE | (Q3U1V8) |
| MAP4K1 | M4K1_HUMAN | (Q92918) | M4K1_MOUSE | (P70218) |
| MAP4K2 | M4K2_HUMAN | (Q12851) | M4K2_MOUSE | (Q61161) |
| MAP4K3 | M4K3_HUMAN | (Q8IVH8) | M4K3_MOUSE | (Q99JP0) |
| MAP4K4 | M4K4_HUMAN | (O95819) | M4K4_MOUSE | (P97820) |
| MAP4K5 | M4K5_HUMAN | (Q9Y4K4) | M4K5_MOUSE | (Q8BPM2) |
| MINK1 | MINK1_HUMAN | (Q8N4C8) | MINK1_MOUSE | (Q9JM52) |
| MLK4 | M3KL4_HUMAN | (Q5TCX8) | M3KL4_MOUSE | (Q8VDG6) |
| MLTK | MLTK_HUMAN | (Q9NYL2) | MLTK_MOUSE | (Q9ESL4) |
| MST4 | MST4_HUMAN | (Q9P289) | MST4_MOUSE | (Q99JT2) |
| MYO3A | MYO3A_HUMAN | (Q8NEV4) | MYO3A_MOUSE | (Q8K3H5) |
| MYO3B | MYO3B_HUMAN | (Q8WXR4) | MYO3B_MOUSE | (Q1EG27) |
| NRK | NRK_HUMAN | (Q7Z2Y5) | NRK_MOUSE | (Q9R0G8) |
| OXSR1 | OXSR1_HUMAN | (O95747) | OXSR1_MOUSE | (Q6P9R2) |
| PAK1 | PAK1_HUMAN | (Q13153) | PAK1_MOUSE | (O88643) |
| PAK2 | PAK2_HUMAN | (Q13177) | PAK2_MOUSE | (Q8CIN4) |
| PAK3 | PAK3_HUMAN | (O75914) | PAK3_MOUSE | (Q61036) |
| PAK4 | PAK4_HUMAN | (O96013) | PAK4_MOUSE | (Q8BTW9) |
| PAK6 | PAK6_HUMAN | (Q9NQU5) | PAK6_MOUSE | (Q3ULB5) |
| PAK7 | PAK7_HUMAN | (Q9P286) | PAK7_MOUSE | (Q8C015) |
| SLK | SLK_HUMAN | (Q9H2G2) | SLK_MOUSE | (O54988) |
| STK10 | STK10_HUMAN | (O94804) | STK10_MOUSE | (O55098) |
| STK24 | STK24_HUMAN | (Q9Y6E0) | STK24_MOUSE | (Q99KH8) |
| STK25 | STK25_HUMAN | (O00506) | STK25_MOUSE | (Q9Z2W1) |
| STK3 | STK3_HUMAN | (Q13188) | STK3_MOUSE | (Q9JI10) |
| STK39 | STK39_HUMAN | (Q9UEW8) | STK39_MOUSE | (Q9Z1W9) |
| STK4 | STK4_HUMAN | (Q13043) | STK4_MOUSE | (Q9JI11) |
| STRADA | STRAA_HUMAN | (Q7RTN6) | STRAA_MOUSE | (Q3UUJ4) |
| STRADB | STRAB_HUMAN | (Q9C0K7) | STRAB_MOUSE | (Q8K4T3) |
| TAOK1 | TAOK1_HUMAN | (Q7L7X3) | TAOK1_MOUSE | (Q5F2E8) |
| TAOK2 | TAOK2_HUMAN | (Q9UL54) | TAOK2_MOUSE | (Q6ZQ29) |
| TAOK3 | TAOK3_HUMAN | (Q9H2K8) | TAOK3_MOUSE | (Q8BYC6) |
| TNIK | TNIK_HUMAN | (Q9UKE5) | TNIK_MOUSE | (P83510) |

TKL Ser/Thr protein kinase family

| | | | | |
|---|---|---|---|---|
| ACVR1 | ACVR1_HUMAN | (Q04771) | ACVR1_MOUSE | (P37172) |
| ACVR1B | ACV1B_HUMAN | (P36896) | ACV1B_MOUSE | (Q61271) |
| ACVR1C | ACV1C_HUMAN | (Q8NER5) | ACV1C_MOUSE | (Q8K348) |
| ACVR2A | AVR2A_HUMAN | (P27037) | AVR2A_MOUSE | (P27038) |
| ACVR2B | AVR2B_HUMAN | (Q13705) | AVR2B_MOUSE | (P27040) |
| ACVRL1 | ACVL1_HUMAN | (P37023) | ACVL1_MOUSE | (Q61288) |
| AMHR2 | AMHR2_HUMAN | (Q16671) | AMHR2_MOUSE | (Q8K592) |
| ANKK1 | ANKK1_HUMAN | (Q8NFD2) | ANKK1_MOUSE | (Q8BZ25) |
| ARAF | ARAF_HUMAN | (P10398) | ARAF_MOUSE | (P04627) |
| BMPR1A | BMR1A_HUMAN | (P36894) | BMR1A_MOUSE | (P36895) |
| BMPR1B | BMR1B_HUMAN | (O00238) | BMR1B_MOUSE | (P36898) |
| BMPR2 | BMPR2_HUMAN | (Q13873) | BMPR2_MOUSE | (O35607) |
| BRAF | BRAF_HUMAN | (P15056) | BRAF_MOUSE | (P28028) |
| ILK | ILK_HUMAN | (Q13418) | ILK_MOUSE | (O55222) |
| IRAK1 | IRAK1_HUMAN | (P51617) | IRAK1_MOUSE | (Q62406) |
| IRAK2 | IRAK2_HUMAN | (O43187) | IRAK2_MOUSE | (Q8CFA1) |
| IRAK3 | IRAK3_HUMAN | (Q9Y616) | IRAK3_MOUSE | (Q8K4B2) |
| IRAK4 | IRAK4_HUMAN | (Q9NWZ3) | IRAK4_MOUSE | (Q8R4K2) |
| KSR1 | KSR1_HUMAN | (Q8IVT5) | KSR1_MOUSE | (Q61097) |
| KSR2 | KSR2_HUMAN | (Q6VAB6) | KSR2_MOUSE | (Q3UVC0) |
| LIMK1 | LIMK1_HUMAN | (P53667) | LIMK1_MOUSE | (P53668) |
| LIMK2 | LIMK2_HUMAN | (P53671) | LIMK2_MOUSE | (O54785) |
| LRRK1 | LRRK1_HUMAN | (Q38SD2) | LRRK1_MOUSE | (Q3UHC2) |
| LRRK2 | LRRK2_HUMAN | (Q5S007) | LRRK2_MOUSE | (Q5S006) |
| RAF1 | RAF1_HUMAN | (P04049) | RAF1_MOUSE | (Q99N57) |
| RIPK1 | RIPK1_HUMAN | (Q13546) | RIPK1_MOUSE | (Q60855) |
| RIPK2 | RIPK2_HUMAN | (O43353) | RIPK2_MOUSE | (P58801) |
| RIPK3 | RIPK3_HUMAN | (Q9Y572) | RIPK3_MOUSE | (Q9QZL0) |
| RIPK4 | RIPK4_HUMAN | (P57078) | RIPK4_MOUSE | (Q9ERK0) |
| TESK1 | TESK1_HUMAN | (Q15569) | TESK1_MOUSE | (O70146) |
| TESK2 | TESK2_HUMAN | (Q96S53) | TESK2_MOUSE | (Q8VCT9) |
| TGFBR1 | TGFR1_HUMAN | (P36897) | TGFR1_MOUSE | (Q64729) |
| TGFBR2 | TGFR2_HUMAN | (P37173) | TGFR2_MOUSE | (Q62312) |
| TNNI3K | TNI3K_HUMAN | (Q59H18) | TNI3K_MOUSE | (Q5GIG6) |

Tyr protein kinase family

| | | | | |
|---|---|---|---|---|
| AATK | LMTK1_HUMAN | (Q6ZMQ8) | LMTK1_MOUSE | (Q80YE4) |
| ABL1 | ABL1_HUMAN | (P00519) | ABL1_MOUSE | (P00520) |
| ABL2 | ABL2_HUMAN | (P42684) | ABL2_MOUSE | (Q4JIM5) |
| ALK | ALK_HUMAN | (Q9UM73) | ALK_MOUSE | (P97793) |
| AXL | UFO_HUMAN | (P30530) | UFO_MOUSE | (Q00993) |
| BLK | BLK_HUMAN | (P51451) | BLK_MOUSE | (P16277) |
| BMX | BMX_HUMAN | (P51813) | BMX_MOUSE | (P97504) |
| BTK | BTK_HUMAN | (Q06187) | BTK_MOUSE | (P35991) |
| CSF1R | CSF1R_HUMAN | (P07333) | CSF1R_MOUSE | (P09581) |
| CSK | CSK_HUMAN | (P41240) | CSK_MOUSE | (P41241) |
| DDR1 | DDR1_HUMAN | (Q08345) | DDR1_MOUSE | (Q03146) |
| DDR2 | DDR2_HUMAN | (Q16832) | DDR2_MOUSE | (Q62371) |
| EGFR | EGFR_HUMAN | (P00533) | EGFR_MOUSE | (Q01279) |
| EPHA1 | EPHA1_HUMAN | (P21709) | EPHA1_MOUSE | (Q60750) |
| EPHA10 | EPHAA_HUMAN | (Q5JZY3) | EPHAA_MOUSE | (Q8BYG9) |
| EPHA2 | EPHA2_HUMAN | (P29317) | EPHA2_MOUSE | (Q03145) |
| EPHA3 | EPHA3_HUMAN | (P29320) | EPHA3_MOUSE | (P29319) |
| EPHA4 | EPHA4_HUMAN | (P54764) | EPHA4_MOUSE | (Q03137) |
| EPHA5 | EPHA5_HUMAN | (P54756) | EPHA5_MOUSE | (Q60629) |
| EPHA6 | EPHA6_HUMAN | (Q9UF33) | EPHA6_MOUSE | (Q62413) |
| EPHA7 | EPHA7_HUMAN | (Q15375) | EPHA7_MOUSE | (Q61772) |
| EPHA8 | EPHA8_HUMAN | (P29322) | EPHA8_MOUSE | (O09127) |
| EPHB1 | EPHB1_HUMAN | (P54762) | EPHB1_MOUSE | (Q8CBF3) |
| EPHB2 | EPHB2_HUMAN | (P29323) | EPHB2_MOUSE | (P54763) |
| EPHB3 | EPHB3_HUMAN | (P54753) | EPHB3_MOUSE | (P54754) |
| EPHB4 | EPHB4_HUMAN | (P54760) | EPHB4_MOUSE | (P54761) |
| EPHB6 | EPHB6_HUMAN | (O15197) | EPHB6_MOUSE | (O08644) |
| ERBB2 | ERBB2_HUMAN | (P04626) | ERBB2_MOUSE | (P70424) |
| ERBB3 | ERBB3_HUMAN | (P21860) | ERBB3_MOUSE | (Q61526) |
| ERBB4 | ERBB4_HUMAN | (Q15303) | ERBB4_MOUSE | (Q61527) |
| FER | FER_HUMAN | (P16591) | FER_MOUSE | (P70451) |
| FES | FES_HUMAN | (P07332) | FES_MOUSE | (P16879) |
| FGFR1 | FGFR1_HUMAN | (P11362) | FGFR1_MOUSE | (P16092) |
| FGFR2 | FGFR2_HUMAN | (P21802) | FGFR2_MOUSE | (P21803) |
| FGFR3 | FGFR3_HUMAN | (P22607) | FGFR3_MOUSE | (Q61851) |
| FGFR4 | FGFR4_HUMAN | (P22455) | FGFR4_MOUSE | (Q03142) |
| FGR | FGR_HUMAN | (P09769) | FGR_MOUSE | (P14234) |
| FLT1 | VGFR1_HUMAN | (P17948) | VGFR1_MOUSE | (P35969) |
| FLT3 | FLT3_HUMAN | (P36888) | FLT3_MOUSE | (Q00342) |
| FLT4 | VGFR3_HUMAN | (P35916) | VGFR3_MOUSE | (P35917) |
| FRK | FRK_HUMAN | (P42685) | FRK_MOUSE | (Q922K9) |
| FYN | FYN_HUMAN | (P06241) | FYN_MOUSE | (P39688) |
| HCK | HCK_HUMAN | (P08631) | HCK_MOUSE | (P08103) |
| IGF1R | IGF1R_HUMAN | (P08069) | IGF1R_MOUSE | (Q60751) |
| INSR | INSR_HUMAN | (P06213) | INSR_MOUSE | (P15208) |
| INSRR | INSRR_HUMAN | (P14616) | INSRR_MOUSE | (Q9WTL4) |
| ITK | ITK_HUMAN | (Q08881) | ITK_MOUSE | (Q03526) |
| JAK1 | JAK1_HUMAN | (P23458) | JAK1_MOUSE | (P52332) |
| JAK2 | JAK2_HUMAN | (O60674) | JAK2_MOUSE | (Q62120) |
| JAK3 | JAK3_HUMAN | (P52333) | JAK3_MOUSE | (Q62137) |
| KDR | VGFR2_HUMAN | (P35968) | VGFR2_MOUSE | (P35918) |
| KIT | KIT_HUMAN | (P10721) | KIT_MOUSE | (P05532) |
| LCK | LCK_HUMAN | (P06239) | LCK_MOUSE | (P06240) |
| LMTK2 | LMTK2_HUMAN | (Q8IWU2) | LMTK2_MOUSE | (Q3TYD6) |
| LMTK3 | LMTK3_HUMAN | (Q96Q04) | LMTK3_MOUSE | (Q5XJV6) |
| LTK | LTK_HUMAN | (P29376) | LTK_MOUSE | (P08923) |
| LYN | LYN_HUMAN | (P07948) | LYN_MOUSE | (P25911) |
| MATK | MATK_HUMAN | (P42679) | MATK_MOUSE | (P41242) |
| MERTK | MERTK_HUMAN | (Q12866) | MERTK_MOUSE | (Q60805) |
| MET | MET_HUMAN | (P08581) | MET_MOUSE | (P16056) |
| MST1R | RON_HUMAN | (Q04912) | RON_MOUSE | (Q62190) |
| MUSK | MUSK_HUMAN | (O15146) | MUSK_MOUSE | (Q61006) |
| NTRK1 | NTRK1_HUMAN | (P04629) | NTRK1_MOUSE | (Q3UFB7) |
| NTRK2 | NTRK2_HUMAN | (Q16620) | NTRK2_MOUSE | (P15209) |
| NTRK3 | NTRK3_HUMAN | (Q16288) | NTRK3_MOUSE | (Q6VNS1) |
| PDGFRA | PGFRA_HUMAN | (P16234) | PGFRA_MOUSE | (P26618) |
| PDGFRB | PGFRB_HUMAN | (P09619) | PGFRB_MOUSE | (P05622) |
| PTK2 | FAK1_HUMAN | (Q05397) | FAK1_MOUSE | (P34152) |
| PTK2B | FAK2_HUMAN | (Q14289) | FAK2_MOUSE | (Q9QVP9) |
| PTK6 | PTK6_HUMAN | (Q13882) | PTK6_MOUSE | (Q64434) |
| PTK7 | PTK7_HUMAN | (Q13308) | PTK7_MOUSE | (Q8BKG3) |
| RET | RET_HUMAN | (P07949) | RET_MOUSE | (P35546) |
| ROR1 | ROR1_HUMAN | (Q01973) | ROR1_MOUSE | (Q9Z139) |
| ROR2 | ROR2_HUMAN | (Q01974) | ROR2_MOUSE | (Q9Z138) |
| ROS1 | ROS1_HUMAN | (P08922) | ROS1_MOUSE | (Q78DX7) |
| RYK | RYK_HUMAN | (P34925) | RYK_MOUSE | (Q01887) |
| SRC | SRC_HUMAN | (P12931) | SRC_MOUSE | (P05480) |
| SRMS | SRMS_HUMAN | (Q9H3Y6) | SRMS_MOUSE | (Q62270) |
| STYK1 | STYK1_HUMAN | (Q6J9G0) | STYK1_MOUSE | (Q6J9G1) |
| SYK | KSYK_HUMAN | (P43405) | KSYK_MOUSE | (P48025) |
| Smok1 | | | SMOK1_MOUSE | (Q9QYZ4) |
| Smok4a | | | SMK4A_MOUSE | (A0JLX3) |
| Smoktcr | | | SMKTR_MOUSE | (A2KF29) |
| TEC | TEC_HUMAN | (P42680) | TEC_MOUSE | (P24604) |
| TEK | TIE2_HUMAN | (Q02763) | TIE2_MOUSE | (Q02858) |
| TIE1 | TIE1_HUMAN | (P35590) | TIE1_MOUSE | (Q06806) |
| TNK1 | TNK1_HUMAN | (Q13470) | TNK1_MOUSE | (Q99ML2) |
| TNK2 | ACK1_HUMAN | (Q07912) | ACK1_MOUSE | (O54967) |
| TXK | TXK_HUMAN | (P42681) | TXK_MOUSE | (P42682) |
| TYK2 | TYK2_HUMAN | (P29597) | TYK2_MOUSE | (Q9R117) |
| TYRO3 | TYRO3_HUMAN | (Q06418) | TYRO3_MOUSE | (P55144) |
| YES1 | YES_HUMAN | (P07947) | YES_MOUSE | (Q04736) |
| ZAP70 | ZAP70_HUMAN | (P43403) | ZAP70_MOUSE | (P43404) |

Other

| | | | | |
|---|---|---|---|---|
| AAK1 | AAK1_HUMAN | (Q2M2I8) | AAK1_MOUSE | (Q3UHJ0) |
| AURKA | AURKA_HUMAN | (O14965) | AURKA_MOUSE | (P97477) |
| AURKB | AURKB_HUMAN | (Q96GD4) | AURKB_MOUSE | (O70126) |
| AURKC | AURKC_HUMAN | (Q9UQB9) | AURKC_MOUSE | (O88445) |
| BMP2K | BMP2K_HUMAN | (Q9NSY1) | BMP2K_MOUSE | (Q91Z96) |
| BUB1 | BUB1_HUMAN | (O43683) | BUB1_MOUSE | (O08901) |
| BUB1B | BUB1B_HUMAN | (O60566) | BUB1B_MOUSE | (Q9Z1S0) |
| CAMKK1 | KKCC1_HUMAN | (Q8N5S9) | KKCC1_MOUSE | (Q8VBY2) |
| CAMKK2 | KKCC2_HUMAN | (Q96RR4) | KKCC2_MOUSE | (Q8C078) |
| CDC7 | CDC7_HUMAN | (O00311) | CDC7_MOUSE | (Q9Z0H0) |
| CHUK | IKKA_HUMAN | (O15111) | IKKA_MOUSE | (Q60680) |
| CSNK2A1 | CSK21_HUMAN | (P68400) | CSK21_MOUSE | (Q60737) |
| CSNK2A2 | CSK22_HUMAN | (P19784) | CSK22_MOUSE | (O54833) |
| DSTYK | DUSTY_HUMAN | (Q6XUX3) | DUSTY_MOUSE | (Q6XUX1) |
| EIF2AK1 | E2AK1_HUMAN | (Q9BQI3) | E2AK1_MOUSE | (Q9Z2R9) |
| EIF2AK2 | E2AK2_HUMAN | (P19525) | E2AK2_MOUSE | (Q03963) |
| EIF2AK3 | E2AK3_HUMAN | (Q9NZJ5) | E2AK3_MOUSE | (Q9Z2B5) |
| EIF2AK4 | E2AK4_HUMAN | (Q9P2K8) | E2AK4_MOUSE | (Q9QZ05) |
| ERN1 | ERN1_HUMAN | (O75460) | ERN1_MOUSE | (Q9EQY0) |
| ERN2 | ERN2_HUMAN | (Q76MJ5) | ERN2_MOUSE | (Q9Z2E3) |
| GAK | GAK_HUMAN | (O14976) | GAK_MOUSE | (Q99KY4) |
| GSG2 | HASP_HUMAN | (Q8TF76) | HASP_MOUSE | (Q9Z0R0) |
| IKBKB | IKKB_HUMAN | (O14920) | IKKB_MOUSE | (O88351) |
| IKBKE | IKKE_HUMAN | (Q14164) | IKKE_MOUSE | (Q9R0T8) |
| MLKL | MLKL_HUMAN | (Q8NB16) | MLKL_MOUSE | (Q9D2Y4) |
| MOS | MOS_HUMAN | (P00540) | MOS_MOUSE | (P00536) |
| NRBP1 | NRBP_HUMAN | (Q9UHY1) | NRBP_MOUSE | (Q99J45) |
| NRBP2 | NRBP2_HUMAN | (Q9NSY0) | NRBP2_MOUSE | (Q91V36) |
| PAN3 | PAN3_HUMAN | (Q58A45) | PAN3_MOUSE | (Q640Q5) |
| PBK | TOPK_HUMAN | (Q96KB5) | TOPK_MOUSE | (Q9JJ78) |
| PDIK1L | PDK1L_HUMAN | (Q8N165) | PDK1L_MOUSE | (Q8QZR7) |
| PEAK1 | PEAK1_HUMAN | (Q9H792) | PEAK1_MOUSE | (Q69Z38) |
| PIK3R4 | PI3R4_HUMAN | (Q99570) | PI3R4_MOUSE | (Q8VD65) |
| PINK1 | PINK1_HUMAN | (Q9BXM7) | PINK1_MOUSE | (Q99MQ3) |
| PKDCC | PKDCC_HUMAN | (Q504Y2) | PKDCC_MOUSE | (Q5RJI4) |
| PKMYT1 | PMYT1_HUMAN | (Q99640) | PMYT1_MOUSE | (Q9ESG9) |
| PLK1 | PLK1_HUMAN | (P53350) | PLK1_MOUSE | (Q07832) |
| PLK2 | PLK2_HUMAN | (Q9NYY3) | PLK2_MOUSE | (P53351) |
| PLK3 | PLK3_HUMAN | (Q9H4B4) | PLK3_MOUSE | (Q60806) |
| PLK4 | PLK4_HUMAN | (O00444) | PLK4_MOUSE | (Q64702) |
| PLK5 | PLK5_HUMAN | (Q496M5) | PLK5_MOUSE | (Q4FZD7) |
| PRKY | PRKY_HUMAN | (O43930) | | |
| PXK | PXK_HUMAN | (Q7Z7A4) | PXK_MOUSE | (Q8BX57) |
| RNASEL | RN5A_HUMAN | (Q05823) | RN5A_MOUSE | (Q05921) |
| RPS6KC1 | KS6C1_HUMAN | (Q96S38) | KS6C1_MOUSE | (Q8BLK9) |
| RPS6KL1 | RPKL1_HUMAN | (Q9Y6S9) | RPKL1_MOUSE | (Q8R2S1) |
| SBK1 | SBK1_HUMAN | (Q52WX2) | SBK1_MOUSE | (Q8QZX0) |
| SBK2 | SBK2_HUMAN | (P0C263) | SBK2_MOUSE | (POC5K1) |
| SCYL1 | NTKL_HUMAN | (Q96KG9) | NTKL_MOUSE | (Q9EQC5) |
| SCYL2 | SCYL2_HUMAN | (Q6P3W7) | SCYL2_MOUSE | (Q8CFE4) |
| SCYL3 | PACE1_HUMAN | (Q8IZE3) | PACE1_MOUSE | (Q9DBQ7) |
| SGK071 | SGK71_HUMAN | (Q8NE28) | SGK71_MOUSE | (Q80YS9) |
| SGK110 | SG110_HUMAN | (P0C264) | SG110_MOUSE | (POC5K0) |
| SGK196 | SG196_HUMAN | (Q9H5K3) | SG196_MOUSE | (Q3TUA9) |
| SGK223 | SG223_HUMAN | (Q86YV5) | SG223_MOUSE | (Q571I4) |
| SGK494 | SG494_HUMAN | (Q96LW2) | SG494_MOUSE | (Q5SYL1) |
| STK16 | STK16_HUMAN | (O75716) | STK16_MOUSE | (O88697) |
| STK31 | STK31_HUMAN | (Q9BXU1) | STK31_MOUSE | (Q99MW1) |
| STK32A | ST32A_HUMAN | (Q8WU08) | ST32A_MOUSE | (Q8BGW6) |
| STK32B | ST32B_HUMAN | (Q9NY57) | ST32B_MOUSE | (Q9JJX8) |
| STK32C | ST32C_HUMAN | (Q86UX6) | ST32C_MOUSE | (Q8QZV4) |
| STK35 | STK35_HUMAN | (Q8TDR2) | STK35_MOUSE | (Q80ZW0) |
| STK36 | STK36_HUMAN | (Q9NRP7) | STK36_MOUSE | (Q69ZM6) |
| TBCK | TBCK_HUMAN | (Q8TEA7) | TBCK_MOUSE | (Q8BM85) |
| TBK1 | TBK1_HUMAN | (Q9UHD2) | TBK1_MOUSE | (Q9WUN2) |
| TEX14 | TEX14_HUMAN | (Q8IWB6) | TEX14_MOUSE | (Q7M6U3) |
| TLK1 | TLK1_HUMAN | (Q9UKI8) | TLK1_MOUSE | (Q8C0V0) |
| TLK2 | TLK2_HUMAN | (Q86UE8) | TLK2_MOUSE | (O55047) |
| TP53RK | PRPK_HUMAN | (Q96S44) | PRPK_MOUSE | (Q99PW4) |
| TTK | TTK_HUMAN | (P33981) | TTK_MOUSE | (P35761) |
| UHMK1 | UHMK1_HUMAN | (Q8TAS1) | UHMK1_MOUSE | (P97343) |
| ULK1 | ULK1_HUMAN | (O75385) | ULK1_MOUSE | (O70405) |
| ULK2 | ULK2_HUMAN | (Q8IYT8) | ULK2_MOUSE | (Q9QY01) |
| ULK3 | ULK3_HUMAN | (Q6PHR2) | ULK3_MOUSE | (Q3U3Q1) |
| ULK4 | ULK4_HUMAN | (Q96C45) | ULK4_MOUSE | (Q3V129) |
| WEE1 | WEE1_HUMAN | (P30291) | WEE1_MOUSE | (P47810) |
| WEE2 | WEE2_HUMAN | (P0C1S8) | WEE2_MOUSE | (Q66JT0) |
| WNK1 | WNK1_HUMAN | (Q9H4A3) | WNK1_MOUSE | (P83741) |
| WNK2 | WNK2_HUMAN | (Q9Y3S1) | WNK2_MOUSE | (Q3UH66) |
| WNK3 | WNK3_HUMAN | (Q9BYP7) | WNK3_MOUSE | (Q80XP9) |
| WNK4 | WNK4_HUMAN | (Q96J92) | WNK4_MOUSE | (Q80UE6) |

Atypical: ADCK protein kinase family

| | | | | |
|---|---|---|---|---|
| ADCK1 | ADCK1_HUMAN | (Q 86TW2) | ADCK1_MOUSE | (Q9D0L4) |
| ADCK2 | ADCK2_HUMAN | (Q 7Z695) | ADCK2_MOUSE | (Q6NSR3) |
| ADCK3 | ADCK3_HUMAN | (Q 8NI60) | ADCK3_MOUSE | (Q60936) |
| ADCK4 | ADCK4_HUMAN | (Q 96D53) | ADCK4_MOUSE | (Q566J8) |
| ADCK5 | ADCK5_HUMAN | (Q 3MIX3) | ADCK5_MOUSE | (Q80V03) |

Atypical: Alpha-type protein kinase family

| | | | | |
|---|---|---|---|---|
| ALPK1 | ALPK1_HUMAN | (Q96QP1) | ALPK1_MOUSE | (Q9CXB8) |
| ALPK2 | ALPK2_HUMAN | (Q86TB3) | ALPK2_MOUSE | (Q91ZB0) |
| ALPK3 | ALPK3_HUMAN | (Q96L96) | ALPK3_MOUSE | (Q924C5) |
| EEF2K | EF2K_HUMAN | (O00418) | EF2K_MOUSE | (O08796) |
| TRPM6 | TRPM6_HUMAN | (Q9BX84) | TRPM6_MOUSE | (Q8CIR4) |
| TRPM7 | TRPM7_HUMAN | (Q96QT4) | TRPM7_MOUSE | (Q923J1) |

Atypical: FAST protein kinase family

| | | | | |
|---|---|---|---|---|
| FASTK | FASTK_HUMAN | (Q14296) | FASTK_MOUSE | (Q9JIX9) |

Atypical: PDK/BCKDK protein kinase family

| | | | | |
|---|---|---|---|---|
| BCKDK | BCKD_HUMAN | (O14874) | BCKD_MOUSE | (O55028) |
| PDK1 | PDK1_HUMAN | (Q15118) | PDK1_MOUSE | (Q8BFP9) |
| PDK2 | PDK2_HUMAN | (Q15119) | PDK2_MOUSE | (Q9JK42) |
| PDK3 | PDK3_HUMAN | (Q15120) | PDK3_MOUSE | (Q922H2) |
| PDK4 | PDK4_HUMAN | (Q16654) | PDK4_MOUSE | (O70571) |

Atypical: PI3/PI4-kinase family

| | | | | |
|---|---|---|---|---|
| ATM | ATM_HUMAN | (Q13315) | ATM_MOUSE | (Q62388) |
| ATR | ATR_HUMAN | (Q13535) | ATR_MOUSE | (Q9JKK8) |
| MTOR | MTOR_HUMAN | (P42345) | MTOR_MOUSE | (Q9JLN9) |
| PIK3CA | PK3CA_HUMAN | (P42336) | PK3CA_MOUSE | (P42337) |
| PIK3CG | PK3CG_HUMAN | (P48736) | PK3CG_MOUSE | (Q9JHG7) |
| PRKDC | PRKDC_HUMAN | (P78527) | PRKDC_MOUSE | (P97313) |
| SMG1 | SMG1_HUMAN | (Q96Q15) | SMG1_MOUSE | (Q8BKX6) |

Atypical: RIO-type Ser/Thr kinase family

| | | | | |
|---|---|---|---|---|
| RIOK1 | RIOK1_HUMAN | (Q9BRS2) | RIOK1_MOUSE | (Q922Q2) |
| RIOK2 | RIOK2_HUMAN | (Q9BVS4) | RIOK2_MOUSE | (Q9CQS5) |
| RIOK3 | RIOK3_HUMAN | (O14730) | RIOK3_MOUSE | (Q9DBU3) |

TABLE 2
LIST OF COMMERCIALLY AVAILABLE NEAR-INFRARED DYES
THAT CAN BE CONJUGATED TO PROTEIN KINASE PEPTIDE
SUBSTRATES AND USED IN THE PRESENT DISCLOSURE

| Dye | Exₘₐₓ (nm)* | Emₘₐₓ (nm)* |
|---|---|---|
| IRDye 800CW | 778 | 794 |
| IRDye 680RD | 680 | 694 |
| IRDye 680LT | 680 | 694 |
| IRDye 750 | 766 | 776 |
| IRDye 700DX | 680 | 687 |
| IRDye 800RS | 770 | 786 |
| IRDye 650 | 651 | 668 |
| IRDye 700 phosphoramidite | — | — |
| IRDye 800 phosphoramidite | — | — |
| Cy7.0 | 750 | 773 |
| Cy7.5 | 788 | 808 |
| CF ™ 680 | 681 | 698 |
| CF ™ 680R | 680 | 701 |
| CF ™ 750 | 755 | 777 |
| CF ™ 770 | 770 | 797 |
| CF ™ 790 | 784 | 806 |
| Cyto 750 | 748 | 772 |
| Cyto 770 | 769 | 796 |
| Cyto 780 | 783 | 800 |
| Cyto 840 | 844 | 884 |
| XenoLight CF680 | 681 | 698 |
| DyLight755 | 754 | 776 |

TABLE 3
RADIOACTIVE-FREE KINASE REACTION MIXTURE

| REAGENT | PURPOSE |
|---|---|
| 1 mM Adenosine triphosphate (ATP) | Phosphate donor molecule |
| 10 mM Magnesium Chloride (MgCl₂) | Enzyme cofactor |
| 10 mM Dithiothreitol (DTT) | Reducing agent preventing |
| | oxidation |
| 20 μM NIRF-Histone-H2B(29-35) | Non-radioactive peptide |
| | substrate for PKG-Iα and PKC |
| 20 mM Tris-HCl pH 7.4 | Buffering solution |
| *10 μM cyclic guanine monophosphate | Allosteric activator of all PKG |
| (cGMP) | isoforms |

*cGMP is an optional addition to the kinase reaction and is used to fully activate PKG in biological samples.

In one aspect of the disclosure, the radioactive-free kinase reaction mixture is able to support the addition of, but not limited to agents such as:
1.) Variations in ATP and MgCl₂ concentrations.
2.) Phosphatase inhibitors.
3.) Protease Inhibitors.
4.) Reducing agents.
5.) Detergents.
6.) Carrier agents.
7.) Different buffer systems.

TABLE 4
HOMOGENIZING BUFFER

| REAGENT | PURPOSE |
|---|---|
| 20 mM Potassium phosphate | Neutral pH buffering solution |
| (pH 7.0) | |
| 10 mM EDTA | Magnesium and calcium ion chelator |
| 0.5 mM isobutylmethylxanthine | Phosphodiesterase inhibitor |
| 10 mM DTT | Reducing agent preventing oxidation |

In one aspect of the disclosure, the homogenizing buffer is able to support the addition of, but not limited to agents such as:
1.) Variations in ATP and MgCl₂ concentrations.
2.) Phosphatase inhibitors.
3.) Protease Inhibitors.
4.) Reducing agents.
5.) Detergents.
6.) Carrier agents.
7.) Different buffer systems.

TABLE 5
RADIOACTIVE-FREE KINASE REACTION PROTOCOL

| | |
|---|---|
| 1. | Add biological/clinical sample to the radioactive kinase reaction mix |
| | containing 20 μM NIRF-Histone-H2B(29-35) or other NIRF-labeled |
| | substrate (depending of protein kinase activity being measured). |
| 2. | *Incubate sample for used time and 30° C. |
| 3. | Add 150 mM EDTA to terminate the kinase reaction. |
| 4. | Add a loading dye to visualize electrophoretic mobility in step 6. |
| 5. | Load the wells of a 1% agarose gel with each sample. |
| 6. | Connect the gel box anode and cathode to a power supply and |
| | separate the NIRF-Histone-H2B(29-35) at 75 V for 90 minutes. |
| 7. | Remove and scan gel using a near infrared imaging device |
| | (e.g. LI-COR Odyssey ®). |
| 8. | Quantify results using near infrared imaging device software |
| | (e.g. LI-COR Odyssey ® Application Software Version 2.1) |

*Time and temperature can vary, but a shorter reaction time can be used, such as 1 or 2 minutes, when using biological and clinical samples, in order to minimize loss of ATP, proteolysis of the measured protein kinase and phosphatase-catalyzed dephosphorylation that can occur during the kinase reaction. Colder temperatures can also be used for the kinase reaction to slow down the loss of ATP, the proteolysis and the dephosphorylation that can occur in biological and clinical samples.

TABLE 6
PARTIAL LIST OF KINASE INHIBITORS THAT ARE ABLE TO BE USED
AS ADDED COMPONENTS IN THE KINASE MEASUREMENTS

| Kinase Inhibitor | Chemical Abbreviation | Molecular Weight |
|---|---|---|
| (5Z)-7-Oxozeaenol | C₁₉H₂₂O₇ | 362.37 |
| 1 NA-PP1 (PP1 Analog) | C₁₉H₁₉N₅ | 317.4 |
| 1 NM-PP1 (PP1 Analog II) | C₂₀H₂₁N₅ | 331.4 |
| 2-Aminopurine | C₅H₅N₅ | 135.1267 |
| A-443654 | C₂₄H₂₃ON₅×2HCl | 470.39 |
| A-769662 | C₂₀H₁₂N₂O₃S | 360.39 |
| A-83-01 | C₂₅H₁₉N₅S | 421.52 |
| AG1296 (Tyrphostin) | C₁₆H₁₄N₂O₂ | 266.29 |
| AG1478 (Tyrphostin) | C₁₆H₁₄ClN₃O₂ | 315.75 |
| AG490 | C₁₇H₁₄N₂O₃ | 294.3 |
| AG879 (Tyrphostin) | C₁₈H₂₄N₂OS | 316.5 |
| Akt Inhibitor IV | C₃₁ H₂₇IN₄S | 614.6 |
| Akti 1/2 | C₃₄H₂₉N₇O | 551.6 |
| Alsterpaullone | C₁₆H₁₁N₃O₃ | 293.3 |
| Amgen TBK 1 inhibitor (Compound II) | C₂₈H₃₅N₇O₄ | 533.62 |
| Amlexanox | C₁₆H₁₄N₂O₄ | 298.293 |
| ARA014418 | C₁₂H₁₂N₄O₄S | 308.3 |
| AS601245 (JNK Inhibitor V) | C₂₀H₁₆N₆S | 372.5 |
| AX 20017 | C₁₃H₁₆N₂O₂S | 264.34 |
| AZD8055 | C₂₅H₃₁N₅O₄ | 465.5 |
| BAY 61-3606 | C₂₀H₁₈N₆O₃•xHCl•yH₂O | 390.4 |
| Bay 65-1942 | C₂₂H₂₆ClN₃O₄ | 431.91 |
| BAY-11-7082 | C₁₀H₉NO₂S | 207.2 |
| BAY439006 (Sorafenib, Nexavar) | C₂₁H₁₆ClF₃N₄O₃ | 464.8 |
| BI 2536 | C₂₈H₃₉N₇O₃ | 521.65 |
| BI-78D3 | C₁₃H₉N₅O₅S₂ | 379.37 |
| BI-D-1870 | C₁₉H₂₃N₅O₂F₂ | 391.42 |
| BIO (6-Bromoindirubin-3′-oxime) | C₁₆H₁₀BrN₃O₂ | 356.2 |
| BIRB-0796 (Doramapimod) | C₃₁H₃₇N₅O₃ | 527.66 |
| Blebbistatin | C₁₈H₁₆N₂O₂ | 292.3 |
| BML-258 (SK1-I) | C₁₇H₂₈ClNO₂ | 313.86 |
| BMS345541 (IKK Inhibitor III) | C₁₄H₁₇N₅ | 255.32 |
| BX-320 | C₂₃H₃₁BrN₈O₃ | 547.45 |
| BX-517 | C₁₅H₁₄N₄O₂ | 282.3 |
| BX-517 Analog (compound 7b) | C₁₉H₁₅N₅O₂ | 345.35 |
| BX-795 | C₂₃H₂₆IN₇O₂S | 591.47 |
| Caffeine | C₈H₁₀N₄O₂ | 194.19 |
| CAY10576 | C₂₂H₁₉N₃O₅S₂ | 469.5 |
| CCT250862 | | |
| CGP-57380 | C₁₁H₉FN₆ | 244.23 |
| CGP-57380 analog (SHN-093) | C₁₂H₁₁FN₆ | 258.25 |
| CGP-57380 analog (SHN-095) | C₁₃H₁₄N₆ | 254.29 |
| Chelerythrine Chloride | C₂₁H₁₈NO₄Cl | 383.8 |
| CHIR 99021 (CT 99021) | C₂₂H₁₈Cl₂N₈ | 465.34 |
| Chloroquine | C₁₈H₂₆ClN₃ | 319.87214 |
| CKI-7 | C₁₁H₁₂ClN₃O₂S•2HCl | 358.67 |
| Compound C (Dorsomorphin) | C₂₄H₂₅N₅O | 399.5 |
| Cot-Tpl2 Inhibitor Compound 10b | C₂₀H₁₅N₃OS | 345.42 |
| (Abbott) | | |
| Cot-Tpl2 Inhibitor Compound 38 | C₂₁H₁₂F₃N₅OS | 439.41 |
| (Abbott) | | |
| Cot-Tpl2 Inhibitor Compound 41 | C₂₁H₁₄N₆O₂S | 414.44 |
| (Abbott) | | |
| CP-690550 (Tasocitinib) | C₁₆H₂₀N₆O | 312.38 |
| Curcumin | C₂₁H₂₀O₆ | 368.4 |
| CZC-25146 | C₂₂H₂₅FN₆O₄S | 488.54 |
| D 4476 | C₂₃H₁₈N₄O₃ | 398.41 |
| Dimethyl fumarate | C₆H₈O₄ | 144.13 |
| EGCG (Epigallocatechin Gallate) | C₂₂H₁₈O₁₁ | 458.4 |
| ER-27319 | C₂₀H₂₂N₂O₅ | 370.4 |
| ETP 46464 | C₃₀H₂₂N₄O₂ | 470.52 |
| Febuxostat (TMX-67, Adenuric, | C₁₆H₁₆N₂O₃S | 316.37 |
| Uloric) | | |
| FMK (Caspase Inhibitor I) | C₂₂H₃₀FN₃O₇ | 467.5 |
| Fostamatinib | C23H26FN6O9P | 580.46 |
| GDC-0941 | C₂₃H₂₉N₇O₃S₂Cl₂ | 586.57 |
| Genentech LRRK2 inhibitor | C₁₉H₂₁F₄N₅O₃ | 443.4 |
| compound 18 (GNE-7915) | | |
| Genentech LRRK2 inhibitor | C₂₀H₂₁F₄N₅O₃ | 455.41 |
| compound 19 | | |
| Genistein | C₁₅H₁₀O₅ | 270.2 |
| GF 109203X (Go 6850) | C₂₅H₂₄N₄O₂ | 412.5 |
| Gleevec (Imatinib) | C₂₉H₃₁N₇O | 493.6 |
| Go 6976 | C₂₄H₁₈N₄O | 378.4 |
| Go 7874 | C₂₇H₂₆N₄O₄•HCl | 507 |
| GSK2334470 | C₂₅H₃₄N₈O | 462.59 |
| GSK2578215A | C₂₄H₁₈FN₃O₂ | 399.42 |
| GSK269962A | C₂₉H₃₀N₈O₅ | 570.61 |
| GSK429286 | C₂₁H₁₆F₄N₄O₂ | 432.37 |
| GSK461364 | C₂₇H₂₈F₃N₅O₂S | 543.6 |
| GSK650394A | C₂₅H₂₂N₂O₂ | 382.45 |
| GW441756 hydrochloride | C₁₇H₁₃N₃O•HCl | 311.77 |
| GW501516 | C₂₁H₁₈F₃NO₃S₂ | 453.5 |
| GW5074 (Raf1 Kinase Inhibitor I) | C₁₅H₈Br₂INO₂ | 520.9 |
| GW843682X (GSK-PLK1 | C₂₂H₁₈F₃N₃O₄S | 477.46 |
| Compound 2) | | |
| H-1152 (Rho Kinase Inhibitor) | C₁₆H₂₁N₃O₂S•2HCl | 392.3 |
| H-7, Dihydrochloride | C₁₄H₁₇N₃O₂S•2HCl | 364.3 |
| H-8, Dihydrochloride | C₁₂H₁₅N₃O₂S•2HCl | 338.3 |
| H-89, Dihydrochloride | C₂₀H₂₀BrN₃O₂S•2HCl | 519.3 |
| HA-1077, Dihydrochloride (Fasudil) | C₁₄H₁₇N₃O₂S•2HCl | 364.3 |
| HA-1100 (Hydroxyfasudil) | C₁₄H₁₇N₃O₃S•HCl | 343.83 |
| Harmaline | C₁₃H₁₄N₂O | 214.263 |
| Harmalol | C₁₂H₁₂N₂O•HCl•2H₂O | 272.73 |
| Harmane | C₁₂H₁₀N₂ | 182.22 |
| Harmine | C₁₃H₁₂N₂O | 212.25 |
| HG-10-102-01 | | |
| HG-9-91-01 | C₃₂H₃₈ClN₇O₃ | 604.14 |
| Hypothemycin | C₁₉H₂₂O₈ | 378.37 |
| IC261 | C₁₈H₁₇NO₄ | 311.3 |
| IKK Inhibitor VII | C₂₈H₂₉N₅OS | 483.6 |
| IKK-2 Inhibitor IV (TPCA-1) | C₁₂H₁₀FN₃O₂S | 279.3 |
| IKK-2 Inhibitor VIII | C₂₁H₂₄N₄O₂ | 364.4 |
| IKK-3 inhibitor IX | C₂₂H₁₉N₃O₅S₂ | 469.5 |
| IMD-0354 | C₁₅H₈ClF₆NO₂ | 383.67 |
| IPA-3 | C₂₀H₁₄O₂S₂ | 350.45 |
| IRAK 1-4 Inhibitor 1 | C₂₀H₂₁N₅O₄ | 395.41 |
| IRAK-4 kinase inhibitor a | C₂₂H₂₄N₄O₆ | 440.45 |
| IRAK-4 kinase inhibitor b | C₂₂H₂₄N₄O₆ | 440.45 |
| Ischemin | C₁₅H₁₇N₃O₄S | 335.38 |
| JNKIN7 | C₂₈H₂₇N₇O₂ | 493.56 |
| JNKIN8 | C₂₉H₂₉N₇O₂ | 507.59 |
| K252a | C₂₇H₂₁N₃O₅ | 467.5 |
| Kalopanaxsaponin A | C₄₁H₆₆O₁₂ | 750.96 |
| Kenpaullone | C₁₆H₁₁BrN₂O | 327.2 |
| KIN 112 | C₃₂H₃₆N₆O₄ | 568.67 |
| KN62 | C₃₈H₃₅N₅O₆S₂ | 721.9 |
| KN93 | C₂₆H₂₉CIN₂O₄S | 501 |
| KT5720 | C₃₂H₃₁N₃O₅ | 537.6 |
| KU 0063794 | C₂₅H₃₁N₅O₄ | 465.54 |
| KU 55933 | C₂₁H₁₇NO₃S₂ | 395.49 |
| LDN-193189 | C₂₅H₂₂N₆ | 406.49 |
| Leflunomide | C12H9F3N2O2 | 270.20727 |
| Lenalidomide (Revlimid) | C₁₃H₁₃N₃O₃ | 259.26 |
| LFM-A13 | C₁₁H₈Br₂N₂O₂ | 360 |
| LiCl | | 42.394 |
| LRRK2-IN1 | C₃₁H₃₈N₈O₃ | 570.69 |
| LY294002 | C₁₉H₁₇NO₃ | 307.4 |
| LY333531 (Ruboxistaurin) | C₂₈H₂₈IN₄O₃•CH₄O₃S | 582.73 |
| LY364947 | C₁₇H₁₂N₄ | 272.31 |
| Merck 7 (PDK1) | C₂₈H₂₂F₂N₄O₄ | 516.5 |
| MK-2206 | C₂₅H₂₁N₅O•2HCl | 480.39 |
| ML-7, Hydrochloride | C₁₅H₁₇IN₂O₂S•HCl | 452.7 |
| ML-9, Hydrochloride | C₁₅H₁₇ClN₂O₂S•HCl | 361.3 |
| MLN120B | C₁₉H₁₇Cl₃N₄O₂ | 439.72 |
| MLN4924 | C₂₁H₂₆ClN₅O₄S | 479.98 |
| MLN9708 | C₂₀H₂₃BCl₂N₂O₉ | 517.12 |
| MRT199665 | C₂₈H₃₁N₅O₂•ClH | 506.04 |
| MRT67307 | C₂₆H₃₆N₆O₂ | 464.6 |
| MSC 2032964A | C₁₆H₁₃F₃N₆O | 362.31 |
| Necrostatin-1 (Nec-1) | C₁₃H₁₃N₃OS | 259.33 |
| Nectrostatin-1 (Nec-1 inactive | C₁₂H₁₁N₃OS | 245.3 |
| analog) | | |
| NG-25 | C₂₉H₃₀F₃N₅O₂ | 537.58 |
| Novartis 12a (PKD1) | C₂₃H₂₉F₃N₆O₃ | 494.51 |
| NVP-BEZ 235 | C₃₀H₂₅Cl₂N₅O | 542.46 |
| OSU-03012 | C₂₆H₁₉F₃N₄O | 460.45 |
| OTSSP167 | C25H28Cl2N4O2 | 487.42 |
| Parthenolide | C₁₅H₂₀O₃ | 248.32 |
| PCI-32765 (Ibrutinib) | C₂₅H₂₄N₆O₂ | 440.5 |
| PD 0325901 | C₁₆H₁₄F₃IN₂O₄ | 482.19 |
| PD 0325901-Cl (CI-1055-R) | C₁₆H₁₄O₄N₂F₂ClI | 498.65 |
| PD 0325901-Cl (CI-1055-Racemic | C₁₆H₁₄O₄N₂F₂ClI | 498.65 |
| mix of isomers) | | |
| PD 0325901-Cl (CI-1055-S) | C₁₆H₁₄O₄N₂F₂ClI | 498.65 |
| PD 0332991 | C₂₄H₃₀ClN₇O₂ | 483.99 |
| PD 184352 (CI-1040) | C₁₇H₁₄ClF₂IN₂O₂ | 478.66 |
| PF3644022 | C₂₁H₁₈N₄OS | 374.46 |
| PF4708671 | C₁₉H₂₁F₃N₆ | 390.41 |
| PI-103 | C₁₉H₁₆N₄O₃ | 348.36 |
| Piceatannol | C₁₄H₁₂O₄ | 244.2 |
| Pirfenidone | C₁₂H₁₁NO | 185.23 |
| PKR Inhibitor | C₁₃H₈N₄OS | 268.3 |
| PLX 4032 (Vemurafenib) | C₂₃H₁₈ClF₂N₃O₃S | 489.92 |
| PLX 4720 (Raf Kinase Inhibitor V) | C₁₇H₁₄ClF₂N₃O₃S | 413.83 |
| PP1 | C₁₆H₁₉N₅ | 281.36 |
| PP2 | C₁₅H₁₆ClN₅ | 301.77 |
| PP242 | C₁₆H₁₆N₆O | 308.3 |
| PP3 | C₁₁H₉N₅ | 211.22 |
| Princeton's TrkA inhibitor compound | C₂₄H₂₄BrF₃N₆O₄S₂ | 661.52 |
| 20h | | |
| PS-1145 | C₁₇H₁₁ClN₄O | 322.75 |
| Purvalanol A | C₁₉H₂₅ClN₆O | 388.9 |
| Purvalanol B | C₂₀H₂₅ClN₆O₃ | 432.9 |
| Quercetagetin | C₁₅H₁₀O₈ | 318.3 |
| R-406 | C₂₃H₂₄FN₅O₅ | 469.46 |
| Rapamycin | C₅₁H₇₉NO₁₃ | 914.2 |
| Resveratrol | C₁₄H₁₂O₃ | 228.2 |
| Rigel TRAF6 Inhibitor | C₁₆H₁₂ClF₄N₅O₃S | 465.81 |
| RO-31-8220 | C₂₅H₂₃N₅O₂S•CH₄O₃S | 553.7 |
| Roscovitine | C₁₉H₂₆N₆O | 354.5 |
| Rottlerin | C₃₀H₂₈O₈ | 516.5 |
| Ruxolitinib (INCB18424) | C₁₇H₁₈N₆ | 306.37 |
| Saracatinib (AZD0530) | C₂₇H₃₂ClN₅O₅ | 542.03 |
| SB-202190 | C₂₀H₁₄FN₃O | 331.3 |
| SB-203580 | C₂₁H₁₆N₃OSF | 377.4 |
| SB-216763 | C₁₉H₁₂Cl₂N₂O₂ | 371.22 |
| SB-415286 | C₁₆H₁₀ClN₃O₅ | 359.73 |
| SB-431542 | C₂₂H₁₆N₄O₃•xH₂O | 384.39 |
| SB-505124 | C₂₀H₂₁N₃O₂•xHCl•yH₂O | 335.4 |
| SB-590885 | C₂₇H₂₇N₅O₂ | 453.54 |
| SB-747651A | C₁₆H₂₂N₈O | 342.4 |
| SC514 (IKK-2 Inhibitor) | C₉H₈N₂OS₂ | 224.3 |
| SL-327 (MEK1-2 Inhibitor) | C₁₆H₁₂F₃N₃S | 335.4 |
| SL0101-1 | C₂₅H₂₄O₁₂ | 516.46 |
| Sodium salicylate | HOC₆H₄COONa | 160.1 |
| Sodium valproate | C₈H₁₅NaO₂ | 166.19 |
| SP600125 (JNK Inhibitor II, SAPK | C₁₄H₈N₂O | 220.2 |
| Inhibitor II) | | |
| Sphingosine Kinase Inhibitor (SKI | C₁₅H₁₁ClN₂OS | 339.2 |
| II) | | |
| SRC Inhibitor 1 | C₂₂H₁₉N₃O₃ | 373.4 |
| SRT 1720 | C₂₇H₂₄F₃N₇O₃S | 583.59 |
| Staurosporine | C₂₈H₂₆N₄O₃ | 466.54 |
| STO 609 | C₁₉H₁₀N₂O₃•CH₃CO₂H | 374.4 |
| STOCK1S-50699 | C₂₉H₃₁N₂BrS₂ | 551.60254 |
| STOCK2S-26016 | C₂₀H₁₉N₃O₂ | 333.38976 |
| SU-5416 (VEGFR2 Kinase Inhibitor | C₁₅H₁₄N₂O | 238.3 |
| III) | | |
| SU-6656 | C₁₉H₂₁N₃O₃S | 371.5 |
| SU-6668 | C₁₈H₁₈N₂O₃ | 310.35 |
| Sulfasalazine | C₁₈H₁₄N₄O₅S | 398.39 |
| Sunitinib (SU-11248) | C₂₂H₂₇FN₄O₂ | 398.47 |
| SYK Inhibitor I | C₁₈H₁₅N₃O₃S | 353.4 |
| SYK Inhibitor II | C₁₄H₁₅F₃N₆O•2HCl•2H₂O | 449.3 |
| SYK Inhibitor III | C₉H₇NO₄ | 193.2 |
| SYK Inhibitor IV (BAY 61-3606) | C₂₀H₁₈N₆O₃•HCl•H₂O | 444.9 |
| TAE684 | C₃₀H₄₀ClN₇O₃S | 614.22 |
| Tenovin-6 | C25H34N4O2S | 454.6 |
| TG003 | C₁₃H₁₅NO₂S | 249.33 |
| Torin1 | C₃₅H₂₈F₃N₅O₂ | 607.62 |
| TTT-3002 | | |
| Ubiquitin E1 Inhibitor (PYR-41) | C₁₇H₁₃N₃O₇•3H₂O | 425.4 |
| UCN-01 | C₂₈H₂₆N₄O₄ | 482.5 |
| Uo126 | C₁₈H₁₆N₆S₂ | 380.48 |
| VX-680 | C₂₃H₂₈N₈OS | 464.59 |
| VX-745 | C₁₉H₉Cl₂F₂N₃OS | 436.26 |
| Withaferin A | C₂₈H₃₈O₆ | 470.6 |
| Wortmannin (KY 12420) | C₂₃H₂₄O₈ | 428.4 |
| Wyeth PDK1 Inhibitor Compound 1 | C₁₉H₂₂Cl₄N₄O₂ | 480.21 |
| Y-27632 | C₁₄H₂₁N₃O•2HCl•H₂O | 338.3 |
| Zearalenone | C₁₈H₂₂O₅ | 318.4 |
| ZM 336372 | C₂₃H₂₃N₃O₃ | 389.4 |

TABLE 7
PARTIAL LIST OF KINASE SUBSTRATE PEPTIDES THAT CAN BE
LABELED BY NIRF DYES AND USED IN THE KINASE MEASUREMENT

| | | | | Phosphate |
|---|---|---|---|---|
| | | | | Acceptor/ |
| Sequence | ID | Protein Names | Region | Kinases |
| DFRTRESTAKKIK | RGS1_HUMAN | REGULATOR OF G- | D139 | T146/PKC |
| | | PROTEIN SIGNALING | K151 | (potential) |
| | | 1 (RGS1) (EARLY | | |
| | | RESPONSE PROTEIN | | |
| | | 1R20) (B-CELL | | |
| | | ACTIVATION | | |
| | | PROTEIN | | |
| | | BL34). | | |
| ERSKTVTSFYN | BCKD_HUMAN | [3-METHYL-2- | E45 S57 | S52 |
| QS | | OXOBUTANOATE | | (autophosphorylation) |
| | | DEHYDROGENASE | | |
| | | (LIPOAMIDE)] | | |
| | | KINASE, | | |
| | | MITOCHONDRIAL | | |
| | | (EC 2.7.1.115) | | |
| | | (BRANCHED-CHAIN | | |
| | | ALPHA-KETOACID | | |
| | | DEHYDROGENASE | | |
| | | KINASE) | | |
| | | (BCKDHKIN) (BCKD- | | |
| | | KINASE). | | |
| LTLWTSDSAGE | B140-A | 14-3-3 PROTEIN TAU | L225 | S 232/CKI |
| EC | | (14-3-3 PROTEIN | C237 | alpha |
| | | THETA) (HS1 | | |
| | | PROTEIN) | | |
| FYYEILNSPEKAC | 143Z_HUMAN | 14-3-3 PROTEIN | F177 | S184/ |
| | | ZETA/DELTA | C189 | proline- |
| | | (PROTEIN KINASE C | | directed |
| | | INHIBITOR PROTEIN- | | kinases |
| | | 1) (KCIP-1) (FACTOR | | |
| | | ACTIVATING | | |
| | | EXOENZYME S) | | |
| | | (FAS). | | |
| YDVSRMYVDPS | PIG2_HUMAN | 1- | Y753 | Y753/Lck, |
| EI | | PHOSPHATIDYLINOSITOL- | I765 | Fyn, Lyn; |
| | | 4,5- | | Y759/ |
| | | BISPHOSPHATE | | Lck, Fyn, |
| | | PHOSPHODIESTERASE | | Lyn |
| | | GAMMA (EC | | |
| | | 3.1.4.11) (PLC- | | |
| | | GAMMA- | | |
| | | 2) (PHOSPHOLIPASE | | |
| | | C-GAMMA-2) (PLC- | | |
| | | IV) | | |
| RDINSLYDVSR | PIG2_HUMAN | 1- | R 747 | Y753/Lck, |
| MY | | PHOSPHATIDYLINOSITOL- | Y759 | Fyn, Lyn; |
| | | 4,5- | | Y759/ |
| | | BISPHOSPHATE | | Lck, Fyn, |
| | | PHOSPHODIESTERASE | | Lyn |
| | | GAMMA (EC | | |
| | | 3.1.4.11) (PLC- | | |
| | | GAMMA- | | |
| | | 2) (PHOSPHOLIPASE | | |
| | | C-GAMMA-2) (PLC- | | |
| | | IV) | | |
| EGSFESRYQQPFE | PIP4_HUMAN | 1- | E1246 | S 1248/ |
| | | PHOSPHATIDYLINOSITOL- | E1258 | PKC, PKA; |
| | | 4,5- | | Y 1253 |
| | | BISPHOSPHATE | | |
| | | PHOSPHODIESTERASE | | |
| | | GAMMA 1 (EC | | |
| | | 3.1.4.11) (PLC- | | |
| | | GAMMA-1) | | |
| | | (PHOSPHOLIPASE C- | | |
| | | GAMMA-1) (PLC-II) | | |
| | | (PLC- | | |
| | | 148). | | |
| IGTAEPDYGAL | PIP4_HUMAN | 1- | I 764 | Y771/Syk |
| YE | | PHOSPHATIDYLINOSITOL- | E776 | |
| | | 4,5- | | |
| | | BISPHOSPHATE | | |
| | | PHOSPHODIESTERASE | | |
| | | GAMMA 1 (EC | | |
| | | 3.1.4.11) (PLC- | | |
| | | GAMMA-1) | | |
| | | (PHOSPHOLIPASE C- | | |
| | | GAMMA-1) (PLC-II) | | |
| | | (PLC- | | |
| | | 148). | | |
| DDSSAYRSVDE | ODBA_HUMAN | 2-OXOISOVALERATE | D338 | S345 |
| VN | | DEHYDROGENASE | N350 | |
| | | ALPHA SUBUNIT, | | |
| | | MITOCHONDRIAL | | |
| | | (EC 1.2.4.4) | | |
| | | (BRANCHED-CHAIN | | |
| | | ALPHA- | | |
| | | KETO ACID | | |
| | | DEHYDROGENASE | | |
| | | COMPONENT ALPHA | | |
| | | CHAIN (E1)) (BCKDH | | |
| | | E1-ALPHA). | | |
| TYRIGHHSTSDDS | ODBA_HUMAN | 2-OXOISOVALERATE | T328 | S335 |
| | | DEHYDROGENASE | S340 | |
| | | ALPHA SUBUNIT, | | |
| | | MITOCHONDRIAL | | |
| | | (EC 1.2.4.4) | | |
| | | (BRANCHED-CHAIN | | |
| | | ALPHA- | | |
| | | KETO ACID | | |
| | | DEHYDROGENASE | | |
| | | COMPONENT ALPHA | | |
| | | CHAIN (E1)) (BCKDH | | |
| | | E1-ALPHA). | | |
| RLSSLRASTSKSE | B043-C | 40S RIBOSOMAL | R233 | S235/ |
| | | PROTEIN S6 | E245 | S6/H4 |
| | | (PHOSPHOPROTEIN | | kinase; |
| | | NP33) | | S236/ |
| | | | | S6/H4 |
| | | | | kinase; |
| | | | | S240/ |
| | | | | S6/H4 |
| | | | | kinase; |
| | | | | S242/ |
| | | | | S6/H4 |
| | | | | kinase; |
| | | | | S244 |
| IAKRRRLSSLRAS | RS6_HUMAN | 40S RIBOSOMAL | I228 | S235/ |
| | | PROTEIN S6 | S240 | S6/H4 |
| | | (PHOSPHOPROTEIN | | kinase; |
| | | NP33). | | S236/ |
| | | | | S6/H4 |
| | | | | kinase; |
| | | | | S240/ |
| | | | | S6/H4 |
| | | | | kinase |
| LSSLRASTSKSES | RS6_HUMAN | 40S RIBOSOMAL | L234 | S235/ |
| | | PROTEIN S6 | S246 | S6/H4 |
| | | (PHOSPHOPROTEIN | | kinase; |
| | | NP33). | | S236/ |
| | | | | S6/H4 |
| | | | | kinase; |
| | | | | S240/ |
| | | | | S6/H4 |
| | | | | kinase; |
| | | | | S242/ |
| | | | | S6/H4 |
| | | | | kinase; |
| | | | | S244 |
| PPGDYSTTPGGTL | Q13541 | 4E-BINDING | P30 L42 | T37/ |
| | | PROTEIN 1 | | RAFT1/FRAP/ |
| | | (EUKARYOTIC | | mTOR |
| | | TRANSLATION | | |
| | | INITIATION FACTOR | | |
| | | 4BINDING PROTEIN | | |
| | | 1) | | |
| SDGEFLRTSCGSP | AAK1_HUMAN | 5′-AMP-ACTIVATED | S167 | T174 |
| | | PROTEIN KINASE, | P179 | (autophosphorylation) |
| | | CATALYTIC ALPHA-1 | | |
| | | CHAIN (EC 2.7.1.—) | | |
| | | (AMPK ALPHA-1 | | |
| | | CHAIN) | | |
| | | (FRAGMENT). | | |
| EEDTDEDSDNEIH | PP65_HCMVT | 64 KDA LOWER | E455 | S462 |
| | | MATRIX | H467 | |
| | | PHOSPHOPROTEIN | | |
| | | (PP64) (GP64). | | |
| PLASPEPTKKPRI | F26P_HUMAN | 6PF-2-K/FRU-2,6- | P464 | T471/PKC |
| | | P2ASE | I476 | |
| | | BRAIN/PLACENTA- | | |
| | | TYPE ISOZYME | | |
| | | [INCLUDES: 6- | | |
| | | PHOSPHOFRUCTO-2- | | |
| | | KINASE (EC | | |
| | | 2.7.1.105); | | |
| | | FRUCTOSE-2,6- | | |
| | | BISPHOSPHATASE | | |
| | | (EC 3.1.3.46)]. | | |
| NPLMRRNSVTP | F26P_HUMAN | 6PF-2-K/FRU-2,6- | N454 | S461/PKA |
| LA | | P2ASE | A466 | |
| | | BRAIN/PLACENTA- | | |
| | | TYPE ISOZYME | | |
| | | [INCLUDES: 6- | | |
| | | PHOSPHOFRUCTO-2- | | |
| | | KINASE (EC | | |
| | | 2.7.1.105); | | |
| | | FRUCTOSE-2,6- | | |
| | | BISPHOSPHATASE | | |
| | | (EC 3.1.3.46)]. | | |
| RLQRRRGSSIPQF | F26L_HUMAN | 6PF-2-K/FRU-2,6- | R26 F38 | S33/PKA |
| | | P2ASE LIVER | | |
| | | ISOZYME | | |
| | | [INCLUDES: 6- | | |
| | | PHOSPHOFRUCTO-2- | | |
| | | KINASE (EC | | |
| | | 2.7.1.105); | | |
| | | FRUCTOSE- | | |
| | | 2,6- | | |
| | | BISPHOSPHATASE | | |
| | | (EC 3.1.3.46)]. | | |
| LNVAAVNTHRD | F264_HUMAN | 6-PHOSPHOFRUCTO- | L436 | T443/PKC |
| RP | | 2- | P448 | |
| | | KINASE/FRUCTOSE- | | |
| | | 2,6-BIPHOSPHATASE | | |
| | | 4 (6PF-2-K/FRU-2,6- | | |
| | | P2ASE TESTIS-TYPE | | |
| | | ISOZYME) | | |
| | | [INCLUDES: 6- | | |
| | | PHOSPHOFRUCTO-2- | | |
| | | KINASE (EC | | |
| | | 2.7.1.105); | | |
| | | FRUCTOSE-2,6- | | |
| | | BISPHOSPHATASE | | |
| | | (EC 3.1.3.46)]. | | |
| LEHVTRRTLSM | B063-A | 6- | L766 | T772/PKA |
| DK | | PHOSPHOFRUCTOKINASE, | K778 | |
| | | LIVER TYPE | | |
| WGRGTDEYFIR | ACHB_HUMAN | ACETYLCHOLINE | W383 | Y390/ |
| KP | | RECEPTOR PROTEIN, | P395 | TYR- |
| | | BETA CHAIN. | | Kinasen |
| YISKAEEYFLLKS | ACHD_HUMAN | ACETYLCHOLINE | Y383 | Y390/ |
| | | RECEPTOR PROTEIN, | S395 | TYR- |
| | | DELTA CHAIN. | | Kinasen |
| IPTLNRMSFSSNL | COA1_HUMAN | ACETYL-COA | I1194 | S1201 |
| | | CARBOXYLASE 1 | L1206 | |
| | | (EC 6.4.1.2) (ACC- | | |
| | | ALPHA) | | |
| | | [INCLUDES: BIOTIN | | |
| | | CARBOXYLASE (EC | | |
| | | 6.3.4.14)]. | | |
| LALHIRSSWSGLH | COA1_HUMAN | ACETYL-COA | L71 H83 | S 78; S80/ |
| | | CARBOXYLASE 1 | | AMPK |
| | | (EC 6.4.1.2) (ACC- | | |
| | | ALPHA) [INCLUDES: | | |
| | | BIOTIN | | |
| | | CARBOXYLASE (EC | | |
| | | 6.3.4.14)]. | | |
| RVPTMRPSMSG | COA2_HUMAN | ACETYL-COA | R212 | S219; S221/ |
| LH | | CARBOXYLASE 2 | H224 | AMPK |
| | | (EC 6.4.1.2) (ACC- | | |
| | | BETA) [INCLUDES: | | |
| | | BIOTIN | | |
| | | CARBOXYLASE (EC | | |
| | | 6.3.4.14)]. | | |
| PSFLRAPSWFDTG | CRAB_HUMAN | ALPHA CRYSTALLIN | P52 G64 | S53; S59/ |
| | | B CHAIN (ALPHA(B)- | | MAP |
| | | CRYSTALLIN) | | kinase, |
| | | (ROSENTHAL FIBER | | MAPKAP |
| | | COMPONENT). | | |
| FPTSTSLSPFYLR | CRAB_HUMAN | ALPHA CRYSTALLIN | F38 R50 | S43; S45/ |
| | | B CHAIN (ALPHA(B)- | | MAPkinase, |
| | | CRYSTALLIN) | | MAPKAP |
| | | (ROSENTHAL FIBER | | |
| | | COMPONENT). | | |
| SGASTGIYEALEL | A003-A | ALPHA ENOLASE | S36 L48 | Y43/v-Src; |
| | | | | Y43/gag- |
| | | | | fps; Y43/ |
| | | | | p140gag-fps |
| YVVAKRESRGL | A1AA_HUMAN | ALPHA-1A | Y208 | S215/PKA |
| KS | | ADRENERGIC | S220 | |
| | | RECEPTOR (ALPHA | | |
| | | 1A- | | |
| | | ADRENOCEPTOR) | | |
| | | (ALPHA-1C | | |
| | | ADRENERGIC | | |
| | | RECEPTOR). | | |
| ESSISSSSEEMSL | CAS1_HUMAN | ALPHA-S1 CASEIN. | E84 L96 | S86; S88; |
| | | | | S89; S90; |
| | | | | S91 |
| | | | | (potential) |
| EKMESSISSSSEE | CAS1_HUMAN | ALPHA-S1 CASEIN. | E81 E93 | S86; S88; |
| | | | | S89; S90; |
| | | | | S91 |
| | | | | (potential) |
| AEPEKMESSISSS | CAS1_HUMAN | ALPHA-S1 CASEIN. | A78 S90 | S86; S88; |
| | | | | S89; S90 |
| | | | | (potential) |
| HPGYINFSYEVLT | AMEX_HUMAN | AMELOGENIN, X | H25 T37 | S32 |
| | | ISOFORM. | | |
| AGERRKGTDVN | B088-D | ANNEXIN I | A208 | T215/ |
| VF | | (LIPOCORTIN I) | F220 | adenosine |
| | | (CALPACTIN II) | | cyclic |
| | | | | 3′,5′- |
| | | | | phosphate |
| | | | | dependent |
| | | | | protein |
| | | | | kinase |
| EEQEYVQTVKS | B088-C | ANNEXIN I | E16 K28 | Y20/ |
| SK | | (LIPOCORTIN I) | | EGFR; T23; |
| | | (CALPACTIN II) | | S26/PKC |
| EYVQTVKSSKG | B088-A | ANNEXIN I | E19 P31 | Y20/ |
| GP | | (LIPOCORTIN I) | | EGFR; T23; |
| | | (CALPACTIN II) | | S26/PKC |
| IENEEQEYVQTVK | B088-E | ANNEXIN I | I13 K25 | Y20/ |
| | | (LIPOCORTIN I) | | EGFR; T23 |
| | | (CALPACTIN II) | | |
| HSTPPSAYGSVKA | ANX2_HUMAN | ANNEXIN II | H16 | Y23/ |
| | | (LIPOCORTIN II) | A28 | pp60v-scr: |
| | | (CALPACTIN I | | S25/PKC |
| | | HEAVY CHAIN) | | |
| | | (CHROMOBINDIN 8) | | |
| | | (P36) (PROTEIN I) | | |
| | | (PLACENTAL | | |
| | | ANTICOAGULANT | | |
| | | PROTEIN IV) (PAP- | | |
| | | IV). | | |
| FQDIQQLSSEEND | LECI_HUMAN | ASIALOGLYCOPROTEIN | F5 D17 | S12/PKC |
| | | RECEPTOR 2 | | |
| | | (HEPATIC LECTIN | | |
| | | H2) (ASGP-R) | | |
| | | (ASGPR). | | |
| KDIIRQPSEEEII | PE15_HUMAN | ASTROCYTIC | K109 | S116/PKB |
| | | PHOSPHOPROTEIN | I121 | |
| | | PEA-15 (PED). | | |
| TKLTRIPSAKKYK | PE15_HUMAN | ASTROCYTIC | T97 | S104/PKC |
| | | PHOSPHOPROTEIN | K109 | |
| | | PEA-15 (PED). | | |
| PAPSRTASFYESM | ACLY_HUMAN | ATP-CITRATE (PRO- | | |
| | | S-)-LYASE (EC 4.1.3.8) | | |
| | | (CITRATE | | |
| | | CLEAVAGE | | |
| | | ENZYME). | | |
| REEAIKFSEEQRF | KU86_HUMAN | ATP-DEPENDENT | R642 | S649/ |
| | | DNA HELICASE II, 80 KDA | F654 | Nuclear |
| | | SUBUNIT | | Kinase NII |
| | | (LUPUS KU | | |
| | | AUTOANTIGEN | | |
| | | PROTEIN P86) (KU86) | | |
| | | (KU80) (86 KDA | | |
| | | SUBUNIT OF KU | | |
| | | ANTIGEN) | | |
| | | (THYROID-LUPUS | | |
| | | AUTOANTIGEN) | | |
| | | (TLAA) (CTC BOX | | |
| | | BINDING FACTOR 85 KDA | | |
| | | SUBU IT) (CTCBF) | | |
| | | (CTC85) (NUCLEAR | | |
| | | FACTOR IV) (DNA- | | |
| | | REPAIR PROTEIN | | |
| | | XRCC5). | | |
| ATDYHTTSHPG | B3AT_HUMAN | BAND 3 ANION | A43 | Y46/CK; |
| TH | | TRANSPORT | H55 | S50/CK |
| | | PROTEIN (ANION | | |
| | | EXCHANGE PROTEIN | | |
| | | 1) (AE 1). | | |
| EDPDIPESQMEEP | B3AT_HUMAN | BAND 3 ANION | E22 P34 | S29/CK |
| | | TRANSPORT | | |
| | | PROTEIN (ANION | | |
| | | EXCHANGE PROTEIN | | |
| | | 1) (AE 1). | | |
| TEATATDYHTT | B3AT_HUMAN | BAND 3 ANION | T39 H51 | Y46/CK; |
| SH | | TRANSPORT | | S50/CK |
| | | PROTEIN (ANION | | |
| | | EXCHANGE PROTEIN | | |
| | | 1) (AE 1). | | |
| ALTSNQEYLDL | FGR1_HUMAN | BASIC FIBROBLAST | A759 | Y766/ |
| SM | | GROWTH FACTOR | M771 | FGFR1 |
| | | RECEPTOR 1 | | |
| | | (BFGF-R) (EC | | |
| | | 2.7.1.112) (FMS-LIKE | | |
| | | TYROSINE KINASE- | | |
| | | 2) (C-FGR). | | |
| QNLNEDVSQEE | PRP5_HUMAN | BASIC PROLINE- | Q1 P13 | S8 |
| SP | | RICH PEPTIDE IB-1. | | |
| EYEDENLYEGL | C79A_HUMAN | B-CELL ANTIGEN | E181 | Y182/C- |
| NL | | RECEPTOR | L193 | jun; Y188/ |
| | | COMPLEX | | Lyn |
| | | ASSOCIATED | | |
| | | PROTEIN ALPHA- | | |
| | | CHAIN (IG-ALPHA) | | |
| | | (MB-1 MEMBRANE | | |
| | | GLYCOPROTEIN) | | |
| | | (SURFACE-IGM- | | |
| | | ASSOCIATED | | |
| | | PROTEIN) | | |
| | | (MEMBRANE- | | |
| | | BOUND | | |
| | | IMMUNOGLOBULIN | | |
| | | ASSOCIATED | | |
| | | PROTEIN (CD79A). | | |
| LQNLAKASPVY | TRKB_HUMAN | BDNF/NT-3 GROWTH | L807 | Y817 |
| LD | | FACTORS RECEPTOR | D819 | (autophosphorylation) |
| | | (EC 2.7.1.112) (TRKB | | |
| | | TYROSINE KINASE) | | |
| | | (GP145-TRKB) (TRK- | | |
| | | B). | | |
| RDVYSTDYYRV | TRKB_HUMAN | BDNF/NT-3 GROWTH | R699 | Y702; |
| GG | | FACTORS RECEPTOR | G711 | Y706; Y707 |
| | | (EC 2.7.1.112) (TRKB | | (alle |
| | | TYROSINE KINASE) | | autophosphorylation |
| | | (GP145-TRKB) (TRK- | | |
| | | B). | | |
| PVIENPQYFGITN | TRKB_HUMAN | BDNF/NT-3 GROWTH | P509 | Y516 |
| | | FACTORS RECEPTOR | N521 | (autophosphorylation) |
| | | (EC 2.7.1.112) (TRKB | | |
| | | TYROSINE KINASE) | | |
| | | (GP145-TRKB) (TRK- | | |
| | | B). | | |
| MEQKKRVTMIL | ADDB_HUMAN | BETA ADDUCIN | M48 | T55/PKA |
| QS | | (ERYTHROCYTE | S60 | |
| | | ADDUCIN BETA | | |
| | | SUBUNIT). | | |
| GSPSKSPSKKKKK | ADDB_HUMAN | BETA ADDUCIN | G696 | S703/PKC |
| | | (ERYTHROCYTE | K708 | |
| | | ADDUCIN BETA | | |
| | | SUBUNIT). | | |
| KKKFRTPSFLKKS | ADDB_HUMAN | BETA ADDUCIN | K706 | S713/PKC, |
| | | (ERYTHROCYTE | S718 | PKA |
| | | ADDUCIN BETA | | |
| | | SUBUNIT). | | |
| ALALARETIESLS | CASB_HUMAN | BETA CASEIN. | A11 S23 | T18 S21 |
| | | | | S23 |
| TIESLSSSEESIT | CASB_HUMAN | BETA CASEIN. | T18 T30 | T18; S21; |
| | | | | S23; S24; |
| | | | | S25 |
| LARETIESLSSSE | CASB_HUMAN | BETA CASEIN. | L14 E26 | T18; S21; |
| | | | | S23; S24; |
| | | | | S25 |
| IESLSSSEESITE | CASB_HUMAN | BETA CASEIN. | I19 E31 | S21; S23; |
| | | | | S24; S25 |
| DMKGDVKYADI | A002-G | BETA PLATELET- | D756 | Y763/ |
| ES | | DERIVED GROWTH | S768 | PDGFR1 |
| | | FACTOR RECEPTOR | | |
| SSNYMAPYDNY | A002-I | BETA PLATELET- | S768 | Y771/ |
| VP | | DERIVED GROWTH | P780 | PDGFR; |
| | | FACTOR RECEPTOR | | Y775/ |
| | | | | PDGFR; |
| | | | | Y778/ |
| | | | | PDGFR |
| VSSDGHEYIYV | A002-C | BETA PLATELET- | V572 | Y579/ |
| DP | | DERIVED GROWTH | P584 | PDGFR; |
| | | FACTOR RECEPTOR | | Y581/ |
| | | | | PDGFR |
| LDTSSVLYTAV | A002-K | BETA PLATELET- | L1002 | Y1009/ |
| QP | | DERIVED GROWTH | P1014 | PDGFR |
| | | FACTOR RECEPTOR | | |
| RPPSAELYSNALP | A002-E | BETA PLATELET- | R709 | Y716/ |
| | | DERIVED GROWTH | P721 | PDGFR |
| | | FACTOR RECEPTOR | | |
| ADIESSNYMAP | A002-H | BETA PLATELET- | A764 | Y771/ |
| YD | | DERIVED GROWTH | D776 | PDGFR; |
| | | FACTOR RECEPTOR | | Y775/ |
| | | | | PDGFR |
| TGESDGGYMD | A002-F | BETA PLATELET- | T733 | Y740/ |
| MSK | | DERIVED GROWTH | K745 | PDGFR |
| | | FACTOR RECEPTOR | | |
| YMAPYDNYVPS | A002-J | BETA PLATELET- | Y771 | Y771/ |
| AP | | DERIVED GROWTH | P783 | PDGFR; |
| | | FACTOR RECEPTOR | | Y775/ |
| | | | | PDGFR; |
| | | | | Y778/ |
| | | | | PDGFR |
| DIMRDSNYISKGS | PGDR_HUMAN | BETA PLATELET- | D850 | Y857/ |
| | | DERIVED GROWTH | S862 | PDGFR |
| | | FACTOR RECEPTOR | | |
| | | (EC 2.7.1.112) (PDGF- | | |
| | | R-BETA) (CD140B | | |
| | | ANTIGEN). | | |
| SKDESVDYVPM | PGDR_HUMAN | BETA PLATELET- | S744 | Y751/ |
| LD | | DERIVED GROWTH | D756 | PDGFR |
| | | FACTOR RECEPTOR | | |
| | | (EC 2.7.1.112) (PDGF- | | |
| | | R-BETA) (CD140B | | |
| | | ANTIGEN). | | |
| RAGKRRPSRLV | B1AR_HUMAN | BETA-1 | R305 | S312/PKA |
| AL | | ADRENERGIC | L317 | |
| | | RECEPTOR. | | |
| HGDRPRASGCL | B1AR_HUMAN | BETA-1 | H405 | S412/PKA |
| AR | | ADRENERGIC | R417 | |
| | | RECEPTOR. | | |
| DSQGRNCSTND | B204-C | BETA-2 | D400 | S401/ |
| SL | | ADRENERGIC | L411 | GRK2, |
| | | RECEPTOR | | GRK5; |
| | | | | S407/ |
| | | | | GRK2, |
| | | | | GRK5; |
| | | | | S411/ |
| | | | | GRK5 |
| VPSDNIDSQGRNC | B204-B | BETA-2 | V394 | S396/ |
| | | ADRENERGIC | C406 | GRK2, |
| | | RECEPTOR | | GRK5; |
| | | | | S401/ |
| | | | | GRK2, |
| | | | | GRK5 |
| DFVGHQGTVPS | B204-F | BETA-2 | D386 | T393/ |
| DN | | ADRENERGIC | N398 | GRK5; |
| | | RECEPTOR | | S396/ |
| | | | | GRK2, |
| | | | | GRK5 |
| SQGRNCSTNDS | B204-D | BETA-2 | S401 | S401/ |
| LL | | ADRENERGIC | L413 | GRK2, |
| | | RECEPTOR | | GRK5; |
| | | | | S407/ |
| | | | | GRK2, |
| | | | | GRK5; |
| | | | | S411/ |
| | | | | GRK5 |
| LCEDLPGTEDFVG | B204-E | BETA-2 | L377 | T384/ |
| | | ADRENERGIC | G389 | GRK2, |
| | | RECEPTOR | | GRK5 |
| GHQGTVPSDNI | B204-A | BETA-2 | G389 | T393/ |
| DS | | ADRENERGIC | S401 | GRK5; |
| | | RECEPTOR | | S396/ |
| | | | | GRK2, |
| | | | | GRK5; |
| | | | | S401/ |
| | | | | GRK2, |
| | | | | GRK5 |
| KAYGNGYSSNG | B2AR_HUMAN | BETA-2 | K348 | Y350/ |
| NT | | ADRENERGIC | T360 | TYR- |
| | | RECEPTOR. | | kinase; |
| | | | | Y354/ |
| | | | | TYR- |
| | | | | kinase; |
| | | | | S355/ |
| | | | | BARK; |
| | | | | S356/ |
| | | | | BARK |
| ELLCLRRSSLKAY | B2AR_HUMAN | BETA-2 | E338 | S345/PKC, |
| | | ADRENERGIC | Y350 | PKA, |
| | | RECEPTOR. | | betaARK; |
| | | | | S346/PKC, |
| | | | | PKA, |
| | | | | betaARK |
| APNVHINTIEPVN | KRAB_HUMAN | B-RAF PROTO- | A365 | T 372 |
| | | ONCOGENE | N377 | (autophosphorylation) |
| | | SERINE/THREONINE- | | |
| | | PROTEIN KINASE | | |
| | | (EC 2.7.1.—) (P94). | | |
| DPGSVLSTACG | KCC1_HUMAN | CALCIUM/CALMODULIN- | D170 | T177/ |
| TP | | DEPENDENT | P182 | calcium- |
| | | PROTEIN KINASE | | calmodulin |
| | | TYPE I (EC 2.7.1.123) | | PKI |
| | | (CAM KINASE I). | | |
| APTKRNSSPPPSP | B089-A | CALCIUM- | A1171 | S1178/ |
| | | TRANSPORTING | P1183 | PKA |
| | | ATPASE PLASMA | | |
| | | MEMBREANE | | |
| | | (CALCIUM PUMP) | | |
| LDRDGSRSLDA | CAYP_HUMAN | CALCYPHOSINE. | L33 E45 | S40/PKA |
| DE | | | | |
| INEWLTKTPDG | CALD_HUMAN | CALDESMON (CDM). | I746 | T753/ |
| NK | | | K758 | CDC2 |
| | | | | Kinase |
| EKGNVFSSPTA | CALD_HUMAN | CALDESMON (CDM). | E717 | S724/ |
| AG | | | G729 | CDC2 |
| | | | | Kinase |
| SSPTAAGTPNKET | CALD_HUMAN | CALDESMON (CDM). | S723 | S724/ |
| | | | T735 | CDC2 |
| | | | | Kinase; |
| | | | | T730/ |
| | | | | CDC2 |
| | | | | Kinase |
| KTPDGNKSPAP | CALD_HUMAN | CALDESMON (CDM). | K752 | S753/ |
| KP | | | P764 | CDC2 |
| | | | | Kinase; |
| | | | | S759/ |
| | | | | CDC2 |
| | | | | Kinase |
| KDGNGYISAAE | B227-A | CALMODULIN | K94 | Y99/INSR; |
| LR | | | R106 | S101/CK2 |
| FDKDGNGYISA | B227-C | CALMODULIN | F92 | Y99/INSR; |
| AE | | | E104 | S101/CK2 |
| MARKMKDTDS | B227-B | CALMODULIN | M72 | T79/CK2 |
| EEE | | | E84 | |
| TPQTQSTSGRRRR | B116-B | cAMP RESPONSE | T333 | S340/PKC |
| | | ELEMENT-BINDING | R345 | |
| | | PROTEIN CRE-BP1 | | |
| FGPARNDSVIVAD | B116-A | cAMP RESPONSE | F55 D67 | S62/PKA |
| | | ELEMENT-BINDING | | |
| | | PROTEIN CRE-BP1 | | |
| ERNRAAASRCR | B116-C | cAMP RESPONSE | E360 | S367/PKC |
| QK | | ELEMENT-BINDING | K372 | |
| | | PROTEIN CRE-BP1 | | |
| SRFNRRVSVCA | KAP2_HUMAN | CAMP-DEPENDENT | S91 | S98 |
| ET | | PROTEIN KINASE | T103 | |
| | | TYPE II-ALPHA | | |
| | | REGULATORY | | |
| | | CHAIN. | | |
| NRFTRRASVCA | KAP3_HUMAN | CAMP-DEPENDENT | N106 | S113/ |
| EA | | PROTEIN KINASE | A118 | autophosphorylation |
| | | TYPE II-BETA | | |
| | | REGULATORY | | |
| | | CHAIN. | | |
| EEEDIRVSITEKC | KAPB_HUMAN | CAMP-DEPENDENT | E331 | S338 |
| | | PROTEIN KINASE, | C343 | |
| | | BETA-CATALYTIC | | |
| | | SUBUNIT (EC | | |
| | | 2.7.1.37) (PKA C- | | |
| | | BETA). | | |
| RVKGRTWTLCG | KAPG_HUMAN | CAMP-DEPENDENT | R190 | T197/ |
| TP | | PROTEIN KINASE, | P202 | autophosphorylation |
| | | GAMMA-CATALYTIC | | |
| | | SUBUNIT (EC | | |
| | | 2.7.1.37) (PKA C- | | |
| | | GAMMA). | | |
| QKRREILSRRPSY | B015-A | cAMP-RESPONSE | Q122 | S133/ |
| | | ELEMENT BINDING | Y134 | PKA, |
| | | PROTEIN (CREB) | | MAPK, |
| | | | | CaMK, |
| | | | | RSK2 |
| EILSRRPSYRKIL | B015-B | cAMP-RESPONSE | E126 | S133/ |
| | | ELEMENT BINDING | L138 | PKA, |
| | | PROTEIN (CREB) | | MAPK, |
| | | | | CaMK, |
| | | | | RSK2 |
| RSAIRRASTIEMP | PPLA_HUMAN | CARDIAC | R9 P21 | S16/PKA; |
| | | PHOSPHOLAMBAN | | T17/ |
| | | (PLB). | | CAMK |
| MSSSEEVSWIS | KC2B_HUMAN | CASEIN KINASE II | M1 F13 | S2/ |
| WF | | BETA CHAIN (CK II) | | autophosphorylation |
| | | (EC 2.7.1.37) | | |
| | | (PHOSVITIN). | | |
| PLGPLAGSPVIAA | B197-B | CASEIN KINASE II, | P363 | S370/ |
| | | ALPHA CHAIN (CKII) | A375 | p34cdc2 |
| | | (EC 2.7.1.37) | | |
| ISSVPTPSPLGPL | B197-A | CASEIN KINASE II, | I355 | T360/ |
| | | ALPHA CHAIN (CKII) | L367 | p34cdc2; |
| | | (EC 2.7.1.37) | | S362/ |
| | | | | p34cdc2 |
| SSMPGGSTPVSSA | B197-C | CASEIN KINASE II, | S337 | T344/ |
| | | ALPHA CHAIN (CKII) | A349 | p34cdc2 |
| | | (EC 2.7.1.37) | | |
| SGISSVPTPSPLG | B197-D | CASEIN KINASE II, | S353 | T360/ |
| | | ALPHA CHAIN (CKII) | G365 | p34cdc2; |
| | | (EC 2.7.1.37) | | S362/ |
| | | | | p34cdc2 |
| HQRRKYRSNKG | TNR7_HUMAN | CD27L RECEPTOR (T- | H212 | S219 |
| ES | | CELL ACTIVATION | S224 | |
| | | ANTIGEN CD27) | | |
| | | (T14). | | |
| YDPAKRISGKM | B196-A | CELL DIVISION | Y270 | |
| AL | | CONTROL PROTEIN 2 | L282 | |
| | | HOMOLOG (P34 | | |
| | | PROTEIN KINASE) | | |
| GVPVRTYTHEV | CDK2_HUMAN | CELL DIVISION | G153 | T160/CAK |
| VT | | PROTEIN KINASE 2 | T165 | |
| | | (EC 2.7.1.—) (P33 | | |
| | | PROTEIN KINASE). | | |
| EKIGEGTYGVV | CDK2_HUMAN | CELL DIVISION | E8 K20 | T14; Y15 |
| YK | | PROTEIN KINASE 2 | | |
| | | (EC 2.7.1.—) (P33 | | |
| | | PROTEIN KINASE). | | |
| YSYQMALTPVV | CDK4_HUMAN | CELL DIVISION | Y165 | T172/CAK |
| VT | | PROTEIN KINASE 4 | T177 | |
| | | (EC 2.7.1.—) (CYCLIN- | | |
| | | DEPENDENT KINASE | | |
| | | 4) (PSK-J3). | | |
| GIPVRCYSAEVVT | CDK5_HUMAN | CELL DIVISION | G152 | S159/CK1 |
| | | PROTEIN KINASE 5 | T164 | |
| | | (EC 2.7.1.—) (TAU | | |
| | | PROTEIN KINASE II | | |
| | | CATALYTIC | | |
| | | SUBUNIT) (TPKII | | |
| | | CATALYTIC | | |
| | | SUBUNIT) | | |
| | | (SERINE/THREONINE- | | |
| | | PROTEIN KINASE | | |
| | | PSSALRE). | | |
| LEKIGEGTYGTVF | CDK5_HUMAN | CELL DIVISION | L7 F19 | T14; Y15/ |
| | | PROTEIN KINASE 5 | | c-Abl kinase |
| | | (EC 2.7.1.—) (TAU | | |
| | | PROTEIN KINASE II | | |
| | | CATALYTIC | | |
| | | SUBUNIT) (TPKII | | |
| | | CATALYTIC | | |
| | | SUBUNIT) | | |
| | | (SERINE/THREONINE- | | |
| | | PROTEIN KINASE | | |
| | | PSSALRE). | | |
| GSPNRAYTHQV | CDK7_HUMAN | CELL DIVISION | G163 | S164/ |
| VT | | PROTEIN KINASE 7 | T175 | CDC2, |
| | | (EC 2.7.1.—) (CDK- | | CDK2; |
| | | ACTIVATING | | T170/ |
| | | KINASE) (CAK) (39 KDA | | CDC2, |
| | | PROTEIN | | CDK2 |
| | | KINASE) (P39 | | |
| | | MO15) (STK1) | | |
| | | (CAK1). | | |
| GLAKSFGSPNR | CDK7_HUMAN | CELL DIVISION | G157 | S164/ |
| AY | | PROTEIN KINASE 7 | Y169 | CDC2, |
| | | (EC 2.7.1.—) (CDK- | | CDK2 |
| | | ACTIVATING | | |
| | | KINASE) (CAK) (39 KDA | | |
| | | PROTEIN | | |
| | | KINASE) (P39 | | |
| | | MO15) (STK1) | | |
| | | (CAK1). | | |
| PSVEPPLSQETFS | P53_HUMAN | CELLULAR TUMOR | P8 S20 | S15/ATR, |
| | | ANTIGEN P53 | | PRPK; T18/ |
| | | (PHOSPHOPROTEIN | | CK1, |
| | | P53). | | ATR, |
| | | | | VRK1; S20/ |
| | | | | PLK3, ATR |
| NVLSPLPSQAM | P53_HUMAN | CELLULAR TUMOR | N30 | S33/GSK3 |
| DD | | ANTIGEN P53 | D42 | beta kinase, |
| | | (PHOSPHOPROTEIN | | CAK; S37/ |
| | | P53). | | ATR |
| PPLSQETFSDLWK | P53_HUMAN | CELLULAR TUMOR | P12 K24 | S15/ATR, |
| | | ANTIGEN P53 | | PRPK; T18/ |
| | | (TUMOR | | CK1, |
| | | SUPPRESSOR P53) | | ATR, |
| | | (PHOSPHOPROTEIP53) | | VRK1; S20/ |
| | | | | PLK3, |
| | | | | ATR |
| HHKLVLPSNTP | CENC_HUMAN | CENTROMERE | H725 | S732 |
| NV | | PROTEIN C (CENP-C) | N737 | |
| | | (CENTROMERE | | |
| | | AUTOANTIGEN C). | | |
| CADVPLLTPSSKE | ETS1_HUMAN | C-ETS-1 PROTEIN | C31 E43 | T38/ |
| | | (P54) (C-ETS-1A AND | | MAPK, |
| | | C-ETS-1B). | | HGF/SF |
| THIGPRTTRAQGI | KGPA_HUMAN | CGMP-DEPENDENT | T51 I63 | T58/PKC |
| | | PROTEIN KINASE 1, | | |
| | | ALPHA ISOZYME (EC | | |
| | | 2.7.1.37) (CGK 1 | | |
| | | ALPHA) (CGKI- | | |
| | | ALPHA). | | |
| GTPTRKISASEFD | CN5A_HUMAN | CGMP-SPECIFIC 3′,5′- | G95 | S102/ |
| | | CYCLIC | D107 | cGMP |
| | | PHOSPHODIESTERASE | | dependent |
| | | (EC | | protein |
| | | 3.1.4.17) (CGB-PDE) | | kinase |
| | | (CGMP-BINDING | | |
| | | CGMP-SPECIFIC | | |
| | | PHOSPHODIESTERASE). | | |
| YRDVRFESIRLPG | B091-A | CHORIOGONATROPIN | Y79 | S86/PKA |
| | | BETA CHAIN | G91 | |
| QCALCRRSTTD | B091-B | CHORIOGONATROPIN | Q109 | S116/ |
| CG | | BETA CHAIN | G121 | PKA; T117/ |
| | | | | PKA |
| QRRSARLSAKP | B070-A | CHROMOSOMAL | Q21 P33 | S28/PKG |
| AP | | HIGH MOBILITY | | |
| | | GROUP PROTEIN 17 | | |
| | | (HMG-17) | | |
| SQITSQVTGQIGW | AP50_HUMAN | CLATHRIN COAT | S149 | T156 |
| | | ASSEMBLY PROTEIN | W161 | |
| | | AP50 (CLATHRIN | | |
| | | COAT ASSOCIATED | | |
| | | PROTEIN AP50) | | |
| | | (PLASMA | | |
| | | MEMBRANE | | |
| | | ADAPTOR AP-2 50 KDA | | |
| | | PROTEIN) (HA2 | | |
| | | 50 KDA SUBUNIT) | | |
| | | (CLATHRIN | | |
| | | ASSEMBLY PROTEIN | | |
| | | COMPLEX 2 MEDIUM | | |
| | | CHAIN) (AP 2 MU 2 | | |
| | | CHAIN) (KIAA0109). | | |
| DFGFFSSSESGAP | CLCB_HUMAN | CLATHRIN LIGHT | D4 P16 | S11/CK2; |
| | | CHAIN B (BRAIN | | S13/CK2 |
| | | AND LYMPHOCYTE | | |
| | | LCB). | | |
| NLNGREFSGRA | CST2_HUMAN | CLEAVAGE | N76 | S83/PKC |
| LR | | STIMULATION | R88 | (potential) |
| | | FACTOR, 64 KDA | | |
| | | SUBUNIT (CSTF | | |
| | | 64 KDA SUBUNIT) | | |
| | | (CF-1 64 KDA | | |
| | | SUBUNIT). | | |
| HHVPGHESRGP | CST2_HUMAN | CLEAVAGE | H357 | S364/PKG |
| PP | | STIMULATION | P369 | (potential) |
| | | FACTOR, 64 KDA | | |
| | | SUBUNIT (CSTF | | |
| | | 64 KDA SUBUNIT) | | |
| | | (CF-1 64 KDA | | |
| | | SUBUNIT). | | |
| GAVVPQGSRQV | CST2_HUMAN | CLEAVAGE | G491 | S498/PKG |
| PV | | STIMULATION | V503 | (potential) |
| | | FACTOR, 64 KDA | | |
| | | SUBUNIT (CSTF | | |
| | | 64 KDA SUBUNIT) | | |
| | | (CF-1 64 KDA | | |
| | | SUBUNIT). | | |
| SCKDDINSYEC | FA9_HUMAN | COAGULATION | S107 | S114 |
| WC | | FACTOR IX (EC | C119 | |
| | | 3.4.21.22) | | |
| | | (CHRISTMAS | | |
| | | FACTOR). | | |
| DMKVRKSSTPE | COF1_HUMAN | COFILIN, NON- | D17 | S24 |
| EV | | MUSCLE ISOFORM. | V29 | |
| LKGKRGDSGSP | CA34_HUMAN | COLLAGEN ALPHA | L1428 | S1435; |
| AT | | 3(IV) CHAIN. | T1440 | S1437 |
| TEASGYISSLEYP | C1R_HUMAN | COMPLEMENT C1R | T199 | S206/CK2 |
| | | COMPONENT (EC | P211 | |
| | | 3.4.21.41). | | |
| VSQREAEYEPETV | A008-B | CORTACTIN, Src | V470 | Y477/v-Src |
| | | SUBSTRATE P80/85 | V482 | |
| | | PROTEINS | | |
| YQAEENTYDEY | A008-D | CORTACTIN, Src | Y492 | Y499/v- |
| EN | | SUBSTRATE P80/85 | N504 | Src |
| | | PROTEINS | | |
| EYEPETVYEVA | A008-C | CORTACTIN, SRC | E476 | Y477/v- |
| GA | | SUBSTRATE P80/85 | A488 | Src; Y483/ |
| | | PROTEINS | | v-Src; |
| KTPSSPVYQDA | A008-A | CORTACTIN, SRC | K423 | Y430/v- |
| VS | | SUBSTRATE P80/85 | S435 | Src |
| | | PROTEINS | | |
| KMQLRRPSDQE | REL_HUMAN | C-REL PROTO- | K260 | S267/PKA |
| VS | | ONCOGENE PROTEIN | S272 | |
| | | (C-REL PROTEIN). | | |
| SVIVADQTPTPTR | ATF2_HUMAN | CYCLIC-AMP- | S44 R56 | T51/ |
| | | DEPENDENT | | MAPK14; |
| | | TRANSCRIPTION | | T53/ |
| | | FACTOR ATF-2 | | MAPK14 |
| | | (ACTIVATING | | |
| | | TRANSCRIPTION | | |
| | | FACTOR 2) (CAMP | | |
| | | RESPONSE ELEMENT | | |
| | | BINDING PROTEIN | | |
| | | CRE-BP1) (HB16). | | |
| YEDDDYVSKKS | CYCH_HUMAN | CYCLIN H (MO15- | Y297 | S304/ |
| KH | | ASSOCIATED | H309 | CDK8 |
| | | PROTEIN) (P37) (P34). | | |
| VPWEDRMSLVN | B311-C | CYSTATIN S | V125 | S132; S136 |
| SR | | (SALIVARY ACIDIC | R137 | |
| | | PROTEIN-1) | | |
| AGALASSSKEE | B311-A | CYSTATIN S | A16 | S21; S23 |
| NR | | (SALIVARY ACIDIC | R28 | |
| | | PROTEIN-1) | | |
| DRMSLVNSRCQ | B311-D | CYSTATIN S | D129 | S132; S136 |
| EA | | (SALIVARY ACIDIC | A141 | |
| | | PROTEIN-1) | | |
| LQKKQLCSFEIYE | B311-B | CYSTATIN S | L112 | S119 |
| | | (SALIVARY ACIDIC | E124 | |
| | | PROTEIN-1) | | |
| EAILPRISVISTG | CFTR_HUMAN | CYSTIC FIBROSIS | E746 | S753/PKA |
| | | TRANSMEMBRANE | G758 | |
| | | CONDUCTANCE | | |
| | | REGULATOR (CFTR) | | |
| | | (CAMP-DEPENDENT | | |
| | | CHLORIDE | | |
| | | CHANNEL). | | |
| WTETKKQSFKQ | CFTR_HUMAN | CYSTIC FIBROSIS | W679 | S686/PKC |
| TG | | TRANSMEMBRANE | G691 | |
| | | CONDUCTANCE | | |
| | | REGULATOR (CFTR) | | |
| | | (CAMP-DEPENDENT | | |
| | | CHLORIDE | | |
| | | CHANNEL). | | |
| IHRKTTASTRKVS | CFTR_HUMAN | CYSTIC FIBROSIS | I783 | S790/PKC; |
| | | TRANSMEMBRANE | S795 | S795/ |
| | | CONDUCTANCE | | PKA, PKG |
| | | REGULATOR (CFTR) | | |
| | | (CAMP-DEPENDENT | | |
| | | CHLORIDE | | |
| | | CHANNEL). | | |
| TASTRKVSLAP | CFTR_HUMAN | CYSTIC FIBROSIS | T788 | S790/PKC; |
| QA | | TRANSMEMBRANE | A800 | S795/ |
| | | CONDUCTANCE | | PKA, PKG |
| | | REGULATOR (CFTR) | | |
| | | (CAMP-DEPENDENT | | |
| | | CHLORIDE | | |
| | | CHANNEL). | | |
| INSIRKFSIVQKT | CFTR_HUMAN | CYSTIC FIBROSIS | I705 | S712/PKA |
| | | TRANSMEMBRANE | T717 | |
| | | CONDUCTANCE | | |
| | | REGULATOR (CFTR) | | |
| | | (CAMP-DEPENDENT | | |
| | | CHLORIDE | | |
| | | CHANNEL). | | |
| LQARRRQSVLN | CFTR_HUMAN | CYSTIC FIBROSIS | L761 | S768/ |
| LM | | TRANSMEMBRANE | M773 | PKA; PKG |
| | | CONDUCTANCE | | |
| | | REGULATOR (CFTR) | | |
| | | (CAMP-DEPENDENT | | |
| | | CHLORIDE | | |
| | | CHANNEL). | | |
| FGEKRKNSILNPI | CFTR_HUMAN | CYSTIC FIBROSIS | F693 | S700/ |
| | | TRANSMEMBRANE | I705 | PKA, PKG |
| | | CONDUCTANCE | | |
| | | REGULATOR (CFTR) | | |
| | | (CAMP-DEPENDENT | | |
| | | CHLORIDE | | |
| | | CHANNEL). | | |
| EPLERRLSLVPDS | CFTR_HUMAN | CYSTIC FIBROSIS | E730 | S737/ |
| | | TRANSMEMBRANE | S742 | PKA; PKG |
| | | CONDUCTANCE | | |
| | | REGULATOR (CFTR) | | |
| | | (CAMP-DEPENDENT | | |
| | | CHLORIDE | | |
| | | CHANNEL). | | |
| WKVLRRFSVTT | CPB6_HUMAN | CYTOCHROME P450 | W121 | S128/PKA |
| MR | | 2B6 (EC 1.14.14.1) | R133 | |
| | | (CYPIIB6) (P450 | | |
| | | IIB1). | | |
| LNTSYPLSPLSDF | PA2Y_HUMAN | CYTOSOLIC | L498 | S505/ |
| | | PHOSPHOLIPASE A2 | F510 | MAPK |
| | | (CPLA2) [INCLUDES: | | |
| | | PHOSPHOLIPASE A2 | | |
| | | (EC 3.1.1.4) (CPLA2) | | |
| | | (PHOSPHATIDYLCHOLINE | | |
| | | 2- | | |
| | | ACYLHYDROLASE); | | |
| | | LYSOPHOSPHOLIPASE | | |
| | | (EC 3.1.1.5)]. | | |
| SEETPAISPSKRA | B314-A | DEOXYURIDINE 5′- | S4 A16 | S11 |
| | | TRIPHOSPHATE | | |
| | | NUCLEOTIDOHYDROLASE | | |
| | | (EC 3.6.1.23) | | |
| YNYEGRGSVAG | DSC2_HUMAN | DESMOCOLLIN | Y857 | S864 |
| SV | | 2A/2B | V869 | |
| | | (DESMOSOMAL | | |
| | | GLYCOPROTEIN II | | |
| | | AND III) | | |
| | | (DESMOCOLLIN-3). | | |
| RSGSRRGSFDA | DESP_HUMAN | DESMOPLAKIN I | R2842 | S2849/ |
| TG | | AND II (DPI AND | G2854 | PKA |
| | | DPII) (FRAGMENT). | | |
| VCDCKRNSDV | CIC2_HUMAN | DIHYDROPYRIDINE- | V826 | S833/PKA |
| MDC | | SENSITIVE L-TYPE, | C838 | |
| | | CALCIUM CHANNEL | | |
| | | ALPHA-2/DELTA | | |
| | | SUBUNITS. | | |
| LEDIKRLTPRFTL | CIC2_HUMAN | DIHYDROPYRIDINE- | L494 | T501/PKA |
| | | SENSITIVE L-TYPE, | L506 | |
| | | CALCIUM CHANNEL | | |
| | | ALPHA-2/DELTA | | |
| | | SUBUNITS. | | |
| DDSIISSLDVTDI | Q9UP94 | DNA REPAIR | D254 | DNA-PK |
| | | PROTEIN XRCC4 (X- | I266 | |
| | | RAY REPAIR | | |
| | | COMPLEMENTING | | |
| | | DEFECTIVE REPAIIN | | |
| | | CHINESE HAMSTER | | |
| | | CELLS 4) | | |
| RPNPCAYTPPSLK | IPPD_HUMAN | DOPAMINE- AND | R68 | T75/Cdk5 |
| | | CAMP-REGULATED | K80 | |
| | | NEURONAL | | |
| | | PHOSPHOPROTEIN | | |
| | | (DARPP-32). | | |
| LLADLTRSLSDNI | DCX_HUMAN | DOUBLECORTIN | L125 | S132/CK2 |
| | | (LISSENCEPHALIN- | I137 | |
| | | X) (LIS-X) | | |
| | | (DOUBLIN). | | |
| STPKSKQSPISTP | DCX_HUMAN | DOUBLECORTIN | S362 | T363/PKC |
| | | (LISSENCEPHALIN- | P374 | oder |
| | | X) (LIS-X) | | MAPK; |
| | | (DOUBLIN). | | S369/ |
| | | | | MAPK; |
| | | | | T373/ |
| | | | | MAPK |
| YIYTIDGSRKIGS | DCX_HUMAN | DOUBLECORTIN | Y145 | S152/PKC; |
| | | (LISSENCEPHALIN- | S157 | S157/CK2 |
| | | X) (LIS-X) | | |
| | | (DOUBLIN). | | |
| KDLYLPLSLDDSD | DCX_HUMAN | DOUBLECORTIN | K384 | S391/CK2 |
| | | (LISSENCEPHALIN- | D396 | |
| | | X) (LIS-X) | | |
| | | (DOUBLIN). | | |
| HFDERDKTSRN | DCX_HUMAN | DOUBLECORTIN | H49 | T56/PKC |
| MR | | (LISSENCEPHALIN- | R61 | |
| | | X) (LIS-X) | | |
| | | (DOUBLIN). | | |
| GPMRRSKSPAD | DCX_HUMAN | DOUBLECORTIN | G341 | S348/CK2 |
| SA | | (LISSENCEPHALIN- | A353 | |
| | | X) (LIS-X) | | |
| | | (DOUBLIN). | | |
| TSSSQLSTPKSKQ | DCX_HUMAN | DOUBLECORTIN | T356 | T363/PKC |
| | | (LISSENCEPHALIN- | Q368 | oder MAPK |
| | | X) (LIS-X) | | |
| | | (DOUBLIN). | | |
| SKQSPISTPTSPG | DCX_HUMAN | DOUBLECORTIN | S366 | S369/ |
| | | (LISSENCEPHALIN- | G378 | MAPK; |
| | | X) (LIS-X) | | T373/ |
| | | (DOUBLIN). | | MAPK; |
| | | | | S376/ |
| | | | | MAPK |
| RYAQDDFSLDE | DCX_HUMAN | DOUBLECORTIN | R300 | S307/CK2 |
| NE | | (LISSENCEPHALIN- | E312 | |
| | | X) (LIS-X) | | |
| | | (DOUBLIN). | | |
| SPISTPTSPGSLR | DCX_HUMAN | DOUBLECORTIN | S369 | S369/ |
| | | (LISSENCEPHALIN- | R381 | MAPK; |
| | | X) (LIS-X) | | T373/ |
| | | (DOUBLIN). | | MAPK; |
| | | | | S376/ |
| | | | | MAPK; |
| | | | | S379/PKC |
| STPTSPGSLRKHK | DCX_HUMAN | DOUBLECORTIN | S372 | T373/ |
| | | (LISSENCEPHALIN- | K384 | MAPK; |
| | | X) (LIS-X) | | S376/ |
| | | (DOUBLIN). | | MAPK; |
| | | | | S379/PKC |
| DLYLPLSLDDSDS | DCX_HUMAN | DOUBLECORTIN | D385 | S391/CK2; |
| | | (LISSENCEPHALIN- | S397 | S397/CK2 |
| | | X) (LIS-X) | | |
| | | (DOUBLIN). | | |
| GIVYAVSSDRFRS | DCX_HUMAN | DOUBLECORTIN | G109 | Y112/ |
| | | (LISSENCEPHALIN- | S121 | ABL; S116/ |
| | | X) (LIS-X) | | PKC |
| | | (DOUBLIN). | | |
| EQQLFYISQPGSS | MR11_HUMAN | DOUBLE-STRAND | E257 | |
| | | BREAK REPAIR | S269 | |
| | | PROTEIN MRE11A | | |
| | | (MRE11 HOMOLOG | | |
| | | 1). | | |
| SGQLIDSMANSFV | MPK1_HUMAN; | DUAL SPECIFICITY | S211 | S217/ |
| | MPK2_HUMAN | MITOGEN- | V223 | p74raf-1; |
| | | ACTIVATED | | S221/ |
| | | PROTEIN KINASE | | p74raf-1 |
| | | KINASE (EC 2.7.1.—) | | |
| | | (MAP KINASE | | |
| | | KINASE 1) (MAPKK | | |
| | | 1) (ERK | | |
| | | ACTIVATOR KINASE | | |
| | | 1(MAPK/ERK KINASE | | |
| | | 1) (MEK1) | | |
| IDSMANSFVGT | MPK1_HUMAN; | DUAL SPECIFICITY | I215 | S217/ |
| RS | MPK2_HUMAN | MITOGEN- | S227 | p74raf-1; |
| | | ACTIVATED | | S221/ |
| | | PROTEIN KINASE | | p74raf-1 |
| | | KINASE (EC 2.7.1.—) | | |
| | | (MAP KINASE | | |
| | | KINASE 1) (MAPKK | | |
| | | 1) (ERK | | |
| | | ACTIVATOR KINASE | | |
| | | 1(MAPK/ERK KINASE | | |
| | | 1) (MEK1) | | |
| LIDSMANSFVGTR | MPK1_HUMAN | DUAL SPECIFICITY | L214 | S217/ |
| | | MITOGEN- | R226 | p74raf-1; |
| | | ACTIVATED | | S221/ |
| | | PROTEIN KINASE | | p74raf-1 |
| | | KINASE 1 (EC 2.7.1.—) | | |
| | | (MAP KINASE | | |
| | | KINASE 1) (MAPKK | | |
| | | 1) | | |
| | | (ERK ACTIVATOR | | |
| | | KINASE 1) | | |
| | | (MAPK/ERK KINASE | | |
| | | 1) (MEK1). | | |
| VSGQLIDSMAN | MPK2_HUMAN | DUAL SPECIFICITY | V215 | S222/RAF; |
| SF | | MITOGEN- | F227 | S226/RAF |
| | | ACTIVATED | | |
| | | PROTEIN KINASE | | |
| | | KINASE 2 (EC 2.7.1.—) | | |
| | | (MAP KINASE | | |
| | | KINASE 2) (MAPKK | | |
| | | 2) | | |
| | | (ERK ACTIVATOR | | |
| | | KINASE 2) | | |
| | | (MAPK/ERK KINASE | | |
| | | 2) (MEK2). | | |
| LVNSIAKTYVG | MPK5_HUMAN | DUAL SPECIFICITY | L308 | S311/ |
| TN | | MITOGEN- | N320 | autophosphorylation; |
| | | ACTIVATED | | T315/ |
| | | PROTEIN KINASE | | autophosphorylation |
| | | KINASE 5 (EC 2.7.1.—) | | |
| | | (MAP KINASE | | |
| | | KINASE 5) (MAPKK | | |
| | | 5) (MAPK/ERK | | |
| | | KINASE 5). | | |
| VSTQLVNSIAKTY | MPK5_HUMAN | DUAL SPECIFICITY | V304 | S311/ |
| | | MITOGEN- | Y316 | autophosphorylation; |
| | | ACTIVATED | | T315/ |
| | | PROTEIN KINASE | | autophosphorylation |
| | | KINASE 5 (EC 2.7.1.—) | | |
| | | (MAP KINASE | | |
| | | KINASE 5) (MAPKK | | |
| | | 5) | | |
| | | (MAPK/ERK KINASE | | |
| | | 5). | | |
| ISGYLVDSVAKTI | MPK6_HUMAN | DUAL SPECIFICITY | I200 | S207/ |
| | | MITOGEN- | I212 | autophosphorylation?; |
| | | ACTIVATED | | T211/ |
| | | PROTEIN KINASE | | autophosphorylation? |
| | | KINASE 6 (EC 2.7.1.—) | | |
| | | (MAP KINASE | | |
| | | KINASE 6) (MAPKK | | |
| | | 6) (MAPK/ERK | | |
| | | KINASE 6) (SAPKK3). | | |
| LVDSVAKTIDA | MPK6_HUMAN | DUAL SPECIFICITY | L204 | S207/ |
| GC | | MITOGEN- | C216 | autophosphorylation?; |
| | | ACTIVATED | | T211/ |
| | | PROTEIN KINASE | | autophosphorylation? |
| | | KINASE 6 (EC 2.7.1.—) | | |
| | | (MAP KINASE | | |
| | | KINASE 6) (MAPKK | | |
| | | 6) | | |
| | | (MAPK/ERK KINASE | | |
| | | 6) (SAPKK3). | | |
| CQLGQRIYQYIQS | DYRA_HUMAN | DUAL-SPECIFICITY | C312 | Y319/ |
| | | TYROSINE- | S324 | autophosphorylation; |
| | | PHOSPHORYLATION | | Y321/ |
| | | REGULATED KINASE | | autophosphorylation |
| | | 1A (EC 2.7.1.—) | | |
| | | (PROTEIN KINASE | | |
| | | MINIBRAIN | | |
| | | HOMOLOG) (MNBH) | | |
| | | (HP86) (DUAL | | |
| | | SPECIFICITY | | |
| | | YAK1-RELATED | | |
| | | KINASE). | | |
| KHDTEMKYYIV | DYRA_HUMAN | DUAL-SPECIFICITY | K212 | Y219/ |
| HL | | TYROSINE- | L224 | autophosphorylation |
| | | PHOSPHORYLATION | | |
| | | REGULATED KINASE | | |
| | | 1A (EC 2.7.1.—) | | |
| | | (PROTEIN KINASE | | |
| | | MINIBRAIN | | |
| | | HOMOLOG) (MNBH) | | |
| | | (HP86) (DUAL | | |
| | | SPECIFICITY | | |
| | | YAK1-RELATED | | |
| | | KINASE). | | |
| CYEQLNDSSEEED | B228-A | E7 PROTEIN | C24 | S31/CK2; |
| | | | D36 | S32/CK2 |
| TFPPAPGSPEPPH | B257-A | EARLY E1A 32 KD | T82 H94 | S89/ |
| | | PROTEIN | | |
| AILRRPTSPVSRE | B257-B | EARLY E1A 32 kD | A212 | S219 |
| | | PROTEIN | E224 | |
| ECNSSTDSCDSGP | B257-C | EARLY E1A 32 kD | E224 | S231 |
| | | PROTEIN | P236 | |
| MNMLMERYRV | DNB2_ADE04 | EARLY E2A DNA- | M167 | Y174 |
| ESD | | BINDING PROTEIN. | D179 | |
| AVRDMRQTVA | B163-A | ELONGATION | A425 | T432/PKC |
| VGV | | FACTOR 1-ALPHA 1 | V437 | delta |
| | | (EF-1-ALPHA-1) | | |
| DDIDLFGSDDEEE | EF1B_HUMAN | ELONGATION | D98 | S105/CK2 |
| | | FACTOR 1-BETA (EF- | E110 | |
| | | 1-BETA). | | |
| ASARAGETRFT | B219-A | ELONGATION | A46 T58 | T56/CaM- |
| DT | | FACTOR 2 (EF-2) | | III; T58/ |
| | | | | CaM-III |
| RAGETRFTDTR | EF2_HUMAN | ELONGATION | R49 | T56/CaM- |
| KD | | FACTOR 2 (EF-2). | D61 | III; T58/ |
| | | | | CaM-III |
| GGTDEGIYDVP | EFS_HUMAN | EMBRYONAL FYN- | G246 | Y253/src |
| LL | | ASSOCIATED | L258 | |
| | | SUBSTRATE (HEFS). | | |
| ESIRMKRYILHFH | EPA1_HUMAN | EPHRIN TYPE-A | E923 | Y930/ |
| | | RECEPTOR 1 (EC | H935 | autophosphorylation |
| | | 2.7.1.112) (TYROSINE- | | |
| | | PROTEIN KINASE | | |
| | | RECEPTOR EPH). | | |
| LDDFDGTYETQ | EPA1_HUMAN | EPHRIN TYPE-A | L774 | Y781/ |
| GG | | RECEPTOR 1 (EC | G786 | autophosphorylation |
| | | 2.7.1.112) (TYROSINE- | | |
| | | PROTEIN KINASE | | |
| | | RECEPTOR EPH). | | |
| ESIKMQQYTEH | EPA2_HUMAN | EPHRIN TYPE-A | E914 | Y921/ |
| FM | | RECEPTOR 2 (EC | M926 | autophosphorylation |
| | | 2.7.1.112) (TYROSINE- | | |
| | | PROTEIN KINASE | | |
| | | RECEPTOR ECK) | | |
| | | (EPITHELIAL CELL | | |
| | | KINASE). | | |
| EDDPEATYTTS | EPA2_HUMAN | EPHRIN TYPE-A | E765 | Y772/ |
| GG | | RECEPTOR 2 (EC | G777 | autophosphorylation |
| | | 2.7.1.112) (TYROSINE- | | |
| | | PROTEIN KINASE | | |
| | | RECEPTOR ECK) | | |
| | | (EPITHELIAL CELL | | |
| | | KINASE). | | |
| QLKPLKTYVDP | EPA2_HUMAN | EPHRIN TYPE-A | Q581 | Y588/ |
| HT | | RECEPTOR 2 (EC | T593 | autophosphorylation |
| | | 2.7.1.112) (TYROSINE- | | |
| | | PROTEIN KINASE | | |
| | | RECEPTOR ECK) | | |
| | | (EPITHELIAL CELL | | |
| | | KINASE). | | |
| KLPGLRTYVDP | EPA3_HUMAN | EPHRIN TYPE-A | K589 | Y596/ |
| HT | | RECEPTOR 3 (EC | T601 | autophosphorylation |
| | | 2.7.1.112) (TYROSINE- | | |
| | | PROTEIN KINASE | | |
| | | RECEPTOR ETK1) | | |
| | | (HEK) (HEK4). | | |
| LNQGVRTYVDP | EPA4_HUMAN | EPHRIN TYPE-A | L589 | Y596/ |
| FT | | RECEPTOR 4 (EC | T601 | autophosphorylation |
| | | 2.7.1.112) (TYROSINE- | | |
| | | PROTEIN KINASE | | |
| | | RECEPTOR SEK) | | |
| | | (RECEPTOR | | |
| | | PROTEIN- | | |
| | | TYROSINE KINASE | | |
| | | HEK8). | | |
| EAIKMGRYTEIFM | EPA5_HUMAN | EPHRIN TYPE-A | E975 | Y982/ |
| | | RECEPTOR 5 (EC | M987 | autophosphorylation |
| | | 2.7.1.112) (TYROSINE- | | |
| | | PROTEIN KINASE | | |
| | | RECEPTOR EHK-1) | | |
| | | (EPH HOMOLOGY | | |
| | | KINASE-1) | | |
| | | (RECEPTOR | | |
| | | PROTEIN-TYROSINE | | |
| | | KINASE HEK7). | | |
| TYIDPETYEDPNR | EPA7_HUMAN | EPHRIN TYPE-A | T607 | Y608/ |
| | | RECEPTOR 7 (EC | R619 | autophosphorylation; |
| | | 2.7.1.112) (TYROSINE- | | Y614/ |
| | | PROTEIN KINASE | | autophosphorylation |
| | | RECEPTOR EHK-3) | | |
| | | (EPH HOMOLOGY | | |
| | | KINASE-3) | | |
| | | (RECEPTOR | | |
| | | PROTEIN-TYROSINE | | |
| | | KINASE HEK11). | | |
| DDTSDPTYTSSLG | EPB1_HUMAN | EPHRIN TYPE-B | D771 | Y778/ |
| | | RECEPTOR 1 (EC | G783 | autophosphorylation |
| | | 2.7.1.112) (TYROSINE- | | |
| | | PROTEIN KINASE | | |
| | | RECEPTOR EPH-2) | | |
| | | (NET) (HEK6) (ELK). | | |
| GSPGMKIYIDPFT | EPB1_HUMAN | EPHRIN TYPE-B | G587 | Y594/ |
| | | RECEPTOR 1 (EC | T599 | autophosphorylation |
| | | 2.7.1.112) (TYROSINE- | | |
| | | PROTEIN KINASE | | |
| | | RECEPTOR EPH-2) | | |
| | | (NET) (HEK6) (ELK). | | |
| SAIKMVQYRDS | EPB1_HUMAN | EPHRIN TYPE-B | S921 | Y928/ |
| FL | | RECEPTOR 1 (EC | L933 | autophosphorylation |
| | | 2.7.1.112) (TYROSINE- | | |
| | | PROTEIN KINASE | | |
| | | RECEPTOR EPH-2) | | |
| | | (NET) (HEK6) (ELK). | | |
| AIKMVQYRDSF | EPB1_HUMAN | EPHRIN TYPE-B | S922 | Y928/ |
| LT | | RECEPTOR 1 (EC | L934 | autophosphorylation |
| | | 2.7.1.112) (TYROSINE- | | |
| | | PROTEIN KINASE | | |
| | | RECEPTOR EPH-2) | | |
| | | (NET) (HEK6) (ELK). | | |
| DAIKMGRYKES | EPB3_HUMAN | EPHRIN TYPE-B | D935 | Y942/ |
| FV | | RECEPTOR 3 (EC | V947 | autophosphorylation |
| | | 2.7.1.112) (TYROSINE- | | |
| | | PROTEIN KINASE | | |
| | | RECEPTOR HEK-2). | | |
| IGHGTKVYIDPFT | EPB4_HUMAN | EPHRIN TYPE-B | I583 | Y590/ |
| | | RECEPTOR 4 (EC | T595 | autophosphorylation |
| | | 2.7.1.112) (TYROSINE- | | |
| | | PROTEIN KINASE | | |
| | | RECEPTOR HTK). | | |
| ISLDNPDYQQDFF | EGFR_HUMAN | EPIDERMAL | I1165 | Y1172/ |
| | | GROWTH FACTOR | F1177 | autophosphorylation |
| | | RECEPTOR (EC | | |
| | | 2.7.1.112). | | |
| TFLPVPEYINQSV | EGFR_HUMAN | EPIDERMAL | T1085 | Y1092/ |
| | | GROWTH FACTOR | V1097 | autophosphorylation |
| | | RECEPTOR (EC | | |
| | | 2.7.1.112). | | |
| GSVQNPVYHNQ | EGFR_HUMAN | EPIDERMAL | G1103 | Y1110/ |
| PL | | GROWTH FACTOR | L1115 | autophosphorylation |
| | | RECEPTOR (EC | | |
| | | 2.7.1.112). | | |
| RHIVRKRTLRRLL | EGFR_HUMAN | EPIDERMAL | R671 | T678/PKC |
| | | GROWTH FACTOR | L683 | |
| | | RECEPTOR (EC | | |
| | | 2.7.1.112). | | |
| LVEPLTPSGEAPN | B046-A | EPIDERMAL | L688 | T693/ |
| | | GROWTH FACTOR | N700 | ERK1, |
| | | RECEPTOR (EGFR) | | ERK2 |
| RELVEPLTPSGEA | B046-E | EPIDERMAL | R686 | T693/ |
| | | GROWTH FACTOR | A698 | ERK1, |
| | | RECEPTOR (EGFR) | | ERK2 |
| DSFLQRYSSDPTG | B046-B | EPIDERMAL | D1063 | S1070/ |
| | | GROWTH FACTOR | G1075 | CAM kinase |
| | | RECEPTOR (EGFR) | | I1; S1071/ |
| | | | | CAM kinase |
| | | | | I1 |
| STAENAEYLRV | B046-J | EPIDERMAL | S1190 | Y1197/ |
| AP | | GROWTH FACTOR | P1202 | autophosphorylation |
| | | RECEPTOR (EGFR) | | |
| FGMSRNLYAGD | DDR1_HUMAN | EPITHELIAL | F785 | Y792/ |
| YY | | DISCOIDIN DOMAIN | Y797 | autophosphorylation; |
| | | RECEPTOR 1 (EC | | Y796/ |
| | | 2.7.1.112) (TYROSINE- | | autophosphorylation; |
| | | PROTEIN KINASE | | Y797/ |
| | | CAK) (CELL | | autophosphorylation |
| | | ADHESION KINASE) | | |
| | | (TYROSINE KINASE | | |
| | | DDR) (DISCOIDIN | | |
| | | RECEPTOR | | |
| | | TYROSINE KINASE) | | |
| | | (TRK E) (PROTEIN- | | |
| | | TYRO INE | | |
| | | KINASE RTK 6). | | |
| LLLSNPAYRLLLA | DDR1_HUMAN | EPITHELIAL | L506 | Y513/ |
| | | DISCOIDIN DOMAIN | A518 | autophosphorylation |
| | | RECEPTOR 1 (EC | | |
| | | 2.7.1.112) (TYROSINE- | | |
| | | PROTEIN KINASE | | |
| | | CAK) (CELL | | |
| | | ADHESION KINASE) | | |
| | | (TYROSINE KINASE | | |
| | | DDR) (DISCOIDIN | | |
| | | RECEPTOR | | |
| | | TYROSINE KINASE) | | |
| | | (TRK E) (PROTEIN- | | |
| | | TYRO INE | | |
| | | KINASE RTK 6). | | |
| QQKIRKYTMRR | A066-A | ErbB-2 RECEPTOR | Q679 | T686 |
| LL | | PROTEIN-TYROSINE | L691 | |
| | | KINASE | | |
| PTAENPEYLGL | A066-D | ERBB-2 RECEPTOR | P1241 | Y1248/ |
| DV | | PROTEIN-TYROSINE | V1253 | EGFR |
| | | KINASE | | |
| QALDNPEYHNA | ERB4_HUMAN | ERBB-4 RECEPTOR | Q1181 | Y1188/ |
| SN | | PROTEIN-TYROSINE | N1193 | autophosphorylation |
| | | KINASE (EC | | |
| | | 2.7.1.112) (P180 ERB4) | | |
| | | (TYROSINE KINASE- | | |
| | | TYPE CELL | | |
| | | SURFACE RECEPTOR | | |
| | | HER4). | | |
| IVAENPEYLSEFS | ERB4_HUMAN | ERBB-4 RECEPTOR | I1277 | Y1284/ |
| | | PROTEIN-TYROSINE | S1289 | autophosphorylation |
| | | KINASE (EC | | |
| | | 2.7.1.112) (P180 ERB4) | | |
| | | (TYROSINE KINASE- | | |
| | | TYPE CELL | | |
| | | SURFACE RECEPTOR | | |
| | | HER4). | | |
| EKRHTRDSEAQ | STOM_HUMAN | ERYTHROCYTE | E2 L14 | S9/PKA |
| RL | | BAND 7 INTEGRAL | | |
| | | MEMBRANE | | |
| | | PROTEIN | | |
| | | (STOMATIN) | | |
| | | (PROTEIN 7.2B). | | |
| LLNKRRGSVPILR | B087-A | ERYTHROCYTE | L240 | S247/PKA |
| | | MEMBRANE | R252 | |
| | | PROTEIN BAND 4.2 | | |
| | | (P4.2) (PALLIDIN) | | |
| ASAASFEYTILDP | A041-B | ERYTHROPOIETIN | A419 | Y426/Jak2 |
| | | RECEPTOR (EPO-R) | P431 | |
| SEHAQDTYLVL | A041-A | ERYTHROPOIETIN | S361 | Y368/Jak2 |
| DK | | RECEPTOR (EPO-R) | K373 | |
| LHPPPQLSPFLQP | ESR1_HUMAN | ESTROGEN | L111 | S118/ |
| | | RECEPTOR (ER) | P123 | MAPK |
| | | (ESTRADIOL | | |
| | | RECEPTOR) (ER- | | |
| | | ALPHA). | | |
| GGRERLASTND | ESR1_HUMAN | ESTROGEN | G160 | S167/ |
| KG | | RECEPTOR (ER) | G172 | CDK2 |
| | | (ESTRADIOL | | |
| | | RECEPTOR) (ER- | | |
| | | ALPHA). | | |
| ISVDGLSTPVVLS | ELK1_HUMAN | ETS-DOMAIN | I410 | |
| | | PROTEIN ELK-1. | S422 | |
| LSTPVVLSPGPQK | ELK1_HUMAN | ETS-DOMAIN | L415 | |
| | | PROTEIN ELK-1. | K427 | |
| QAPGPALTPSLLP | ELK1_HUMAN | ETS-DOMAIN | Q346 | |
| | | PROTEIN ELK-1. | P358 | |
| IHFWSTLSPIAPR | ELK1_HUMAN | ETS-DOMAIN | I376 | S383/ |
| | | PROTEIN ELK-1. | R388 | p46SAPK, |
| | | | | p54SAPK |
| LLPTHTLTPVLLT | ELK1_HUMAN | ETS-DOMAIN | L356 | |
| | | PROTEIN ELK-1. | T368 | |
| GGPGPERTPGSGS | ELK1_HUMAN | ETS-DOMAIN | G329 | |
| | | PROTEIN ELK-1. | S341 | |
| TLTPVLLTPSSLP | ELK1_HUMAN | ETS-DOMAIN | T361 | |
| | | PROTEIN ELK-1. | P373 | |
| RDLELPLSPSLLG | ELK1_HUMAN | ETS-DOMAIN | R317 | |
| | | PROTEIN ELK-1. | G329 | |
| GEAGGPLTPRR | ERF_HUMAN | ETS-DOMAIN | G519 | T526/ |
| VS | | TRANSCRIPTION | S531 | MAPK1 |
| | | FACTOR ERF. | | |
| MILLSELSRRRIR | IF2A_HUMAN | EUKARYOTIC | M44 | S48/HRI, |
| | | TRANSLATION | R56 | HCR; S51/ |
| | | INITIATION FACTOR | | EIF2AK3, |
| | | 2 ALPHA SUBUNIT | | GCN2, |
| | | (EIF-2-ALPHA). | | HRI, PKR, |
| | | | | HCR, ds- |
| | | | | RNA kinase |
| DTATKSGSTTK | IF4E_HUMAN | EUKARYOTIC | D202 | S209/PKC, |
| NR | | TRANSLATION | R214 | Mnk1 |
| | | INITIATION FACTOR | | |
| | | 4E (EIF- | | |
| | | 4E) (EIF4E) (MRNA | | |
| | | CAP-BINDING | | |
| | | PROTEIN) (EIF-4F 25 KDA | | |
| | | SUBUNIT). | | |
| KEVHKSGYLSS | EZRI_HUMAN | EZRIN (P81) | K138 | Y145/ |
| ER | | (CYTOVILLIN) | R150 | PDGFR |
| | | (VILLIN-2). | | |
| LMLRLQDYEEK | EZRI_HUMAN | EZRIN (P81) | L346 | Y353/ |
| TK | | (CYTOVILLIN) | K358 | PDGFR |
| | | (VILLIN-2). | | |
| DSKNFDDYMKS | FABH_HUMAN | FATTY ACID- | D12 | Y19/TYR- |
| LG | | BINDING PROTEIN, | G24 | kinases |
| | | HEART (H-FABP) | | |
| | | (MUSCLE FATTY | | |
| | | ACID-BINDING | | |
| | | PROTEIN) (M-FABP) | | |
| | | (MAMMARY- | | |
| | | DERIVED GROWTH | | |
| | | INHIBITOR) (MDGI). | | |
| STTTTRRSCSKTV | B066-B | FIBRINOGEN | S453 | S460/ |
| | | | V465 | PKA, CK1 |
| EFPSRGKSSSYSK | B066-C | FIBRINOGEN | E569 | S576/PKC, |
| | | | K581 | CK1; S577/ |
| | | | | PKC, CK1; |
| | | | | S578/PKC, |
| | | | | CK1 |
| TLTTNEEYLDLSQ | A063-A | FIBROBLAST | T762 | Y769 |
| | | GROWTH FACTOR | Q774 | |
| | | RECEPTOR 2 (FGFR- | | |
| | | 2) | | |
| TVTSTDEYLDLSA | A062-A | FIBROBLAST | T753 | Y760/ |
| | | GROWTH FACTOR | A765 | autophosphorylation |
| | | RECEPTOR 3 (FGFR- | | |
| | | 3) | | |
| DVHNLDYYKKT | FGR3_HUMAN | FIBROBLAST | D641 | Y648/ |
| TN | | GROWTH FACTOR | N653 | autophosphorylation |
| | | RECEPTOR 3 (FGFR- | | |
| | | 3) (EC 2.7.1.112). | | |
| LLAVSEEYLDL | A061-A | FIBROBLAST | L747 | Y754/ |
| RL | | GROWTH FACTOR | L759 | autophosphorylation |
| | | RECEPTOR 4 (FGFR- | | |
| | | 4) | | |
| GVHHIDYYKKT | FGR4_HUMAN | FIBROBLAST | G636 | Y643/ |
| SN | | GROWTH FACTOR | N648 | autophosphorylation |
| | | RECEPTOR 4 (FGFR- | | |
| | | 4) (EC 2.7.1.112). | | |
| RYMEDSTYYKA | FAK1_HUMAN | FOCAL ADHESION | R569 | Y576/Src; |
| SK | | KINASE 1 (EC | K581 | Y577/Src |
| | | 2.7.1.112) (FADK 1) | | |
| | | (PP125FAK) | | |
| | | (PROTEIN- | | |
| | | TYROSINE KINASE | | |
| | | 2). | | |
| RYIEDEDYYKA | FAK2_HUMAN | FOCAL ADHESION | R572 | Y579/ |
| SV | | KINASE 2 (EC | V584 | autophosphorylation; |
| | | 2.7.1.112) (FADK 2) | | Y580/ |
| | | (PROLINE-RICH | | autophosphorylation |
| | | TYROSINE KINASE | | |
| | | 2) (CELL ADHESION | | |
| | | KINASE BETA) (CAK | | |
| | | BETA). | | |
| TFRPRTSSNASTI | FXO1_HUMAN | FORKHEAD PROTEIN | T312 | S319/PKB; |
| | | O1A (FORKHEAD IN | I324 | S322/PKB |
| | | RHABDOMYOSARCOMA). | | |
| QSRPRSCTWPL | FXO3_HUMAN | FORKHEAD PROTEIN | Q25 | T32/SGK1 |
| QR | | O3A (FORKHEAD IN | R37 | |
| | | RHABDOMYOSARCOMA- | | |
| | | LIKE 1) (AF6Q21 | | |
| | | PROTEIN). | | |
| LDIEQFSTVKGVN | GRK5_HUMAN | G PROTEIN- | L478 | S484/ |
| | | COUPLED RECEPTOR | N490 | autophosphorylation; |
| | | KINASE GRK5 (EC | | T485/ |
| | | 2.7.1.—). | | autophosphorylation |
| VLDIEQFSTVKGV | GRK6_HUMAN | G PROTEIN- | V477 | S484/ |
| | | COUPLED RECEPTOR | V489 | autophosphorylation; |
| | | KINASE GRK6 (EC | | T485/ |
| | | 2.7.1.—). | | autophosphorylation |
| PLPSGLLTPPQSG | CGE1_HUMAN | G1/S-SPECIFIC | P388 | T395/ |
| | | CYCLIN E1. | G400 | CDK2 |
| VCNGGIMTPPK | CGE2_HUMAN | G1/S-SPECIFIC | V385 | T392 |
| ST | | CYCLIN E2. | T397 | |
| FSLHDALSGSG | LEG3_HUMAN | GALECTIN-3 | F4 P16 | S5/CK1; |
| NP | | (GALACTOSE- | | S11/CK1 |
| | | SPECIFIC LECTIN 3) | | |
| | | (MAC-2 | | |
| | | ANTIGEN) (IGE- | | |
| | | BINDING PROTEIN) | | |
| | | (35 KDA LECTIN) | | |
| | | (CARBOHYDRATE | | |
| | | BINDING PROTEIN | | |
| | | 35) (CBP 35) | | |
| | | (LAMININ-BINDING | | |
| | | PROTEIN) (LECTIN L- | | |
| | | 29) (L-31) | | |
| | | (GALACTOSIDE | | |
| | | BINDING PROTEIN) | | |
| | | (GALBP). | | |
| SRLRRRASQLKIT | B164-A | GAMMA- | S427 | S434/PKC |
| | | AMINOBUTYRIC- | T439 | |
| | | ACID RECEPTOR | | |
| | | BETA-2 | | |
| | | SUBUNIT | | |
| FVSNRKPSKDK | B166-A | GAMMA- | F359 | S366/PKC |
| DK | | AMINOBUTYRIC- | K371 | |
| | | ACID RECEPTOR | | |
| | | GAMMA-2S SUBUNIT | | |
| ITSTLASSFKRRS | NMZ1_HUMAN | GLUTAMATE | I883 | S889/PKC; |
| | | [NMDA] RECEPTOR | R895 | S890/PKC |
| | | SUBUNIT ZETA 1 | | |
| | | (NR1). | | |
| AITSTLASSFKRR | NMZ1_HUMAN | GLUTAMATE | A882 | S889/PKC; |
| | | [NMDA] RECEPTOR | R894 | S890/PKC |
| | | SUBUNIT ZETA 1 | | |
| | | (NR1). | | |
| TMTFFKKSKISTY | B170-A | GLUTAMATE | T690 | S697/PKA |
| | | RECEPTOR 6 (GLUR- | Y702 | (minor site) |
| | | 6) (GLUTAMATE | | |
| | | RECEPTOR, | | |
| FMSSRRQSVLV | B170-B | GLUTAMATE | F708 | S715/PKA |
| KS | | RECEPTOR 6 (GLUR- | S720 | (major site) |
| | | 6) (GLUTAMATE | | |
| | | RECEPTOR, | | |
| EKMWAFMSSR | GLK1_HUMAN | GLUTAMATE | E718 | S725/PKC |
| QQT | | RECEPTOR, | T730 | |
| | | IONOTROPIC | | |
| | | KAINATE 1 | | |
| | | (GLUTAMATE | | |
| | | RECEPTOR 5) (GLUR- | | |
| | | 5) (EXCITATORY | | |
| | | AMINO | | |
| | | ACID RECEPTOR 3) | | |
| | | (EAA3). | | |
| STSIEYVTQRNCN | GLK1_HUMAN | GLUTAMATE | S754 | T761/PKC |
| | | RECEPTOR, | N766 | |
| | | IONOTROPIC | | |
| | | KAINATE 1 | | |
| | | (GLUTAMATE | | |
| | | RECEPTOR 5) (GLUR- | | |
| | | 5) (EXCITATORY | | |
| | | AMINO | | |
| | | ACID RECEPTOR 3) | | |
| | | (EAA3). | | |
| EREGSKRYCIQTK | AMPE_HUMAN | GLUTAMYL | E5 K17 | Y12 |
| | | AMINOPEPTIDASE | | |
| | | (EC 3.4.11.7) (EAP) | | |
| | | (AMINOPEPTIDASE | | |
| | | A) (APA) | | |
| | | (DIFFERENTIATION | | |
| | | ANTIGEN | | |
| | | GP160). | | |
| MLRGRSLSVTS | GYS2_HUMAN | GLYCOGEN | M1 G13 | S8/PKA; |
| LG | | [STARCH] | | S11 |
| | | SYNTHASE, LIVER | | |
| | | (EC 2.4.1.11). | | |
| FKYPRPSSVPPSP | GYS2_HUMAN | GLYCOGEN | F634 | S641; S645 |
| | | [STARCH] | P646 | |
| | | SYNTHASE, LIVER | | |
| | | (EC 2.4.1.11). | | |
| QASSPQSSDVEDE | GYS2_HUMAN | GLYCOGEN | Q650 | S653; S657 |
| | | [STARCH] | E662 | |
| | | SYNTHASE, LIVER | | |
| | | (EC 2.4.1.11). | | |
| PSGSQASSPQSSD | GYS2_HUMAN | GLYCOGEN | P646 | S649; S653; |
| | | [STARCH] | D658 | S657 |
| | | SYNTHASE, LIVER | | |
| | | (EC 2.4.1.11). | | |
| PSLSRHSSPHQSE | GYS1_HUMAN | GLYCOGEN | P646 | S647/CK1; |
| | | [STARCH] | E658 | S649/ |
| | | SYNTHASE, MUSCLE | | GSK-3; |
| | | (EC 2.4.1.11). | | S652/CK1 |
| | | | | S653/CK1; |
| | | | | S657 |
| NRTLSMSSLPGLE | GYS1_HUMAN | GLYCOGEN | N4 E16 | S8/PKA; |
| | | [STARCH] | | S10/CK1; |
| | | SYNTHASE, MUSCLE | | S11 |
| | | (EC 2.4.1.11). | | |
| RPASVPPSPSLSR | GYS1_HUMAN | GLYCOGEN | R638 | S641/ |
| | | [STARCH] | R650 | GSK-3; |
| | | SYNTHASE, MUSCLE | | S645/ |
| | | (EC 2.4.1.11). | | GSK- |
| | | | | 3; S647/ |
| | | | | CK1; S649/ |
| | | | | GSK-3 |
| TSGSKRNSVDT | GYS1_HUMAN | GLYCOGEN | T703 | S710/ |
| AT | | [STARCH] | T715 | PKA, CK1; |
| | | SYNTHASE, MUSCLE | | T713/CK1 |
| | | (EC 2.4.1.11). | | |
| MPLNRTLSMSS | GYS1_HUMAN | GLYCOGEN | M1 P13 | S8/PKA; |
| LP | | [STARCH] | | S10/CK1; |
| | | SYNTHASE, MUSCLE | | S11 |
| | | (EC 2.4.1.11). | | |
| SEKRKQISVRGLA | PHS3_HUMAN | GLYCOGEN | S8 A20 | S15/PHK |
| | | PHOSPHORYLASE, | | (in |
| | | BRAIN FORM (EC | | Phosphorylase |
| | | 2.4.1.1). | | A) |
| QEKRRQISIRGIV | PHS1_HUMAN | GLYCOGEN | Q8 V20 | S15/PHK |
| | | PHOSPHORYLASE, | | (in |
| | | LIVER FORM (EC | | Phosphorylase |
| | | 2.4.1.1). | | A) |
| SGRPRTTSFAESC | KG3B_HUMAN | GLYCOGEN | S2 C14 | S9 PKA/ |
| | | SYNTHASE KINASE-3 | | PKB |
| | | BETA (EC 2.7.1.37) | | |
| | | (GSK- | | |
| | | 3 BETA). | | |
| RGEPNVSYICSRY | KG3B_HUMAN | GLYCOGEN | R209 | Y216 |
| | | SYNTHASE KINASE-3 | Y221 | |
| | | BETA (EC 2.7.1.37) | | |
| | | (GSK- | | |
| | | 3 BETA). | | |
| KKPRRKDTPAL | GSUB_HUMAN | G-SUBSTRATE. | K61 I73 | T68/PKA, |
| HI | | | | PKB |
| FERASEYQLND | GBT1_HUMAN | GUANINE | F135 | Y141/Src |
| SA | | NUCLEOTIDE- | A147 | |
| | | BINDING PROTEIN | | |
| | | G(T), ALPHA-1 | | |
| | | SUBUNIT | | |
| | | (TRANSDUCIALPHA- | | |
| | | 1 CHAIN) | | |
| PAYSRALSRQLSS | B118-B | HEAT SHOCK 27 KD | P71 S83 | S78/ |
| | | PROTEIN (HSP 27) | | MAPKAPK |
| | | | | 2; S82/ |
| | | | | MAPKAPK |
| | | | | 2, PKC, |
| | | | | PKA |
| FSLLRGPSWDPFR | HS27_HUMAN | HEAT SHOCK 27 KDA | F8 R20 | S15/ |
| | | PROTEIN (HSP 27) | | MAPKAPK |
| | | (STRESS- | | 2, PKC, |
| | | RESPONSIVE | | PKA |
| | | PROTEIN 27) (SRP27) | | |
| | | (ESTROGEN- | | |
| | | REGULATED 24 KDA | | |
| | | PROTEIN) (28 KDA | | |
| | | HEAT SHOCK | | |
| | | PROTEIN). | | |
| RALSRQLSSGVSE | HS27_HUMAN | HEAT SHOCK 27 KDA | R75 E87 | S78/ |
| | | PROTEIN (HSP 27) | | MAPKAPK |
| | | (STRESS- | | 2; S82/ |
| | | RESPONSIVE | | MAPKAPK |
| | | PROTEIN 27) (SRP27) | | 2, PKC, |
| | | (ESTROGEN- | | PKA |
| | | REGULATED 24 KDA | | |
| | | PROTEIN) (28 KDA | | |
| | | HEAT SHOCK | | |
| | | PROTEIN). | | |
| PKIEDVGSDEEDD | HS9B_HUMAN | HEAT SHOCK | P247 | S254/CK2 |
| | | PROTEIN HSP 90- | D259 | |
| | | BETA (HSP 84) (HSP | | |
| | | 90). | | |
| KEREKEISDDEAE | HS9B_HUMAN | HEAT SHOCK | K218 | S225/CK2 |
| | | PROTEIN HSP 90- | E230 | |
| | | BETA (HSP 84) (HSP | | |
| | | 90). | | |
| PHLDRLVSARS | A009-A | HEPATOCYTE | P978 | S985/PKC |
| VS | | GROWTH FACTOR | S990 | |
| | | RECEPTOR (HGF-SF | | |
| | | RECEPTOR) | | |
| RDMYDKEYYS | A009-B | HEPATOCYTE | R1227 | Y1230/ |
| VHN | | GROWTH FACTOR | N1239 | autophosphorylation; |
| | | RECEPTOR (HGF-SF | | Y1234/ |
| | | RECEPTOR) | | autophosphorylation; |
| | | | | Y1235/ |
| | | | | autophosphorylation |
| YVHVNATYVN | A009-D | HEPATOCYTE | Y1349 | Y1349/ |
| VKC | | GROWTH FACTOR | C1361 | autophosphorylation; |
| | | RECEPTOR (HGF-SF | | Y1356/ |
| | | RECEPTOR) | | autophosphorylation |
| DMYDKEYYSV | MET_HUMAN | HEPATOCYTE | D1228 | Y1230/ |
| HNK | | GROWTH FACTOR | K1240 | autophosphorylation; |
| | | RECEPTOR (MET | | Y1234/ |
| | | PROTO-ONCOGENE | | autophosphorylation; |
| | | TYROSINE KINASE) | | Y1235/ |
| | | (EC 2.7.1.112) | | autophosphorylation |
| | | (HGF-SF RECEPTOR). | | |
| HIIENPQYFSDAC | TRKA_HUMAN | HIGH AFFINITY | H489 | Y496/ |
| | | NERVE GROWTH | C501 | autophosphorylation |
| | | FACTOR RECEPTOR | | |
| | | (EC | | |
| | | 2.7.1.112) (TRK1 | | |
| | | TRANSFORMING | | |
| | | TYROSINE KINASE | | |
| | | PROTEIN) (P140- | | |
| | | TRKA) (TRK-A). | | |
| KEEEEGISQESSE | HMGI_HUMAN | HIGH MOBILITY | K91 | S98/CK2; |
| | | GROUP PROTEIN | E103 | S101/CK2; |
| | | HMG-Y. | | S102/CK2 |
| FESERRGSHPYID | CN7A_HUMAN | HIGH-AFFINITY | F77 D89 | S84 |
| | | CAMP-SPECIFIC 3′,5′- | | |
| | | CYCLIC | | |
| | | PHOSPHODIESTERASE | | |
| | | (EC 3.1.4.17) (HCP1) | | |
| | | (TM22). | | |
| ISMISADSHEKRH | HIS1_HUMAN | HISTATIN 1 | I14 H26 | S21 |
| | | (HISTIDINE-RICH | | |
| | | PROTEIN 1) (POST-PB | | |
| | | PROTEIN) (PPB) | | |
| | | [CONTAINS: | | |
| | | HISTATIN 2]. | | |
| AKAKTRSSRAG | B025-B | HISTONE H2A.1 | A12 | S19/PKA |
| LQ | | | Q24 | |
| RKRSRKESYSV | B014-C | HISTONE H2B | R29 | S32/PKA, |
| YV | | | V41 | PKG, PKC, |
| | | | | histone |
| | | | | kinase; S36/ |
| | | | | PKA, |
| | | | | PKC, PKG |
| DGKKRKRSRKE | B014-B | HISTONE H2B | D25 | S32/PKA, |
| SY | | | Y37 | PKG, PKC, |
| | | | | histone |
| | | | | kinase; S36/ |
| | | | | PKA, |
| | | | | PKC, PKG |
| APAPKKGSKKA | B014-A | HISTONE H2B | A7 T19 | S14/PKA |
| VT | | | | (Mst1 |
| | | | | kinase) |
| RGGVKRISGLIYE | B059-B | HISTONE H4 | R40 E52 | S47/H4 |
| | | | | PK1 |
| SDRKGGSYSQA | B259-A | HLA CLASS I | S337 | Y344/ |
| AS | | HISTOCOMPATIBILITY | S349 | |
| | | ANTIGEN, A-2 | | |
| | | ALPHA CHAIN | | |
| IAEPMRRSVSEAA | LIPS_HUMAN | HORMONE | I545 | S552/ |
| | | SENSITIVE LIPASE | A557 | PKA; S554/ |
| | | (EC 3.1.1.—) (HSL). | | AMPK |
| YASSNPEYLSASD | A040-E | INSULIN RECEPTOR | Y992 | Y992/ |
| | | | D1004 | autophosphorylation; |
| | | | | Y999/ |
| | | | | autophosphorylation |
| SYEEHIPYTHM | B141-I | INSULIN RECEPTOR | S1354 | S1354/ |
| NG | | (IR) | G1366 | INSR |
| | | | | (autophosphorylation); |
| | | | | Y1355/ |
| | | | | INSR |
| | | | | (autophosphorylation); |
| | | | | Y1361/ |
| | | | | INSR |
| | | | | (autophosphorylation) |
| SSLGFKRSYEEHI | B141-C | INSULIN RECEPTOR | S1347 | S1354/ |
| | | (IR) | I1359 | INSR |
| | | | | (autophosphorylation); |
| | | | | Y1355/ |
| | | | | INSR |
| | | | | (autophosphorylation) |
| KKNGRILTLPRSN | B141-D | INSULIN RECEPTOR | K1368 | S1375/ |
| | | (IR) | N1380 | PKC |
| ENVPLDRSSHC | B141-A | INSULIN RECEPTOR | E1325 | S1332/ |
| QR | | (IR) | R1337 | insulin- |
| | | | | sensitive |
| | | | | serine |
| | | | | kinase |
| | | | | (IRSK); |
| | | | | S1333/ |
| | | | | insulin- |
| | | | | sensitive |
| | | | | serine |
| | | | | kinase |
| | | | | (IRSK) |
| SLGFKRSYEEHIP | B141-H | INSULIN RECEPTOR | S1348 | S1354/ |
| | | (IR) | P1360 | INSR |
| | | | | (autophosphorylation); |
| | | | | Y1355/ |
| | | | | INSR |
| | | | | (autophosphorylation) |
| EETGTEEYMKM | IRS1_HUMAN | INSULIN RECEPTOR | E934 | Y941/ |
| DL | | SUBSTRATE-1 (IRS- | L946 | INSR |
| | | 1). | | |
| GRKGSGDYMP | IRS1_HUMAN | INSULIN RECEPTOR | G625 | Y632 INSR |
| MSP | | SUBSTRATE-1 (IRS- | P637 | |
| | | 1). | | |
| GEEELSNYICM | IRS1_HUMAN | INSULIN RECEPTOR | G458 | Y465/ |
| GG | | SUBSTRATE-1 (IRS- | G470 | INSR |
| | | 1). | | |
| VPSSRGDYMTM | IRS1_HUMAN | INSULIN RECEPTOR | V982 | Y989/ |
| QM | | SUBSTRATE-1 (IRS- | M994 | INSR |
| | | 1). | | |
| GSCRSDDYMPM | IRS2_HUMAN | INSULIN RECEPTOR | G668 | Y675/ |
| SP | | SUBSTRATE-2 (IRS- | P680 | INSR |
| | | 2). | | |
| EPKSPGEYINIDF | IRS2_HUMAN | INSULIN RECEPTOR | E912 | Y919/ |
| | | SUBSTRATE-2 (IRS- | F924 | INSR |
| | | 2). | | |
| GGGGGEFYGY | IRS2_HUMAN | INSULIN RECEPTOR | G533 | Y540/ |
| MTM | | SUBSTRATE-2 (IRS- | M545 | INSR |
| | | 2). | | |
| RSPLSDYMNLD | IRS2_HUMAN | INSULIN RECEPTOR | R972 | Y978/ |
| FS | | SUBSTRATE-2 (IRS- | S984 | INSR |
| | | 2). | | |
| LAKAQETSGEEIS | IBP1_HUMAN | INSULIN-LIKE | L187 | S194 |
| | | GROWTH FACTOR | S199 | |
| | | BINDING PROTEIN 1 | | |
| | | (IGFBP-1) (IBP-1) | | |
| | | (IGF-BINDING | | |
| | | PROTEIN 1) | | |
| | | (PLACENTAL | | |
| | | PROTEIN 12) (PP12). | | |
| GSPESPESTEITE | IBP1_HUMAN | INSULIN-LIKE | G119 | S126 |
| | | GROWTH FACTOR | E131 | |
| | | BINDING PROTEIN 1 | | |
| | | (IGFBP-1) (IBP-1) | | |
| | | (IGF-BINDING | | |
| | | PROTEIN 1) | | |
| | | (PLACENTAL | | |
| | | PROTEIN 12) (PP12). | | |
| NFHLMAPSEED | IBP1_HUMAN | INSULIN-LIKE | N137 | S144 |
| HS | | GROWTH FACTOR | S149 | |
| | | BINDING PROTEIN 1 | | |
| | | (IGFBP-1) (IBP-1) | | |
| | | (IGF-BINDING | | |
| | | PROTEIN 1) | | |
| | | (PLACENTAL | | |
| | | PROTEIN 12) (PP12). | | |
| FHLMAPSEEDH | IBP1_HUMAN | INSULIN-LIKE | F138 | S144 |
| SI | | GROWTH FACTOR | I150 | |
| | | BINDING PROTEIN 1 | | |
| | | (IGFBP-1) (IBP-1) | | |
| | | (IGF-BINDING | | |
| | | PROTEIN 1) | | |
| | | (PLACENTAL | | |
| | | PROTEIN 12) (PP12). | | |
| KQDSNPLYKSAIT | ITB7_HUMAN | INTEGRIN BETA-7 | K771 | Y778/ |
| | | | T783 | TYR- |
| | | | | kinases; |
| | | | | T783/PK |
| PSSSIDEYFSEQP | INR1_HUMAN | INTERFERON- | P474 | Y481/ |
| | | ALPHA/BETA | P486 | p135TYK2 |
| | | RECEPTOR ALPHA | | |
| | | CHAIN (IFN-ALPHA- | | |
| | | REC). | | |
| VFLRCINYVFFPS | INR1_HUMAN | INTERFERON- | V459 | Y466/ |
| | | ALPHA/BETA | S471 | p135TYK2 |
| | | RECEPTOR ALPHA | | |
| | | CHAIN (IFN-ALPHA- | | |
| | | REC). | | |
| SLPDHKKTLEH | IL7R_HUMAN | INTERLEUKIN-7 | S275 | T282/PKC |
| LC | | RECEPTOR ALPHA | C287 | |
| | | CHAIN (IL-7R- | | |
| | | ALPHA) (CDW127) | | |
| | | (CD127 ANTIGEN). | | |
| GAGFGSRSLYG | K2CF_HUMAN | KERATIN, TYPE II | G52 | S59 |
| LG | | CYTOSKELETAL 6F | G64 | |
| | | (CYTOKERATIN 6F) | | |
| | | (CK 6F) (K6F | | |
| | | KERATIN). | | |
| SPVFTSRSAAFSG | K2C7_HUMAN | KERATIN, TYPE II | S6 G18 | S13 |
| | | CYTOSKELETAL 7 | | |
| | | (CYTOKERATIN 7) | | |
| | | (K7) (CK 7). | | |
| PRAFSSRSYTSGP | K2C8_HUMAN | KERATIN, TYPE II | P16 P28 | S23 |
| | | CYTOSKELETAL 8 | | |
| | | (CYTOKERATIN 8) | | |
| | | (K8) (CK 8). | | |
| SAYGGLTSPGLSY | K2C8_HUMAN | KERATIN, TYPE II | S424 | S431/ |
| | | CYTOSKELETAL 8 | Y436 | MAPK, |
| | | (CYTOKERATIN 8) | | CAMK2 |
| | | (K8) (CK 8). | | |
| LDIPTGTTPQRKS | EG5_HUMAN | KINESIN-RELATED | L920 | T927/ |
| | | MOTOR PROTEIN | S932 | p34cdc2 |
| | | EG5 (KINESIN-LIKE | | |
| | | SPINDLE PROTEIN | | |
| | | HKSP) (THYROID | | |
| | | RECEPTOR | | |
| | | INTERACTING | | |
| | | PROTEIN 5) (TRIP5). | | |
| SGAQASSTPLSPT | B176-H | LAMIN A (70 KD | S12 T24 | T 19/cdc2; |
| | | LAMIN) | | S22/cdc2 |
| DAENRLQTMKE | B176-I | LAMIN A (70 KD | D192 | T199/PKC |
| EL | | LAMIN) | L204 | |
| NGDDPLLTYRF | B176-K | LAMIN A (70 KD | N473 | T480/PKC |
| PP | | LAMIN) | P485 | |
| QASSTPLSPTRIT | B176-B | LAMIN A (70 KD | Q15 T27 | T19/cdc2; |
| | | LAMIN) | | S22/cdc2 |
| NTWGCGNSLRT | B176-F | LAMIN A (70 kD | N518 | S525/PKC |
| AL | | LAMIN) | L530 | |
| TQGGGSVTKKR | B176-J | LAMIN A (70 KD | T409 | T416/PKC |
| KL | | LAMIN) | L421 | |
| QRSRGRASSHSSQ | B176-D | LAMIN A (70 kD | Q396 | S403/PKC; |
| | | LAMIN) | Q408 | S404/PKC |
| SSVTVTRSYRSVG | B176-G | LAMIN A (70 kD | S618 | S625/PKC |
| | | LAMIN) | G630 | |
| ERLRLSPSPTSQR | B176-C | LAMIN A (70 kD | E385 | S392/cdc2 |
| | | LAMIN) | R397 | |
| TVSRASSSRSVRT | B008-D | LAMIN B1 | T398 | S404/beta |
| | | | T410 | II PKC |
| ERLKLSPSPSSRV | B008-B | LAMIN B1 | E385 | S392/cdc2 |
| | | | V397 | kinase; |
| | | | | S394/ |
| | | | | beta II PKC |
| GGPTTPLSPTRLS | B008-A | LAMIN B1 | G15 S27 | S22/cdc2 |
| | | | | kinase |
| KLSPSPSSRVTVS | B008-C | LAMIN B1 | K388 | S392/cdc2 |
| | | | S400 | kinase; |
| | | | | S394/ |
| | | | | beta II PKC |
| KSISERLSVLKGA | MIP_HUMAN | LENS FIBER MAJOR | K228 | S229/ |
| | | INTRINSIC PROTEIN | A240 | cAMP- |
| | | (MIP26) (MP26) | | dependent |
| | | (AQUAPORIN 0). | | protein |
| | | | | kinase; |
| | | | | S231/ |
| | | | | cAMP- |
| | | | | dependent |
| | | | | protein |
| | | | | kinase; |
| | | | | S235 |
| KGAKPDVSNGQ | MIP_HUMAN | LENS FIBER MAJOR | K238 | |
| PE | | INTRINSIC PROTEIN | E250 | |
| | | (MIP26) (MP26) | | |
| | | (AQUAPORIN 0). | | |
| RDIYRASYYRR | KLTK_HUMAN | LEUKOCYTE | R669 | |
| GD | | TYROSINE KINASE | D681 | |
| | | RECEPTOR (EC | | |
| | | 2.7.1.112). | | |
| GKKTKFASDDE | LA_HUMAN | LUPUS LA PROTEIN | G359 | T362; S366/ |
| HD | | (SJOGREN | D371 | CK2 |
| | | SYNDROME TYPE B | | |
| | | ANTIGEN (SS-B)) (LA | | |
| | | RIBONUCLEOPROTEIN) | | |
| | | (LA | | |
| | | AUTOANTIGEN). | | |
| SSQGVDTYVEM | A072-C | MACROPHAGE | S716 | Y723 |
| RP | | COLONY | P728 | |
| | | STIMULATING | | |
| | | FACTOR 1 | | |
| | | RECEPTOR | | |
| NIHLEKKYVRR | KFMS_HUMAN | MACROPHAGE | N701 | Y708 |
| DS | | COLONY | S713 | |
| | | STIMULATING | | |
| | | FACTOR I RECEPTOR | | |
| | | (CSF-1-R) (EC | | |
| | | 2.7.1.112) (FMS | | |
| | | PROTO- | | |
| | | ONCOGENE) (C-FMS) | | |
| | | (CD115). | | |
| YVQLPATYMNL | RON_HUMAN | MACROPHAGE- | Y1353 | Y1353/ |
| GP | | STIMULATING | P1365 | Ron; Y1360/ |
| | | PROTEIN RECEPTOR | | Ron |
| | | (EC | | |
| | | 2.7.1.112) (MSP | | |
| | | RECEPTOR) (P185- | | |
| | | RON) (CDW136) | | |
| | | (CD136 | | |
| | | ANTIGEN). | | |
| SALLGDHYVQL | RON_HUMAN | MACROPHAGE- | S1346 | Y1353/ |
| PA | | STIMULATING | A1358 | Ron |
| | | PROTEIN RECEPTOR | | |
| | | (EC | | |
| | | 2.7.1.112) (MSP | | |
| | | RECEPTOR) (P185- | | |
| | | RON) (CDW136) | | |
| | | (CD136 | | |
| | | ANTIGEN). | | |
| DSMKDEEYEQ | MAD3_HUMAN | MAJOR | D35 | S36/ |
| MVK | | HISTOCOMPATIBILITY | K47 | IKKA, |
| | | COMPLEX | | IKKB, IKKE; |
| | | ENHANCER- | | Y42/ |
| | | BINDING PROTEIN | | Tyr- |
| | | MAD3 (NUCLEAR | | kinases |
| | | FACTOR KAPPA-B | | |
| | | INHIBITOR) (I- | | |
| | | KAPPA-B-ALPHA) | | |
| | | (IKBA). | | |
| QSTKVPQTPLHTS | MKK2_HUMAN | MAP KINASE- | Q327 | T334/ |
| | | ACTIVATED | S339 | MAPK; |
| | | PROTEIN KINASE 2 | | T338/ |
| | | (EC 2.7.1.—) (MAPK- | | autophosphorylation |
| | | ACTIVATED | | (likely) |
| | | PROTEIN KINASE 2) | | |
| | | (MAPKAP KINASE 2) | | |
| | | (MAPKAPK-2). | | |
| PFKLSGLSFKRNR | MRP_HUMAN | MARCKS-RELATED | P96 | S100/PKC; |
| | | PROTEIN (MAC- | R108 | S103/PKC |
| | | MARCKS). | | |
| DIKNDSNYVVK | KKIT_HUMAN | MAST/STEM CELL | D816 | S821/PKC |
| GN | | GROWTH FACTOR | N828 | alpha |
| | | RECEPTOR (EC | | |
| | | 2.7.1.112) (SCFR) | | |
| | | (PROTO-ONCOGENE | | |
| | | TYROSINE-PROTEIN | | |
| | | KINASE KIT) (C-KIT) | | |
| | | (CD117 ANTIGEN). | | |
| CSDSTNEYMDM | A065-D | MAST/STEM CELL | C714 | Y721/ |
| KP | | GROWTH FACTOR | P726 | CHK |
| | | RECEPTOR (SCFR) | | |
| ESHESMESYEL | MGP_HUMAN | MATRIX GLA- | E21 P33 | S22; S25; |
| NP | | PROTEIN (MGP). | | S28 |
| VVTLCYESHES | MGP_HUMAN | MATRIX GLA- | V15 E27 | S22; S25; |
| ME | | PROTEIN (MGP). | | |
| LCYESHESMES | MGP_HUMAN | MATRIX GLA- | L18 E30 | S22; S25; |
| YE | | PROTEIN (MGP). | | S28 |
| GSRSRTPSLPTPP | B154-C | MICROTUBULE- | G523 | S524/ |
| | | ASSOCIATED | P535 | GSK3; |
| | | PROTEIN TAU | | S526; T528/ |
| | | | | MAPK, GSK3; |
| | | | | S530/ |
| | | | | PKA, |
| | | | | CDK5, |
| | | | | PKC; T533/ |
| | | | | CDK5, |
| | | | | GSK3 |
| VDLSKVTSKCG | B154-F | MICROTUBULE- | V629 | S640/ |
| SL | | ASSOCIATED | L641 | MARK1, |
| | | PROTEIN TAU | | GSK3 |
| | | | | ALPHA |
| GAEIVYKSPVVSG | B154-I | MICROTUBULE- | G705 | S712/ |
| | | ASSOCIATED | G717 | PDPK, |
| | | PROTEIN TAU | | MAPK, |
| | | | | GSK3, |
| | | | | CDK5; |
| | | | | S716/ |
| | | | | GSK3 |
| PVVSGDTSPRHLS | B154-J | MICROTUBULE- | P713 | S716/ |
| | | ASSOCIATED | S725 | GSK3; |
| | | PROTEIN TAU | | T719/ |
| | | | | GSK- |
| | | | | 3Beta; S720/ |
| | | | | MAPK, |
| | | | | GSK3, |
| | | | | CDK5, |
| | | | | PDPK; |
| | | | | S725/PKA |
| DTSPRHLSNVSST | B154-K | MICROTUBULE- | D718 | T719/ |
| | | ASSOCIATED | T730 | GSK-3Beta; |
| | | PROTEIN TAU | | S720/ |
| | | | | MAPK, |
| | | | | GSK3, |
| | | | | CDK5, |
| | | | | PDPK; |
| | | | | S725/ |
| | | | | PKA; S728/; |
| | | | | S729/ |
| | | | | GSK- |
| | | | | 3BETA |
| RVQSKIGSLDNIT | B154-H | MICROTUBULE- | R665 | S672/ |
| | | ASSOCIATED | T677 | MARK1, |
| | | PROTEIN TAU | | PKA, CaM |
| | | | | Kinase II, |
| | | | | C-Kinase, |
| | | | | GSK3, |
| | | | | PKA p110k |
| TPGSRSRTPSLPT | B154-P | MICROTUBULE- | T521 | T521/ |
| | | ASSOCIATED | T533 | PDPK, |
| | | PROTEIN TAU | | CDK5, |
| | | | | GSK3Beta; |
| | | | | S524/ |
| | | | | GSK3; |
| | | | | S526/; |
| | | | | T528/ |
| | | | | PDPK, |
| | | | | MAPK, |
| | | | | GSK3, |
| | | | | CDK5, |
| | | | | PKA; S530/ |
| | | | | PKA, |
| | | | | CDK5, PKC; |
| | | | | T533/ |
| | | | | PDPK, |
| | | | | CDK5, |
| | | | | GSK-3 |
| RHLSNVSSTGSID | B154-L | MICROTUBULE- | R722 | S725/ |
| | | ASSOCIATED | D734 | PKA; S728/; |
| | | PROTEIN TAU | | S729/ |
| | | | | GSK- |
| | | | | 3BETA; |
| | | | | S732/ |
| | | | | CaMK2 |
| SNVSSTGSIDMVD | B154-M | MICROTUBULE- | S725 | S725/ |
| | | ASSOCIATED | D737 | PKA; S728/; |
| | | PROTEIN TAU | | S729/ |
| | | | | GSK- |
| | | | | 3BETA; |
| | | | | S732/ |
| | | | | CaMK2 |
| KKVAVVRTPPK | B154-Q | MICROTUBULE- | K540 | T547/ |
| SP | | ASSOCIATED | P552 | PDPK, |
| | | PROTEIN TAU | | GSK3, |
| | | | | CDK5 |
| | | | | A-Kinase; |
| | | | | S551/ |
| | | | | PDPK, |
| | | | | MAPK, |
| | | | | GSK-3, |
| | | | | CDK5 |
| KVTSKCGSLGNIH | B154-G | MICROTUBULE- | K633 | S640/ |
| | | ASSOCIATED | H645 | MARK1, |
| | | PROTEIN TAU | | GSK3 |
| | | | | ALPHA |
| VVRTPPKSPSSAK | B154-D | MICROTUBULE- | V544 | T547/ |
| | | ASSOCIATED | K556 | PDPK, |
| | | PROTEIN TAU | | GSK3, |
| | | | | CDK5 |
| | | | | A-Kinase; |
| | | | | S551/ |
| | | | | PDPK, |
| | | | | MAPK, |
| | | | | GSK-3, |
| | | | | CDK5; |
| | | | | S553/ |
| | | | | PK; S554 |
| GDRSGYSSPGSPG | B154-A | MICROTUBULE- | G508 | S511/A- |
| | | ASSOCIATED | G520 | kinase, |
| | | PROTEIN TAU | | GSK-3; |
| | | | | S514/ |
| | | | | PDPK, A- |
| | | | | kinase gsk- |
| | | | | 3; S514 |
| | | | | Dephosphorylierung: |
| | | | | Protein |
| | | | | phosphatase |
| | | | | 2, |
| | | | | regulatory |
| | | | | subunit B |
| | | | | (B56), alpha |
| | | | | protein |
| | | | | phosphatase |
| | | | | 5; S515/ |
| | | | | PDPK, |
| | | | | MAPK, |
| | | | | GSK-3, A- |
| | | | | kinase; |
| | | | | S515 |
| | | | | Dephosphorylierung: |
| | | | | Protein |
| | | | | phosphatase |
| | | | | 2, |
| | | | | regulatory |
| | | | | subunit B |
| | | | | (B56), alpha |
| | | | | protein |
| | | | | phosphatase |
| | | | | 5; |
| | | | | S518/ |
| | | | | PDPK, |
| | | | | MAPK, |
| | | | | CDK5, |
| | | | | GSK-3 beta |
| SGYSSPGSPGTPG | B154-B | MICROTUBULE- | S511 | S511/A- |
| | | ASSOCIATED | G523 | kinase, |
| | | PROTEIN TAU | | GSK-3; |
| | | | | S514/ |
| | | | | PDPK, A- |
| | | | | kinase gsk- |
| | | | | 3; S514 |
| | | | | Dephosphorylierung: |
| | | | | Protein |
| | | | | phosphatase |
| | | | | 2, |
| | | | | regulatory |
| | | | | subunit B |
| | | | | (B56), alpha |
| | | | | protein |
| | | | | phosphatase |
| | | | | 5; S515/ |
| | | | | PDPK, |
| | | | | MAPK, |
| | | | | GSK-3, A- |
| | | | | kinase; |
| | | | | S515 |
| | | | | Dephosphorylierung: |
| | | | | Protein |
| | | | | phosphatase |
| | | | | 2, |
| | | | | regulatory |
| | | | | subunit B |
| | | | | (B56), alpha |
| | | | | protein |
| | | | | phosphatase |
| | | | | 5; |
| | | | | S518/ |
| | | | | PDPK, |
| | | | | MAPK, |
| | | | | CDK5, |
| | | | | GSK-3 beta; |
| | | | | T521/ |
| | | | | PDPK, |
| | | | | CDK5, |
| | | | | GSK3Beta |
| SSPGSPGTPGSRS | B154-O | MICROTUBULE- | S514 | S514/ |
| | | ASSOCIATED | S526 | PDPK, A- |
| | | PROTEIN TAU | | kinase gsk- |
| | | | | 3; S514 |
| | | | | Dephosphorylierung: |
| | | | | Protein |
| | | | | phosphatase |
| | | | | 2, |
| | | | | regulatory |
| | | | | subunit B |
| | | | | (B56), alpha |
| | | | | protein |
| | | | | phosphatase |
| | | | | 5; S515/ |
| | | | | PDPK, |
| | | | | MAPK, |
| | | | | GSK-3, A- |
| | | | | kinase; |
| | | | | S515 |
| | | | | Dephosphorylierung: |
| | | | | Protein |
| | | | | phosphatase |
| | | | | 2, |
| | | | | regulatory |
| | | | | subunit B |
| | | | | (B56), alpha |
| | | | | protein |
| | | | | phosphatase |
| | | | | 5; |
| | | | | S518/ |
| | | | | PDPK, |
| | | | | MAPK, |
| | | | | CDK5, |
| | | | | GSK-3 beta; |
| | | | | T521/ |
| | | | | PDPK, |
| | | | | CDK5, |
| | | | | GSK3Beta; |
| | | | | S524/ |
| | | | | GSK3; |
| | | | | S526/; |
| NVKSKIGSTENLK | B154-E | MICROTUBULE- | N571 | S578/ |
| | | ASSOCIATED | K583 | MARK1, |
| | | PROTEIN TAU | | CaM Kinase |
| | | | | II, C- |
| | | | | Kinase, |
| | | | | GSK3, |
| | | | | PKA, PKC, |
| | | | | P110K, PK, |
| | | | | A-Kinase; |
| HQDQEGDTDAG | B154-N | MICROTUBULE- | H31 | T38/CK2 |
| LK | | ASSOCIATED | K43 | |
| | | PROTEIN TAU | | |
| ILVSTVKSKRREH | MIR1_HUMAN | MINIMUM | I67 H79 | |
| | | POTASSIUM ION | | |
| | | CHANNEL-RELATED | | |
| | | PEPTIDE 1 | | |
| | | (MIRP1) (MINK- | | |
| | | RELATED PEPTIDE | | |
| | | 1). | | |
| IVAILVSTVKSKR | MIR1_HUMAN | MINIMUM | I64 R76 | |
| | | POTASSIUM ION | | |
| | | CHANNEL-RELATED | | |
| | | PEPTIDE 1 (MIRP1) | | |
| | | (MINK-RELATED | | |
| | | PEPTIDE 1). | | |
| TSFMMTPYVVT | MK10_HUMAN | MITOGEN- | T216 | T221/ |
| RY | | ACTIVATED | Y228 | MAP2K4; |
| | | PROTEIN KINASE 10 | | Y223/ |
| | | (EC 2.7.1.—) (STRESS- | | MAP2K4 |
| | | ACTIVATED | | |
| | | PROTEIN KINASE | | |
| | | JNK3) (C-JUN N- | | |
| | | TERMINAL KINASE | | |
| | | 3) (MAP KINASE P49 | | |
| | | 3F12). | | |
| AGTSFMMTPYV | MK10_HUMAN | MITOGEN- | A214 | T221/ |
| VT | | ACTIVATED | T226 | MAP2K4; |
| | | PROTEIN KINASE 10 | | Y223/ |
| | | (EC 2.7.1.—) (STRESS- | | MAP2K4 |
| | | ACTIVATED | | |
| | | PROTEIN KINASE | | |
| | | JNK3) (C-JUN N- | | |
| | | TERMINAL KINASE | | |
| | | 3) (MAP KINASE P49 | | |
| | | 3F12). | | |
| ADSEMTGYVVT | MK12_HUMAN | MITOGEN- | A178 | T183/ |
| RW | | ACTIVATED | W190 | MAP2K3, |
| | | PROTEIN KINASE 12 | | MAP2K6; |
| | | (EC 2.7.1.—) | | Y185/ |
| | | (EXTRACELLULAR | | MAP2K3, |
| | | SIGNAL- | | MAP2K4, |
| | | REGULATED KINASE | | MAP2K6 |
| | | 6) (EC | | |
| | | 2.7.1.—) (ERK6) (ERK5) | | |
| | | (STRESS- | | |
| | | ACTIVATED | | |
| | | PROTEIN | | |
| | | KINASE-3) | | |
| | | (MITOGEN- | | |
| | | ACTIVATED | | |
| | | PROTEIN KINASE P38 | | |
| | | GAM A) (MAP | | |
| | | KINASE P38 | | |
| | | GAMMA). | | |
| RHTDDEMTGYV | MK14_HUMAN | MITOGEN- | R173 | T180/ |
| AT | | ACTIVATED | T185 | MAP2K3, |
| | | PROTEIN KINASE 14 | | MAP2K6; |
| | | (EC 2.7.1.—) | | Y182/ |
| | | (MITOGEN- | | MAP2K3, |
| | | ACTIVATED | | MAP2K6 |
| | | PROTEIN KINASE | | |
| | | P38) (MAP KINASE | | |
| | | P38) (CYTOKINE | | |
| | | SUPPRESSIVE ANTI- | | |
| | | INFLAMMATORY | | |
| | | DRUG BINDING | | |
| | | PROTEIN) (CSAID | | |
| | | BINDING PROTEIN) | | |
| | | (CSBP | | |
| | | (MAX-INTERACTING | | |
| | | PROTEIN 2) (MAP | | |
| | | KINASE MXI2). | | |
| AEHQYFMTEYV | MK07_HUMAN | MITOGEN- | A211 | T218/; |
| AT | | ACTIVATED | T223 | Y220/ |
| | | PROTEIN KINASE 7 | | |
| | | (EC 2.7.1.—) | | |
| | | (EXTRACELLULAR | | |
| | | SIGNAL- | | |
| | | REGULATED KINASE | | |
| | | 5) (ERK5) (ERK4) | | |
| | | (BMK1 KINASE). | | |
| ATRGRGSSVGG | M3K5_HUMAN | MITOGEN- | A76 S88 | S83/AKT1 |
| GS | | ACTIVATED | | |
| | | PROTEIN KINASE | | |
| | | KINASE KINASE 5 | | |
| | | (EC 2.7.1.—) | | |
| | | (MAPK/ERK KINASE | | |
| | | KINASE 5) (MEK | | |
| | | KINASE 5) | | |
| | | (MEKK 5) | | |
| | | (APOPTOSIS SIGNAL- | | |
| | | REGULATING | | |
| | | KINASE 1) | | |
| | | (ASK-1). | | |
| PKLGRRHSMEN | LGN_HUMAN | MOSAIC PROTEIN | P394 | S401/PKA |
| ME | | LGN. | E406 | |
| DLLSRFQSNRM | LGN_HUMAN | MOSAIC PROTEIN | D487 | S494/PKC |
| DD | | LGN. | D499 | |
| AEKHLEISREVGD | LGN_HUMAN | MOSAIC PROTEIN | A338 | S345/PKG |
| | | LGN. | D350 | |
| CQRHLDISRELND | LGN_HUMAN | MOSAIC PROTEIN | C118 | S125/PKG |
| | | LGN. | D130 | |
| ILVKCQGSRLD | LGN_HUMAN | MOSAIC PROTEIN | I593 | S600/PKG |
| DQ | | LGN. | Q605 | |
| SGLYRSPSMPE | MPI3_HUMAN | M-PHASE INDUCER | S209 | S214/cdc2- |
| NL | | PHOSPHATASE 3 (EC | L221 | cyclin B |
| | | 3.1.3.48). | | kinase; |
| | | | | S216/Prk, |
| | | | | CHK1, |
| | | | | CHK2, |
| | | | | MAPK14, |
| | | | | Plk3, C- |
| | | | | TAK1 |
| PPDAADASPVV | ZWIA_HUMAN | M-PHASE | P71 A83 | S78 |
| AA | | PHOSPHOPROTEIN 5 | | |
| | | (FRAGMENT). | | |
| ESLSYAPSPLQKP | ZWIA_HUMAN | M-PHASE | E58 P70 | S65 |
| | | PHOSPHOPROTEIN 5 | | |
| | | (FRAGMENT). | | |
| SGFQVSETPRQAP | ZWIA_HUMAN | M-PHASE | S44 P56 | T51 |
| | | PHOSPHOPROTEIN 5 | | |
| | | (FRAGMENT). | | |
| GSGLLCVSPWP | ZWIA_HUMAN | M-PHASE | G85 | S92 |
| FV | | PHOSPHOPROTEIN 5 | V97 | |
| | | (FRAGMENT). | | |
| EDENGDITPIKAK | MPH6_HUMAN | M-PHASE | E140 | T147 |
| | | PHOSPHOPROTEIN 6. | K152 | |
| RGRRKKKTPRK | MPP8_HUMAN | M-PHASE | R100 | T107 |
| AE | | PHOSPHOPROTEIN 8 | E112 | |
| | | (FRAGMENT). | | |
| AFDLFKLTPEEKN | MPP8_HUMAN | M-PHASE | A220 | T227 |
| | | PHOSPHOPROTEIN 8 | N232 | |
| | | (FRAGMENT). | | |
| LMPVSAQTPKG | MPP8_HUMAN | M-PHASE | L151 | T158 |
| RR | | PHOSPHOPROTEIN 8 | R163 | |
| | | (FRAGMENT). | | |
| RSPKENLSPGFSH | MPP9_HUMAN | M-PHASE | R21 | S22; S 28 |
| | | PHOSPHOPROTEIN 9 | H33 | |
| | | (FRAGMENT). | | |
| LLSKNESSPIRFD | MPP9_HUMAN | M-PHASE | L34 D46 | S41 |
| | | PHOSPHOPROTEIN 9 | | |
| | | (FRAGMENT). | | |
| TPVTVAYSPKRSP | MPP9_HUMAN | M-PHASE | T11 P23 | T11; S18; |
| | | PHOSPHOPROTEIN 9 | | S22 |
| | | (FRAGMENT). | | |
| QCKPVSVTPQG | MPP9_HUMAN | M-PHASE | Q123 | T130 |
| ND | | PHOSPHOPROTEIN 9 | D135 | |
| | | (FRAGMENT). | | |
| VAYSPKRSPKE | MPP9_HUMAN | M-PHASE | V15 L27 | S18; S22 |
| NL | | PHOSPHOPROTEIN 9 | | |
| | | (FRAGMENT). | | |
| TKREIMLTPVTVA | MPP9_HUMAN | M-PHASE | T4 A16 | T11 |
| | | PHOSPHOPROTEIN 9 | | |
| | | (FRAGMENT). | | |
| AALSRMPSPGG | MPP9_HUMAN | M-PHASE | A164 | S171 |
| RI | | PHOSPHOPROTEIN 9 | I176 | |
| | | (FRAGMENT). | | |
| SSNDSRSSLIRKR | B037-A | MULTIDRUG | S654 | S661/PKC |
| | | RESISTANCE | R666 | |
| | | PROTEIN 1 (P- | | |
| | | GLYCOPROTEIN | | |
| | | 1) | | |
| KIPKRPGSVHRTP | ACM1_HUMAN | MUSCARINIC | K444 | S451; T455 |
| | | ACETYLCHOLINE | P456 | |
| | | RECEPTOR M1. | | |
| CNKAFRDTFRL | ACM1_HUMAN | MUSCARINIC | C421 | T428 |
| LL | | ACETYLCHOLINE | L433 | |
| | | RECEPTOR M1. | | |
| VQGEEKESSND | B353-F | MUSCARINIC | V275 | S282/ |
| ST | | ACETYLCHOLINE | T287 | GRK; S283/ |
| | | RECEPTOR M2 | | GRK; |
| | | | | S286/ |
| | | | | GRK; T287/ |
| | | | | GRK |
| VANQDPVSPSL | B353-A | MUSCARINIC | V225 | S232/ |
| VQ | | ACETYLCHOLINE | Q237 | GRK; S234/ |
| | | RECEPTOR M2 | | GRK |
| SNDSTSVSAVASN | B353-K | MUSCARINIC | S283 | S283/ |
| | | ACETYLCHOLINE | N295 | GRK; S286/ |
| | | RECEPTOR M2 | | GRK; |
| | | | | T287/ |
| | | | | GRK; S288/ |
| | | | | GRK; |
| | | | | S290/ |
| | | | | GRK; S294/ |
| | | | | GRK |
| KAPRDPVTENC | B353-E | MUSCARINIC | K264 | T271/GRK |
| VQ | | ACETYLCHOLINE | Q276 | |
| | | RECEPTOR M2 | | |
| TQDENTVSTSL | B353-O | MUSCARINIC | T302 | T302/ |
| GH | | ACETYLCHOLINE | H314 | GRK; T307/ |
| | | RECEPTOR M2 | | GRK; |
| | | | | S309/ |
| | | | | GRK; T310/ |
| | | | | GRK; |
| | | | | S311/GRK |
| EKESSNDSTSVSA | B353-H | MUSCARINIC | E279 | S282/ |
| | | ACETYLCHOLINE | A291 | GRK; S283/ |
| | | RECEPTOR M2 | | GRK; |
| | | | | S286/ |
| | | | | GRK; T287/ |
| | | | | GRK; |
| | | | | S288/ |
| | | | | GRK; S290/ |
| | | | | GRK |
| EITQDENTVSTSL | B353-N | MUSCARINIC | E300 | T302/ |
| | | ACETYLCHOLINE | L312 | GRK; T307/ |
| | | RECEPTOR M2 | | GRK; |
| | | | | S309/ |
| | | | | GRK; T310/ |
| | | | | GRK; |
| | | | | S311/GRK |
| TSVSAVASNMR | B353-L | MUSCARINIC | T287 | T287/ |
| DD | | ACETYLCHOLINE | D299 | GRK; S288/ |
| | | RECEPTOR M2 | | GRK; |
| | | | | S290/ |
| | | | | GRK; S294/ |
| | | | | GRK |
| NMRDDEITQDE | B353-M | MUSCARINIC | N295 | T302/ |
| NT | | ACETYLCHOLINE | T307 | GRK; T307/ |
| | | RECEPTOR M2 | | GRK |
| CNATFKKTFRH | ACM4_HUMAN | MUSCARINIC | C456 | T463 |
| LL | | ACETYLCHOLINE | L468 | |
| | | RECEPTOR M4. | | |
| CNRTFRKTFKM | ACM5_HUMAN | MUSCARINIC | C498 | T501; T505 |
| LL | | ACETYLCHOLINE | L510 | |
| | | RECEPTOR M5. | | |
| CYALCNRTFRK | ACM5_HUMAN | MUSCARINIC | C494 | T501; T505 |
| TF | | ACETYLCHOLINE | F506 | |
| | | RECEPTOR M5. | | |
| RKPGLRRSPIKKV | MYBB_HUMAN | MYB-RELATED | R570 | S577/ |
| | | PROTEIN B (B-MYB). | V582 | Cyclin A |
| | | | | CDK2 |
| LDSCNSLTPKSTP | MYBB_HUMAN | MYB-RELATED | L433 | T440/ |
| | | PROTEIN B (B-MYB). | P445 | Cyclin A |
| | | | | CDK2; |
| | | | | T444/ |
| | | | | Cyclin A |
| | | | | CDK2 |
| TPLHRDKTPLH | MYBB_HUMAN | MYB-RELATED | T487 | T487/ |
| QK | | PROTEIN B (B-MYB). | K499 | Cyclin A |
| | | | | CDK2; |
| | | | | T494/ |
| | | | | Cyclin A |
| | | | | CDK2 |
| NSLTPKSTPVKTL | MYBB_HUMAN | MYB-RELATED | N437 | T440/ |
| | | PROTEIN B (B-MYB). | L449 | Cyclin A |
| | | | | CDK2; |
| | | | | T444/ |
| | | | | Cyclin A |
| | | | | CDK2 |
| SQKVVVTTPLH | MYBB_HUMAN | MYB-RELATED | S480 | T487/ |
| RD | | PROTEIN B (B-MYB). | D492 | Cyclin A |
| | | | | CDK2 |
| DNTPHTPTPFKNA | MYBB_HUMAN | MYB-RELATED | D513 | T515/ |
| | | PROTEIN B (B-MYB). | A525 | CKK2; |
| | | | | T518/ |
| | | | | CDK2; |
| | | | | T520/ |
| | | | | Cyclin A |
| | | | | CDK2 |
| KKFELLPTPPLSP | MYC_HUMAN | MYC PROTO- | K51 P63 | T58/CK2, |
| | | ONCOGENE PROTEIN | | GSK; S62/ |
| | | (C-MYC). | | JNK1, ERT |
| LLPTPPLSPSRRS | MYC_HUMAN | MYC PROTO- | L55 S67 | T58/CK2, |
| | | ONCOGENE PROTEIN | | GSK; S62/ |
| | | (C-MYC). | | JNK1, ERT |
| MPLNVSFTNRN | MYC_HUMAN | MYC PROTO- | M1 D13 | T8/c-RAF |
| YD | | ONCOGENE PROTEIN | | |
| | | (C-MYC). | | |
| TPRTPPPSQGKGR | MBP_HUMAN | MYELIN BASIC | T229 | T232 |
| | | PROTEIN (MBP). | R241 | |
| GLSLSRFSWGA | MBP_HUMAN | MYELIN BASIC | G242 | S249 |
| EG | | PROTEIN (MBP). | G254 | |
| PLPSHARSQPGLC | MBP_HUMAN | MYELIN BASIC | P68 C80 | |
| | | PROTEIN (MBP). | | |
| QGKGRGLSLSR | MBP_HUMAN | MYELIN BASIC | Q237 | S249 |
| FS | | PROTEIN (MBP). | S249 | |
| FKLGGRDSRSG | MBP_HUMAN | MYELIN BASIC | F288 | S295; S299 |
| SP | | PROTEIN (MBP). | P300 | |
| PGRSPLPSHARSQ | MBP_HUMAN | MYELIN BASIC | P64 Q76 | |
| | | PROTEIN (MBP). | | |
| KGRGLSLSRFS | MBP_HUMAN | MYELIN BASIC | K239 | S249 |
| WG | | PROTEIN (MBP). | G251 | |
| SKYLATASTMD | MBP_HUMAN | MYELIN BASIC | S146 | |
| HA | | PROTEIN (MBP). | A158 | |
| FLPRHRDTGILDS | MBP_HUMAN | MYELIN BASIC | F162 | |
| | | PROTEIN (MBP). | S174 | |
| RPSQRHGSKYL | MBP_HUMAN | MYELIN BASIC | R139 | S141 |
| AT | | PROTEIN (MBP). | T151 | |
| LCNMYKDSHHP | MBP_HUMAN | MYELIN BASIC | L79 R91 | |
| AR | | PROTEIN (MBP). | | |
| GRASDYKSAHK | MBP_HUMAN | MYELIN BASIC | G263 | |
| GF | | PROTEIN (MBP). | F275 | |
| PWLKPGRSPLPSH | MBP_HUMAN | MYELIN BASIC | P60 H72 | |
| | | PROTEIN (MBP). | | |
| VDAQGTLSKIFKL | MBP_HUMAN | MYELIN BASIC | V278 | |
| | | PROTEIN (MBP). | L290 | |
| ARTAHYGSLPQ | MBP_HUMAN | MYELIN BASIC | A198 | |
| KS | | PROTEIN (MBP). | S210 | |
| PQKSHGRTQDE | MBP_HUMAN | MYELIN BASIC | P207 | |
| NP | | PROTEIN (MBP). | P219 | |
| HFFKNIVTPRTPP | MBP_HUMAN | MYELIN BASIC | H222 | T232 |
| | | PROTEIN (MBP). | P234 | |
| FGYGGRASDYK | MBP_HUMAN | MYELIN BASIC | F259 | |
| SA | | PROTEIN (MBP). | A271 | |
| KGVDAQGTLSK | MBP_HUMAN | MYELIN BASIC | K276 | |
| IF | | PROTEIN (MBP). | F288 | |
| YGSLPQKSHGR | MBP_HUMAN | MYELIN BASIC | Y203 | |
| TQ | | PROTEIN (MBP). | Q215 | |
| HGSKYLATAST | MBP_HUMAN | MYELIN BASIC | H144 | |
| MD | | PROTEIN (MBP). | D156 | |
| KNIVTPRTPPPSQ | MBP_HUMAN | MYELIN BASIC | K225 | T232 |
| | | PROTEIN (MBP). | Q237 | |
| RDTGILDSIGRFF | MBP_HUMAN | MYELIN BASIC | R167 | |
| | | PROTEIN (MBP). | F179 | |
| KGMMPPLSEEE | MEFA_HUMAN | MYOCYTE-SPECIFIC | K282 | S289/CK2; |
| EL | | ENHANCER FACTOR | L294 | |
| | | 2A (SERUM | | |
| | | RESPONSE FACTOR- | | |
| | | LIKE PROTEIN 1). | | |
| RKGAGDGSDEE | B073-B | MYOSIN HEAVY | R1937 | S1944/ |
| VD | | CHAIN, NONMUSCLE | D1949 | PKC, CK2 |
| | | TYPE A (NMMHC-A) | | |
| AMNREVSSLKN | B073-A | MYOSIN HEAVY | A1910 | S1917/ |
| KL | | CHAIN, NONMUSCLE | L1922 | PKC, CK2 |
| | | TYPE A (NMMHC-A) | | |
| KKRPQRATSNV | MLRM_HUMAN | MYOSIN | K10 | T17/ |
| FA | | REGULATORY | A22 | MLCK; |
| | | LIGHT CHAIN 2, | | S18/ |
| | | NONSARCOMERIC | | MLCK |
| | | (MYOSIN RLC). | | |
| DKKGNFNYVEF | A055-D | MYOSIN | D148 | Y155/ |
| TR | | REGULATORY | R160 | EGFR |
| | | LIGHT CHAIN 2, | | |
| | | SMOOTH MUSCLE | | |
| | | ISOFORM | | |
| AGGGRRISDSHED | MYPC_HUMAN | MYOSIN-BINDING | A277 | S284/ |
| | | PROTEIN C, | D289 | PKA, PKC |
| | | CARDIAC-TYPE | | |
| | | (CARDIAC MYBP-C) | | |
| | | (C-PROTEIN, | | |
| | | CARDIAC MUSCLE | | |
| | | ISOFORM). | | |
| LSAFRRTSLAGGG | MYPC_HUMAN | MYOSIN-BINDING | L268 | S275/ |
| | | PROTEIN C, | G280 | PKA, PKC |
| | | CARDIAC-TYPE | | |
| | | (CARDIAC MYBP-C) | | |
| | | (C-PROTEIN, | | |
| | | CARDIAC MUSCLE | | |
| | | ISOFORM). | | |
| SLLKKRDSFRTPR | MYPC_HUMAN | MYOSIN-BINDING | S297 | S304/ |
| | | PROTEIN C, | R309 | PKA, PKC |
| | | CARDIAC-TYPE | | |
| | | (CARDIAC MYBP-C) | | |
| | | (C-PROTEIN, | | |
| | | CARDIAC MUSCLE | | |
| | | ISOFORM). | | |
| KRFSFKKSFKLSG | MACS_HUMAN | MYRISTOYLATED | K155 | S158/PKC; |
| | | ALANINE-RICH C- | G167 | S162/PKC; |
| | | KINASE SUBSTRATE | | |
| | | (MARCKS) (PROTEIN | | S166/PKC |
| | | KINASE C | | |
| | | SUBSTRATE, 80 KDA | | |
| | | PROTEIN, LIGHT | | |
| | | CHAIN) (PKCSL) | | |
| | | (80K-L PROTEIN). | | |
| FKKSFKLSGFSFK | MACS_HUMAN | MYRISTOYLATED | F159 | S162/PKC; |
| | | ALANINE-RICH C- | K171 | S166/PKC; |
| | | KINASE SUBSTRATE | | S169/PKC |
| | | (MARCKS) (PROTEIN | | |
| | | KINASE C | | |
| | | SUBSTRATE, 80 KDA | | |
| | | PROTEIN, LIGHT | | |
| | | CHAIN) (PKCSL) | | |
| | | (80K-L PROTEIN). | | |
| KKKKKRFSFKK | MACS_HUMAN | MYRISTOYLATED | K151 | S158/PKC; |
| SF | | ALANINE-RICH C- | F163 | S162/PKC |
| | | KINASE SUBSTRATE | | |
| | | (MARCKS) (PROTEIN | | |
| | | KINASE C | | |
| | | SUBSTRATE, 80 KDA | | |
| | | PROTEIN, LIGHT | | |
| | | CHAIN) (PKCSL) | | |
| | | (80K-L PROTEIN). | | |
| SSVIGWPTVRERM | NEF_HV1H2 | NEGATIVE FACTOR | S8 M20 | T15/PKC |
| | | (F-PROTEIN) (27 KDA | | |
| | | PROTEIN) (3′ORF). | | |
| GLVEVASYCEE | G45B_HUMAN | NEGATIVE | G134 | Y141/CK2 |
| SR | | GROWTH- | R146 | |
| | | REGULATORY | | |
| | | PROTEIN MYD118 | | |
| | | (MYELOID | | |
| | | DIFFERENTIATION | | |
| | | PRIMARY RESPONSE | | |
| | | PROTEIN MYD118) | | |
| | | (GROWTH ARREST | | |
| | | AND DNA-DAMAGE- | | |
| | | INDUCIBLE PROTEIN | | |
| | | GADD45 BETA). | | |
| PPTETGESSQAEE | NEUM_HUMAN | NEUROMODULIN | P195 | S202/CK2; |
| | | (AXONAL | E207 | S203/CK2 |
| | | MEMBRANE | | |
| | | PROTEIN GAP-43) | | |
| | | (PP46) (B-50) | | |
| | | (PROTEIN F1) | | |
| | | (CALMODULIN- | | |
| | | BINDING PROTEIN P- | | |
| | | 57). | | |
| AATKIQASFRG | NEUM_HUMAN | NEUROMODULIN | A34 I46 | S41/PKC |
| HI | | (AXONAL | | |
| | | MEMBRANE | | |
| | | PROTEIN GAP-43) | | |
| | | (PP46) (B-50) | | |
| | | (PROTEIN F1) | | |
| | | (CALMODULIN- | | |
| | | BINDING | | |
| | | PROTEIN P-57). | | |
| PGPQSPGSPLEEE | B060-G | NEUTROPHIL | P341 | S345/ |
| | | CYTOSOL FACTOR 2 | E353 | MAPK; |
| | | (P47-PHOX) | | S348/CK2, |
| | | | | MAPK |
| AHSIHQRSRKRLS | B060-C | NEUTROPHIL | A308 | S315/PKC |
| | | CYTOSOL FACTOR 2 | S320 | zeta, PKC |
| | | (P47-PHOX) | | alpha, PKC |
| | | | | Beta II, |
| | | | | PKC delta; |
| | | | | S320/PKC |
| | | | | alpha; PKC |
| | | | | Beta II, |
| | | | | PKC delta, |
| | | | | PKA |
| QARPGPQSPGSPL | B060-F | NEUTROPHIL | Q338 | S345/ |
| | | CYTOSOL FACTOR 2 | L350 | MAPK; |
| | | (P47-PHOX) | | S348/CK2, |
| | | | | MAPK |
| RGAPPRRSSIRNA | B060-A | NEUTROPHIL | R296 | S303/PKC |
| | | CYTOSOL FACTOR 2 | A308 | zeta, PKC |
| | | (P47-PHOX) | | alpha, PKC |
| | | | | Beta II, |
| | | | | PKC |
| | | | | delta; S304/ |
| | | | | PKC zeta, |
| | | | | PKC alpha, |
| | | | | PKC Beta |
| | | | | II, PKC |
| | | | | delta |
| DLILNRCSESTKR | B060-H | NEUTROPHIL | D372 | S379/PKC |
| | | CYTOSOL FACTOR 2 | R384 | alpha, PKC |
| | | (P47-PHOX) | | Beta II, |
| | | | | PKC delta |
| QRSRKRLSQDA | B060-D | NEUTROPHIL | Q313 | S315/PKC |
| YR | | CYTOSOL FACTOR 2 | R325 | zeta, PKC |
| | | (P47-PHOX) | | alpha, PKC |
| | | | | Beta II, |
| | | | | PKC |
| | | | | delta; S320/ |
| | | | | PKC alpha; |
| | | | | PKC Beta |
| | | | | II, PKC |
| | | | | delta, |
| | | | | PKA |
| QDAYRRNSVRF | B060-E | NEUTROPHIL | Q321 | S328/PKC |
| LQ | | CYTOSOL FACTOR 2 | Q333 | alpha, PKC |
| | | (P47-PHOX) | | Beta II, |
| | | | | PKC delta |
| CMDKYRLSCLE | NS2A_HUMAN | NITRIC OXIDE | C571 | S578/PKA |
| EE | | SYNTHASE, | E583 | |
| | | INDUCIBLE (EC | | |
| | | 1.14.13.39) (NOS, | | |
| | | TYPE II) (INDUCIBLE | | |
| | | NOS) (INOS) | | |
| | | (HEPATOCYTE NOS) | | |
| | | (HEP-NOS). | | |
| ICRHVRYSTNN | NS2A_HUMAN | NITRIC OXIDE | I227 | S234/PKA |
| GN | | SYNTHASE, | N239 | |
| | | INDUCIBLE (EC | | |
| | | 1.14.13.39) (NOS, | | |
| | | TYPE II) (INDUCIBLE | | |
| | | NOS) (INOS) | | |
| | | (HEPATOCYTE NOS) | | |
| | | (HEP-NOS). | | |
| EFPSLRVSAGFLL | NS2A_HUMAN | NITRIC OXIDE | E885 | S892/PKA |
| | | SYNTHASE, | L897 | |
| | | INDUCIBLE (EC | | |
| | | 1.14.13.39) (NOS, | | |
| | | TYPE II) (INDUCIBLE | | |
| | | NOS) (INOS) | | |
| | | (HEPATOCYTE NOS) | | |
| | | (HEP-NOS). | | |
| TSGEDTLSDSDDE | MYCN_HUMAN | N-MYC PROTO- | T254 | S261/CK2; |
| | | ONCOGENE | E266 | S263/CK2 |
| | | PROTEIN. | | |
| SGEDTLSDSDDED | MYCN_HUMAN | N-MYC PROTO- | S255 | S261/CK2; |
| | | ONCOGENE | D267 | S263/CK2 |
| | | PROTEIN. | | |
| LHALGKATPIYLD | TRKC_HUMAN | NT-3 GROWTH | L824 | Y834/ |
| | | FACTOR RECEPTOR | D836 | autophosphorylation |
| | | (EC 2.7.1.112) (TRKC | | |
| | | TYROSINE KINASE) | | |
| | | (GP145-TRKC) (TRK- | | |
| | | C). | | |
| FGMSRDVYSTD | TRKC_HUMAN | NT-3 GROWTH | F698 | Y705/ |
| YY | | FACTOR RECEPTOR | Y710 | autophosphorylation; |
| | | (EC 2.7.1.112) (TRKC | | Y709/ |
| | | TYROSINE KINASE) | | autophosphorylation |
| | | (GP145-TRKC) (TRK- | | |
| | | C). | | |
| FVQLRRKSDLE | KBF1_HUMAN | NUCLEAR FACTOR | F330 | S337/PKA |
| TS | | NF-KAPPA-B P105 | S342 | |
| | | SUBUNIT (DNA- | | |
| | | BINDING FACTOR | | |
| | | KBF1) (EBP-1) | | |
| | | [CONTAINS: | | |
| | | NUCLEAR | | |
| | | FACTOR NF-KAPPA- | | |
| | | B P50 SUBUNIT]. | | |
| DEDSPSSPEDTSY | NRF1_HUMAN | NUCLEAR | D41 | S44/CK2; |
| | | RESPIRATORY | Y53 | S46/CK2; |
| | | FACTOR-1 (NRF-1) | | S47/CK2; |
| | | (ALPHA | | S52/CK2 |
| | | PALINDROMIC | | |
| | | BINDING PROTEIN) | | |
| | | (ALPHA-PAL). | | |
| DEEEDDDSEED | NUCL_HUMAN | NUCLEOLIN | D145 | S152 |
| EE | | (PROTEIN C23). | E157 | |
| KNAKKEDSDEE | NUCL_HUMAN | NUCLEOLIN | K137 | S144 |
| ED | | (PROTEIN C23). | D149 | |
| AAAAAPASEDE | NUCL_HUMAN | NUCLEOLIN | A176 | S183 |
| DD | | (PROTEIN C23). | D188 | |
| AVEEDAESEDE | NPM_HUMAN | NUCLEOPHOSMIN | A118 | S125/CK2 |
| EE | | (NPM) (NUCLEOLAR | E130 | |
| | | PHOSPHOPROTEIN | | |
| | | B23) (NUMATRIN) | | |
| | | (NUCLEOLAR | | |
| | | PROTEIN NO38). | | |
| PRSKGQESFKK | B159-B | NUCLEOPHOSMIN | P220 | S227/PKC |
| QE | | (NUCLEOLAR | E232 | |
| | | PHOSPHOPROTEIN | | |
| | | B23) | | |
| VLKEQTGSDDE | DCOR_HUMAN | ORNITHINE | V296 | S303/CK2 |
| DE | | DECARBOXYLASE | E308 | |
| | | (EC 4.1.1.17) (ODC). | | |
| KEVVRTDSLKG | NR41_HUMAN | ORPHAN NUCLEAR | K334 | S341/PKA |
| RR | | RECEPTOR HMR | R346 | |
| | | (EARLY RESPONSE | | |
| | | PROTEIN NAK1) (TR3 | | |
| | | ORPHAN | | |
| | | RECEPTOR). | | |
| GRRGRLPSKPK | NR41_HUMAN | ORPHAN NUCLEAR | G344 | S351/PKA |
| QP | | RECEPTOR HMR | P356 | |
| | | (EARLY RESPONSE | | |
| | | PROTEIN NAK1) (TR3 | | |
| | | ORPHAN | | |
| | | RECEPTOR). | | |
| YLSWGTASPYS | PMX1_HUMAN | PAIRED MESODERM | Y190 | S197 |
| AM | | HOMEOBOX | M202 | |
| | | PROTEIN 1 | | |
| | | (HOMEOBOX | | |
| | | PROTEIN PHOX1). | | |
| WTASSPYSTVPPY | PMX2_HUMAN | PAIRED MESODERM | W202 | |
| | | HOMEOBOX | Y214 | |
| | | PROTEIN 2 (PRX-2) | | |
| | | (FRAGMENT). | | |
| FLSEETPYSYPTG | A007-A | PAXILLIN | F24 G36 | Y31/FAK, |
| | | | | v-Src, Abl |
| YPTGNHTYQEI | A007-B | PAXILLIN | Y33 | Y40/Csk, |
| AV | | | V45 | v-Src, Abl |
| VGEEEHVYSFP | PAXI_HUMAN | PAXILLIN. | V111 | Y118/ |
| NK | | | K123 | FAK, v-Src, |
| | | | | Abl |
| SSTYQSTSETVSI | PEPA_HUMAN | PEPSIN A (EC | S123 | S130 |
| | | 3.4.23.1). | I135 | |
| QRSELDKSSAHSY | PERI_HUMAN | PERIPHERIN. | Q459 | Y471 |
| | | | Y471 | |
| PGLGRKLSDFG | PH4H_HUMAN | PHENYLALANINE-4- | P9 E21 | S16/PKA |
| QE | | HYDROXYLASE (EC | | |
| | | 1.14.16.1) (PAH) (PHE- | | |
| | | 4- | | |
| | | MONOOXYGENASE). | | |
| ERVSRKMSIQE | PHOS_HUMAN | PHOSDUCIN (PHD) | E66 E78 | S73/PKA |
| YE | | (33 KDA | | |
| | | PHOTOTRANSDUCING | | |
| | | PROTEIN) (MEKA | | |
| | | PROTEIN). | | |
| EEGTFRSSIRRLS | PLM_HUMAN | PHOSPHOLEMMAN. | E76 S88 | S83/PKA, |
| | | | | PKC; S88/ |
| | | | | PKA |
| SNVSPAISIHEIG | KPB1_HUMAN | PHOSPHORYLASE B | S978 | S985/PKA |
| | | KINASE ALPHA | G990 | |
| | | REGULATORY | | |
| | | CHAIN, SKELETAL | | |
| | | MUSCLE ISOFORM | | |
| | | (PHOSPHORYLASE | | |
| | | KINASE ALPHA M | | |
| | | SUBUNIT). | | |
| TGIMQLKSEIKQV | KPB1_HUMAN | PHOSPHORYLASE B | T1000 | S1007/ |
| | | KINASE ALPHA | V1012 | PKA |
| | | REGULATORY | | |
| | | CHAIN, SKELETAL | | |
| | | MUSCLE ISOFORM | | |
| | | (PHOSPHORYLASE | | |
| | | KINASE | | |
| | | ALPHA M SUBUNIT). | | |
| QVEFRRLSISAES | KPB1_HUMAN | PHOSPHORYLASE B | Q1011 | S1018/ |
| | | KINASE ALPHA | S1023 | PKA |
| | | REGULATORY | | |
| | | CHAIN, SKELETAL | | |
| | | MUSCLE ISOFORM | | |
| | | (PHOSPHORYLASE | | |
| | | KINASE | | |
| | | ALPHA M SUBUNIT). | | |
| KEFGVERSVRPTD | KPB1_HUMAN | PHOSPHORYLASE B | K965 | S972/PKA |
| | | KINASE ALPHA | D977 | |
| | | REGULATORY | | |
| | | CHAIN, SKELETAL | | |
| | | MUSCLE ISOFORM | | |
| | | (PHOSPHORYLASE | | |
| | | KINASE | | |
| | | ALPHA M SUBUNIT). | | |
| RLSISAESQSPGT | KPB1_HUMAN | PHOSPHORYLASE B | R1016 | S1018/ |
| | | KINASE ALPHA | T1028 | PKA |
| | | REGULATORY | | |
| | | CHAIN, SKELETAL | | |
| | | MUSCLE ISOFORM | | |
| | | (PHOSPHORYLASE | | |
| | | KINASE | | |
| | | ALPHA M SUBUNIT). | | |
| AGLTAEVSWKV | KPBB_HUMAN | PHOSPHORYLASE B | A4 E16 | S11/Auto |
| LE | | KINASE BETA | | |
| | | REGULATORY | | |
| | | CHAIN | | |
| | | (PHOSPHORYLASE | | |
| | | KINASE BETA | | |
| | | SUBUNIT). | | |
| SKVKRQSSTPSAP | KPBB_HUMAN | PHOSPHORYLASE B | S693 | S700/PKA |
| | | KINASE BETA | P705 | |
| | | REGULATORY | | |
| | | CHAIN | | |
| | | (PHOSPHORYLASE | | |
| | | KINASE BETA | | |
| | | SUBUNIT). | | |
| NENTEDQYSLV | A044-A | PI3-KINASE P85- | N600 | Y607/ |
| ED | | ALPHA SUBUNIT | D612 | Insulin |
| | | | | receptor; |
| | | | | S608/ |
| | | | | Phosphoinositide- |
| | | | | 3- |
| | | | | kinase, |
| | | | | catalytic |
| | | | | subunit, |
| | | | | gamma |
| HSWPWQVSLRT | PLMN_HUMAN | PLASMINOGEN (EC | H590 | S597 |
| RF | | 3.4.21.7) [CONTAINS: | F602 | |
| | | ANGIOSTATIN]. | | |
| KKDTETVYSEV | PEC1_HUMAN | PLATELET | K706 | Y713 |
| RK | | ENDOTHELIAL CELL | K718 | |
| | | ADHESION | | |
| | | MOLECULE (PECAM- | | |
| | | 1) (CD31 ANTIGEN) | | |
| | | (ENDOCAM) (GPIIA′). | | |
| ARAAARLSLTD | B103-A | PLATELET | A184 | S191/PKA |
| PL | | GLYCOPROTEIN IB | L196 | |
| | | BETA CHAIN (GP-IB | | |
| | | BETA) | | |
| GQKFARKSTRR | B189-A | PLECKSTRIN | G106 | S113/PKC; |
| SI | | (PLATELET P47 | I118 | T114/ |
| | | PROTEIN) | | PKC; S117/ |
| | | | | PKC |
| QAIKMDRYKDN | B189-C | PLECKSTRIN | Q921 | Y928/ |
| FT | | (PLATELET P47 | T933 | autophosphorylation |
| | | PROTEIN) | | |
| AGMEFSRSKSD | EDG1_HUMAN | PROBABLE G | A345 | S352 |
| NS | | PROTEIN-COUPLED | S357 | |
| | | RECEPTOR EDG-1. | | |
| QSKVPFRSRSPSE | GPR6_HUMAN | PROBABLE G | Q349 | S356; S358; |
| | | PROTEIN-COUPLED | E361 | S360 |
| | | RECEPTOR GPR6. | | |
| DSSESEESAGPLL | PRGR_HUMAN | PROGESTERONE | | |
| | | RECEPTOR (PR). | | |
| GPFPGSQTSDTLP | PRGR_HUMAN | PROGESTERONE | G34 P46 | T41/CK |
| | | RECEPTOR (PR). | | |
| EQRMKESSFYS | PRGR_HUMAN | PROGESTERONE | E786 | S793/CK |
| LC | | RECEPTOR (PR). | C798 | |
| LRPDSEASQSPQY | PRGR_HUMAN | PROGESTERONE | L545 | S552/CK |
| | | RECEPTOR (PR). | Y557 | |
| EVEEEDSSESEES | PRGR_HUMAN | PROGESTERONE | | |
| | | RECEPTOR (PR). | | |
| VASVMQEYTQS | PSA2_HUMAN | PROTEASOME | V113 | Y120 |
| GG | | SUBUNIT ALPHA | G125 | |
| | | TYPE 2 (EC 3.4.99.46) | | |
| | | (PROTEASOME | | |
| | | COMPONENT C3) | | |
| | | (MACROPAIN | | |
| | | SUBUNIT | | |
| | | C3) | | |
| | | (MULTICATALYTIC | | |
| | | ENDOPEPTIDASE | | |
| | | COMPLEX | | |
| | | SUBUNIT C3). | | |
| RLDGENIYIRHSN | A012-A | PROTEIN 4.1 (BAND | R620 | Y627/ |
| | | 4.1) (P4.1) | N632 | EGFR |
| KFEEAERSLKD | G19P_HUMAN | PROTEIN KINASE C | K375 | S382/PKC |
| ME | | SUBSTRATE, 80 KDA | E387 | |
| | | PROTEIN, HEAVY | | |
| | | CHAIN (PKCSH) (80K- | | |
| | | H PROTEIN). | | |
| YKPLYIPSNRVND | G19P_HUMAN | PROTEIN KINASE C | Y82 | S89/PKC |
| | | SUBSTRATE, 80 KDA | D94 | |
| | | PROTEIN, HEAVY | | |
| | | CHAIN (PKCSH) (80K- | | |
| | | H PROTEIN). | | |
| SLKDMEESIRNLE | G19P_HUMAN | PROTEIN KINASE C | S382 | S382/PKC; |
| | | SUBSTRATE, 80 KDA | E394 | S389/PKC |
| | | PROTEIN, HEAVY | | |
| | | CHAIN (PKCSH) (80K- | | |
| | | H PROTEIN). | | |
| ENFDKFFTRGQPV | KPCA_HUMAN | PROTEIN KINASE C, | E624 | T631/ |
| | | ALPHA TYPE (EC | V636 | autophosphorylation |
| | | 2.7.1.—) (PKC-ALPHA). | | |
| SNFDKEFTRQPVE | KPC1_HUMAN | PROTEIN KINASE C, | S628 | T635/ |
| | | BETA-I TYPE (EC | E640 | autophosphorylation |
| | | 2.7.1.—) (PKC-BETA-1). | | |
| TRQPVELTPTDKL | KPC1_HUMAN | PROTEIN KINASE C, | T635 | T635/ |
| | | BETA-I TYPE (EC | L647 | autophosphorylation; |
| | | 2.7.1.—) (PKC-BETA-1). | | T642 |
| PPSEGEESTVRFA | KPC2_HUMAN | PROTEIN KINASE C, | P9 A21 | S16/ |
| | | BETA-II TYPE (EC | | autophosphorylation; |
| | | 2.7.1.—) (PKC-BETA- | | T17/ |
| | | 2). | | autophosphorylation |
| TRHPPVLTPPDQE | KPC2_HUMAN | PROTEIN KINASE C, | T634 | |
| | | BETA-II TYPE (EC | E646 | |
| | | 2.7.1.—) (PKC-BETA- | | |
| | | 2). | | |
| PEEKTTNTVSKFD | KPC2_HUMAN | PROTEIN KINASE C, | P317 | T324/ |
| | | BETA-II TYPE (EC | D329 | autophosphorylation |
| | | 2.7.1.—) (PKC-BETA- | | |
| | | 2). | | |
| RAKISQGTKVPEE | KPC2_HUMAN | PROTEIN KINASE C, | R307 | T314/ |
| | | BETA-II TYPE (EC | E319 | autophosphorylation |
| | | 2.7.1.—) (PKC-BETA- | | |
| | | 2). | | |
| NNFDQDFTREE | KPCE_HUMAN | PROTEIN KINASE C, | N696 | T703/ |
| PV | | EPSILON TYPE (EC | V708 | autophosphorylation |
| | | 2.7.1.—) (NPKC- | | |
| | | EPSILON). | | |
| TREEPVLTLVDEA | KPCE_HUMAN | PROTEIN KINASE C, | T703 | T703/ |
| | | EPSILON TYPE (EC | A715 | autophosphorylation; |
| | | 2.7.1.—) (NPKC- | | T710/ |
| | | EPSILON). | | autophosphorylation |
| TRAAPALTPPDRL | KPCG_HUMAN | PROTEIN KINASE C, | T648 | T648/ |
| | | GAMMA TYPE (EC | L660 | autophosphorylation; |
| | | 2.7.1.—) (PKC- | | T655/ |
| | | GAMMA). | | autophosphorylation |
| QIRRRRPTPATLV | IPP1_HUMAN | PROTEIN | Q28 | T35/PKA |
| | | PHOSPHATASE | V40 | |
| | | INHIBITOR 1 (IPP-1) | | |
| | | (I-1). | | |
| GDDEDACSDTE | IPP2_HUMAN | PROTEIN | G79 T91 | S86/CK2 |
| AT | | PHOSPHATASE | | |
| | | INHIBITOR 2 (IPP-2). | | |
| YRIQEQESSGEED | IPP2_HUMAN | PROTEIN | Y113 | S120/CK2; |
| | | PHOSPHATASE | D125 | S121/CK2 |
| | | INHIBITOR 2 (IPP-2). | | |
| MKIDEPSTPYHSM | IPP2_HUMAN | PROTEIN | M65 | T72/GSK- |
| | | PHOSPHATASE | M77 | 3Beta, |
| | | INHIBITOR 2 (IPP-2). | | NCLK |
| RSRVVGGSLRG | PTN1_HUMAN | PROTEIN-TYROSINE | R371 | S378/PKC, |
| AQ | | PHOSPHATASE 1B | Q383 | CK2 |
| | | (EC 3.1.3.48) (PTP- | | |
| | | 1B). | | |
| LRGAQAASPAK | PTN1_HUMAN | PROTEIN-TYROSINE | L379 | S386/ |
| GE | | PHOSPHATASE 1B | E391 | p34cdc2:cyclin B |
| | | (EC 3.1.3.48) (PTP- | | |
| | | 1B). | | |
| REDSARVYENV | A011-B | PROTEIN-TYROSINE | R573 | S576/PKC |
| GL | | PHOSPHATASE 2C | L585 | (alpha, beta |
| | | (SH-PTP2). | | 1, beta 2 |
| | | | | and eta); |
| | | | | Y580/ |
| | | | | PDGFR |
| SKRKGHEYTNI | A011-A | PROTEIN-TYROSINE | S535 | Y542/ |
| KY | | PHOSPHATASE 2C | Y547 | PDGFR |
| | | (SH-PTP2). | | |
| KKLERNLSFEIKK | B195-B | PROTEIN-TYROSINE | K428 | S435/ |
| | | PHOSPHATASE G1 | K440 | PKA, PKC |
| | | (PTP-PEST) | | |
| FMRLRRLSTKY | B195-A | PROTEIN-TYROSINE | F32 T44 | S39/PKA, |
| RT | | PHOSPHATASE G1 | | PKC |
| | | (PTP-PEST) | | |
| EGEEDTEYMTP | CBL_HUMAN | PROTO-ONCOGENE | E693 | Y700/ |
| SS | | C-CBL. | S705 | EGFR, Fyn, |
| | | | | Yes, Syk, |
| | | | | IR kinase |
| GPPEPGPYAQPSV | A056-A | PROTO-ONCOGENE | G214 | Y221/ |
| | | C-CRK | V226 | IGF1 |
| | | | | receptor, c- |
| | | | | ABL, EGFR |
| FCKRRVESGEG | DBL_HUMAN | PROTO-ONCOGENE | F735 | S742 |
| SD | | DBL [CONTAINS: | D747 | |
| | | MCF2]. | | |
| RQEDGGVYSSS | FER_HUMAN | PROTO-ONCOGENE | R707 | Y714/ |
| GL | | TYROSINE-PROTEIN | L719 | autophosphorylation |
| | | KINASE FER (EC | | |
| | | 2.7.1.112) (P94-FER) | | |
| | | (C-FER). | | |
| REEADGVYAAS | FES_HUMAN | PROTO-ONCOGENE | R706 | Y713/ |
| GG | | TYROSINE-PROTEIN | G718 | autophosphorylation |
| | | KINASE FES/FPS (EC | | |
| | | 2.7.1.112) (C-FES). | | |
| FTATEPQYQPGEN | A045-B | PROTO-ONCOGENE | F523 | Y530/Csk |
| | | TYROSINE-PROTEIN | N535 | |
| | | KINASE Fyn | | |
| QCKDKEATKLT | FYN_HUMAN | PROTO-ONCOGENE | Q4 E16 | T11/PKC |
| EE | | TYROSINE-PROTEIN | | |
| | | KINASE FYN (EC | | |
| | | 2.7.1.112) (P59-FYN) | | |
| | | (SYN) (SLK). | | |
| RLIEDNEYTAREG | LCK_HUMAN | PROTO-ONCOGENE | R386 | Y393/ |
| | | TYROSINE-PROTEIN | G398 | autophosphorylation |
| | | KINASE LCK (EC | | |
| | | 2.7.1.112) (P56-LCK) | | |
| | | (LSK) (T CELL- | | |
| | | SPECIFIC PROTEIN- | | |
| | | TYROSINE KINASE). | | |
| TPSDSLIYDDGLS | A051-D | PROTO-ONCOGENE | T1022 | Y1029/ |
| | | TYROSINE-PROTEIN | S1034 | autophosphorylation |
| | | KINASE RECEPTOR | | |
| | | RET | | |
| SRKVGPGYLGS | A051-B | PROTO-ONCOGENE | S819 | Y826/ |
| GG | | TYROSINE-PROTEIN | G831 | autophosphorylation |
| | | KINASE RECEPTOR | | |
| | | RET | | |
| TWIENKLYGMS | A051-E | PROTO-ONCOGENE | T1055 | Y1062/ |
| DP | | TYROSINE-PROTEIN | P1067 | autophosphorylation |
| | | KINASE RECEPTOR | | |
| | | RET | | |
| AQAFPVSYSSSGA | A051-A | PROTO-ONCOGENE | A680 | Y687/ |
| | | TYROSINE-PROTEIN | A692 | autophosphorylation |
| | | KINASE RECEPTOR | | |
| | | RET | | |
| LIEDNEYTARQ | SRC_HUMAN | PROTO-ONCOGENE | L412 | Y418/ |
| GA | | TYROSINE-PROTEIN | A424 | autophosphorylation |
| | | KINASE SRC (EC | | |
| | | 2.7.1.112) (P60-SRC) | | |
| | | (C-SRC). | | |
| RRAASMDSSSK | AFX1_HUMAN | PUTATIVE FORK | R192 | S196/PKB |
| LL | | HEAD DOMAIN | L204 | |
| | | TRANSCRIPTION | | |
| | | FACTOR AFX1. | | |
| PRSSSNASSVSTR | AFX1_HUMAN | PUTATIVE FORK | P257 | S261/PKB |
| | | HEAD DOMAIN | R269 | |
| | | TRANSCRIPTION | | |
| | | FACTOR AFX1. | | |
| NRYGMGTSVER | ODPA_HUMAN | PYRUVATE | N225 | S232/PDK |
| AA | | DEHYDROGENASE | A237 | |
| | | E1 COMPONENT | | |
| | | ALPHA SUBUNIT, | | |
| | | SOMATIC FORM, | | |
| | | MITOCHONDRIAL | | |
| | | (EC 1.2.4.1) | | |
| | | (PDHE1-A TYPE I). | | |
| TYRYHGHSMSD | ODPT_HUMAN | PYRUVATE | T284 | S291 |
| PG | | DEHYDROGENASE | G296 | |
| | | E1 COMPONENT | | |
| | | ALPHA SUBUNIT, | | |
| | | TESTIS-SPECIFIC | | |
| | | FORM, | | |
| | | MITOCHONDRIAL | | |
| | | (EC | | |
| | | 1.2.4.1) (PDHE1-A | | |
| | | TYPE II). | | |
| SMSDPGVSYRT | ODPT_HUMAN | PYRUVATE | S291 | S291 S298 |
| RE | | DEHYDROGENASE | E303 | |
| | | E1 COMPONENT | | |
| | | ALPHA SUBUNIT, | | |
| | | TESTIS-SPECIFIC | | |
| | | FORM, | | |
| | | MITOCHONDRIAL | | |
| | | (EC | | |
| | | 1.2.4.1) (PDHE1-A | | |
| | | TYPE II). | | |
| KDGATMKTFCG | KRAC_HUMAN | RAC-ALPHA | K301 | T308/ |
| TP | | SERINE/THREONINE | P313 | PDPK1 |
| | | KINASE (EC 2.7.1.—) | | |
| | | (RAC-PK-ALPHA) | | |
| | | (PROTEIN KINASE B) | | |
| | | (PKB) (C-AKT). | | |
| SQRQRSTSTPNVH | B193-A | RAF PROTO- | S252 | S259/ |
| | | ONCOGENE | H264 | PKA, AKT1 |
| | | SEINE/THREONINE- | | |
| | | PROTEIN KINASE | | |
| | | (RAF-1) | | |
| PKINRSASEPSLH | B193-C | RAF PROTO- | P614 | S621/ |
| | | ONCOGENE | H626 | PKA, |
| | | SEINE/THREONINE- | | RAF1, |
| | | PROTEIN KINASE | | AMPK |
| | | (RAF-1) | | |
| VKSRWSGSQQV | B193-B | RAF PROTO- | V492 | S494; S499/ |
| EQ | | ONCOGENE | Q504 | PKC alpha |
| | | SEINE/THREONINE- | | |
| | | PROTEIN KINASE | | |
| | | (RAF-1) | | |
| RPRGQRDSSYY | KRAF_HUMAN | RAF PROTO- | R331 | S338/ |
| WE | | ONCOGENE | E343 | PAK1, |
| | | SERINE/THREONINE- | | PAK2, |
| | | PROTEIN KINASE | | PAK3; |
| | | (EC 2.7.1.—) (RAF-1) | | S339; |
| | | (C-RAF). | | Y340; Y341/ |
| | | | | c-Src |
| TVDGKEIYNTIRR | A006-A | Ras-GTPASE- | T453 | Y460/ |
| | | ACTIVATING | R465 | PDGFR, |
| | | PROTEIN (Ras-GAP) | | EGFR, Lck |
| KSNVKIQSTPVKQ | RB1A_HUMAN | RAS-RELATED | K187 | S194/cdc2 |
| | | PROTEIN RAB-1A | Q199 | |
| | | (YPT1-RELATED | | |
| | | PROTEIN). | | |
| AALRQLRSPRR | B343-A | RAS-RELATED | A192 | S199/cdc2 |
| TQ | | PROTEIN RAB-4A | Q204 | |
| PGKARKKSSCQ | B130-A | RAS-RELATED | P172 | S179/PKA |
| LL | | PROTEIN RAP-1B | L184 | |
| | | (GTP-BINDING | | |
| | | PROTEIN SMG P21B) | | |
| TCSPQPEYVNQPD | ERB2_HUMAN | RECEPTOR PROTEIN- | T1132 | Y1139/ |
| | | TYROSINE KINASE | D1144 | autophosphorylation |
| | | ERBB-2 (EC | | |
| | | 2.7.1.112) | | |
| | | (P185ERBB2) (NEU | | |
| | | PROTO-ONCOGENE) | | |
| | | (C-ERBB- | | |
| | | 2) (TYROSINE | | |
| | | KINASE-TYPE CELL | | |
| | | SURFACE RECEPTOR | | |
| | | HER2) (MLN 19). | | |
| RNLYSGDYYRI | DDR2_HUMAN | RECEPTOR PROTEIN- | R733 | Y740/ |
| QG | | TYROSINE KINASE | G745 | autophosphorylation |
| | | TKT (EC | | |
| | | 2.7.1.112) (TYROSINE- | | |
| | | PROTEIN KINASE | | |
| | | TYRO 10) | | |
| | | (NEUROTROPHIC | | |
| | | TYROSINE KINASE, | | |
| | | RECEPTOR- | | |
| | | RELATED | | |
| | | 3). | | |
| SKSKDVLSAAE | RGS1_HUMAN | REGULATOR OF G- | S55 | S62/CK2 |
| VM | | PROTEIN SIGNALING | M67 | |
| | | 1 (RGS1) (EARLY | | |
| | | RESPONSE PROTEIN | | |
| | | 1R20) (B-CELL | | |
| | | ACTIVATION | | |
| | | PROTEIN | | |
| | | BL34). | | |
| ELKGTTHSLLD | RGS1_HUMAN | REGULATOR OF G- | E12 K24 | S19/CK2 |
| DK | | PROTEIN SIGNALING | | |
| | | 1 (RGS1) (EARLY | | |
| | | RESPONSE PROTEIN | | |
| | | 1R20) (B-CELL | | |
| | | ACTIVATION | | |
| | | PROTEIN | | |
| | | BL34). | | |
| HLESGMKSSKS | RGS1_HUMAN | REGULATOR OF G- | H47 | S54/PKC |
| KD | | PROTEIN SIGNALING | D59 | |
| | | 1 (RGS1) (EARLY | | |
| | | RESPONSE PROTEIN | | |
| | | 1R20) (B-CELL | | |
| | | ACTIVATION | | |
| | | PROTEIN | | |
| | | BL34). | | |
| EAQKVIYTLME | RGS1_HUMAN | REGULATOR OF G- | E160 | T167/CK2 |
| KD | | PROTEIN SIGNALING | D172 | |
| | | 1 (RGS1) (EARLY | | |
| | | RESPONSE PROTEIN | | |
| | | 1R20) (B-CELL | | |
| | | ACTIVATION | | |
| | | PROTEIN | | |
| | | BL34). | | |
| AKALGKRTAKY | NPT2_HUMAN | RENAL SODIUM- | A501 | T508/PKC |
| RW | | DEPENDENT | W513 | |
| | | PHOSPHATE | | |
| | | TRANSPORT | | |
| | | PROTEIN 2 | | |
| | | (SODIUM/PHOSPHATE | | |
| | | COTRANSPORTER | | |
| | | 2) | | |
| | | (NA(+)/PI | | |
| | | COTRANSPORTER 2) | | |
| | | (RENAL SODIUM- | | |
| | | PHOSPHATE | | |
| | | TRANSPORT | | |
| | | PROTEIN 2) (RENAL | | |
| | | NA+- | | |
| | | DEPENDENT | | |
| | | PHOSPHATE | | |
| | | COTRAN PORTER 2). | | |
| VNVIPPHTPVRTV | RB_HUMAN | RETINOBLASTOMA- | V366 | T373/ |
| | | ASSOCIATED | V378 | p34cdc2 |
| | | PROTEIN (PP110) | | |
| | | (P105-RB) (RB). | | |
| IYISPLKSPYKIS | RB_HUMAN | RETINOBLASTOMA- | I804 | S807/ |
| | | ASSOCIATED | S816 | p34cdc2; |
| | | PROTEIN (PP110) | | S811/ |
| | | (P105-RB) (RB). | | p34cdc2 |
| PINGSPRTPRRGQ | RB_HUMAN | RETINOBLASTOMA- | P245 | S249/ |
| | | ASSOCIATED | Q257 | p34cdc2; |
| | | PROTEIN (PP110) | | T252/ |
| | | (P105-RB) (RB). | | p34cdc2 |
| AVIPINGSPRTPR | RB_HUMAN | RETINOBLASTOMA- | A242 | S249/ |
| | | ASSOCIATED | R254 | p34cdc2; |
| | | PROTEIN (PP110) | | T252/ |
| | | (P105-RB) (RB). | | p34cdc2 |
| DRTSRDSSPVM | RBL2_HUMAN | RETINOBLASTOMA- | D959 | S962/ |
| RS | | LIKE PROTEIN 2 (130 KDA | S971 | CDK; S966/ |
| | | RETINOBLASTOMA- | | Cdk4; |
| | | ASSOCIATED | | S971/cdk4 |
| | | PROTEIN) (PRB2) | | |
| | | (P130) | | |
| | | (RBR-2). | | |
| SKALRISTPLTGV | RBL2_HUMAN | RETINOBLASTOMA- | S394 | T401/ |
| | | LIKE PROTEIN 2 (130 KDA | V406 | CDK4 |
| | | RETINOBLASTOMA- | | |
| | | ASSOCIATED | | |
| | | PROTEIN) (PRB2) | | |
| | | (P130) | | |
| | | (RBR-2). | | |
| RKSVPTVSKGT | RBL2_HUMAN | RETINOBLASTOMA- | R96 | |
| VE | | LIKE PROTEIN 2 (130 KDA | E108 | |
| | | RETINOBLASTOMA- | | |
| | | ASSOCIATED | | |
| | | PROTEIN) (PRB2) | | |
| | | (P130) (RBR-2). | | |
| VRYIKENSPCVTP | RBL2_HUMAN | RETINOBLASTOMA- | V406 | S413/ |
| | | LIKE PROTEIN 2 (130 KDA | P418 | CDK; T417/ |
| | | RETINOBLASTOMA- | | CDK |
| | | ASSOCIATED | | |
| | | PROTEIN) (PRB2) | | |
| | | (P130) | | |
| | | (RBR-2). | | |
| SPVMRSSSTLPVP | RBL2_HUMAN | RETINOBLASTOMA- | S966 | S966/cdk4; |
| | | LIKE PROTEIN 2 (130 KDA | P978 | S971/cdk4; |
| | | RETINOBLASTOMA- | | S972/cdk4; |
| | | ASSOCIATED | | S973/cdk4; |
| | | PROTEIN) (PRB2) | | T974/cdk4 |
| | | (P130) (RBR-2). | | |
| CIAGSPLTPRRVT | RBL2_HUMAN | RETINOBLASTOMA- | C635 | S639/cdk4; |
| | | LIKE PROTEIN 2 (130 KDA | T647 | T642/CDK |
| | | RETINOBLASTOMA- | | |
| | | ASSOCIATED | | |
| | | PROTEIN) (PRB2) | | |
| | | (P130) | | |
| | | (RBR-2). | | |
| VPTVSKGTVEG | RBL2_HUMAN | RETINOBLASTOMA- | V99 | |
| NY | | LIKE PROTEIN 2 (130 KDA | Y111 | |
| | | RETINOBLASTOMA- | | |
| | | ASSOCIATED | | |
| | | PROTEIN) (PRB2) | | |
| | | (P130) (RBR-2). | | |
| TLYDRYSSPPAST | RBL2_HUMAN | RETINOBLASTOMA- | T665 | S672/ |
| | | LIKE PROTEIN 2 (130 KDA | T677 | CDK4 |
| | | RETINOBLASTOMA- | | |
| | | ASSOCIATED | | |
| | | PROTEIN) (PRB2) | | |
| | | (P130) | | |
| | | (RBR-2). | | |
| LPVPQPSSAPPTP | RBL2_HUMAN | RETINOBLASTOMA- | L975 | S981/cdk4; |
| | | LIKE PROTEIN 2 (130 KDA | P987 | S982/cdk4; |
| | | RETINOBLASTOMA- | | T986/cdk4 |
| | | ASSOCIATED | | |
| | | PROTEIN) (PRB2) | | |
| | | (P130) | | |
| | | (RBR-2). | | |
| DEICIAGSPLTPR | RBL2_HUMAN | RETINOBLASTOMA- | D632 | S639/cdk4; |
| | | LIKE PROTEIN 2 (130 KDA | R644 | T642/CDK |
| | | RETINOBLASTOMA- | | |
| | | ASSOCIATED | | |
| | | PROTEIN) (PRB2) | | |
| | | (P130) | | |
| | | (RBR-2). | | |
| KGTVEGNYVSL | RBL2_HUMAN | RETINOBLASTOMA- | K104 | |
| TR | | LIKE PROTEIN 2 (130 KDA | R116 | |
| | | RETINOBLASTOMA- | | |
| | | ASSOCIATED | | |
| | | PROTEIN) (PRB2) | | |
| | | (P130) | | |
| | | (RBR-2). | | |
| DSPSDGGTPGR | RBL2_HUMAN | RETINOBLASTOMA- | D687 | S688/ |
| MP | | LIKE PROTEIN 2 (130 KDA | P699 | CDK; T694/ |
| | | RETINOBLASTOMA- | | CDK |
| | | ASSOCIATED | | |
| | | PROTEIN) (PRB2) | | |
| | | (P130) | | |
| | | (RBR-2). | | |
| SGSSDSRSHQNSP | RBL2_HUMAN | RETINOBLASTOMA- | S941 | S948/cdk4; |
| | | LIKE PROTEIN 2 (130 KDA | P953 | S952/cdk4 |
| | | RETINOBLASTOMA- | | |
| | | ASSOCIATED | | |
| | | PROTEIN) (PRB2) | | |
| | | (P130) | | |
| | | (RBR-2). | | |
| RLFVENDSPSDGG | RBL2_HUMAN | RETINOBLASTOMA- | R681 | S688/CDK |
| | | LIKE PROTEIN 2 (130 KDA | G693 | |
| | | RETINOBLASTOMA- | | |
| | | ASSOCIATED | | |
| | | PROTEIN) (PRB2) | | |
| | | (P130) (RBR-2). | | |
| GLGRSITSPTTLY | RBL2_HUMAN | RETINOBLASTOMA- | G655 | S662/CDK |
| | | LIKE PROTEIN 2 (130 KDA | Y667 | |
| | | RETINOBLASTOMA- | | |
| | | ASSOCIATED | | |
| | | PROTEIN) (PRB2) | | |
| | | (P130) (RBR-2). | | |
| KENSPCVTPVSTA | RBL2_HUMAN | RETINOBLASTOMA- | K410 | S413/ |
| | | LIKE PROTEIN 2 (130 KDA | A422 | CDK; T417/ |
| | | RETINOBLASTOMA- | | CDK |
| | | ASSOCIATED | | |
| | | PROTEIN) (PRB2) | | |
| | | (P130) | | |
| | | (RBR-2). | | |
| ELNKDRTSRDSSP | RBL2_HUMAN | RETINOBLASTOMA- | E955 | S962/ |
| | | LIKE PROTEIN 2 (130 KDA | P967 | CDK; S966/ |
| | | RETINOBLASTOMA- | | Cdk4 |
| | | ASSOCIATED | | |
| | | PROTEIN) (PRB2) | | |
| | | (P130) (RBR-2). | | |
| DSRSHQNSPTELN | RBL2_HUMAN | RETINOBLASTOMA- | D945 | S948/cdk4; |
| | | LIKE PROTEIN 2 (130 KDA | N957 | S952/cdk4 |
| | | RETINOBLASTOMA- | | |
| | | ASSOCIATED | | |
| | | PROTEIN) (PRB2) | | |
| | | (P130) | | |
| | | (RBR-2). | | |
| AFIAARGSFDGSS | RK_HUMAN | RHODOPSIN KINASE | A14 S26 | S21/ |
| | | (EC 2.7.1.125) (RK). | | autophosphorylation |
| IQDVGAFSTVK | RK_HUMAN | RHODOPSIN KINASE | I484 | S491/ |
| GV | | (EC 2.7.1.125) (RK). | V496 | autophosphorylation; |
| | | | | T492/ |
| | | | | autophosphorylation |
| ASATVSKTETSQV | OPSD_HUMAN | RHODOPSIN. | A333 | S334/RK; |
| | | | V345 | S338/RK; |
| | | | | T340 |
| RQTPVDSPDDSTL | K6B1_HUMAN | RIBOSOMAL | R365 | T367; S371/ |
| | | PROTEIN S6 KINASE | L377 | mTOR |
| | | (EC 2.7.1.—) (S6K) (P70- | | |
| | | S6K) | | |
| DRIDEKLSEILGM | RRPP_HRSVL | RNA POLYMERASE | D136 | S143 |
| | | ALPHA SUBUNIT (EC | M148 | |
| | | 2.7.7.48) | | |
| | | (PHOSPHOPROTEIN | | |
| | | P). | | |
| FDNNEEESSYSYE | RRPP_HRSVL | RNA POLYMERASE | F109 | S116; S117; |
| | | ALPHA SUBUNIT (EC | E121 | S119 |
| | | 2.7.7.48) | | |
| | | (PHOSPHOPROTEIN | | |
| | | P). | | |
| NEEESSYSYEEIN | RRPP_HRSVL | RNA POLYMERASE | N112 | S116; S117; |
| | | ALPHA SUBUNIT (EC | N124 | S119 |
| | | 2.7.7.48) | | |
| | | (PHOSPHOPROTEIN | | |
| | | P). | | |
| LHTLVVASAGP | RRPP_HRSVL | RNA POLYMERASE | L149 | S156; S161 |
| TS | | ALPHA SUBUNIT (EC | S161 | |
| | | 2.7.7.48) | | |
| | | (PHOSPHOPROTEIN | | |
| | | P). | | |
| VRRLRRLTARE | RYR1_HUMAN | RYANODINE | V4317 | T4324 |
| AA | | RECEPTOR, | A4329 | |
| | | SKELETAL MUSCLE | | |
| | | (SKELETAL MUSCLE | | |
| | | CALCIUM RELEASE | | |
| | | CHANNEL). | | |
| EQGKRNFSKAM | RYR1_HUMAN | RYANODINE | E3944 | S3951 |
| SV | | RECEPTOR, | V3956 | |
| | | SKELETAL MUSCLE | | |
| | | (SKELETAL MUSCLE | | |
| | | CALCIUM RELEASE | | |
| | | CHANNEL). | | |
| KKKTAKISQSA | RYR1_HUMAN | RYANODINE | K2836 | S2843/ |
| QT | | RECEPTOR, | T2848 | PKA, PKG |
| | | SKELETAL MUSCLE | | |
| | | (SKELETAL MUSCLE | | |
| | | CALCIUM RELEASE | | |
| | | CHANNEL). | | |
| VISDGGDSEQFID | PRPC_HUMAN | SALIVARY ACIDIC | V31 | S38 |
| | | PROLINE-RICH | D43 | |
| | | PHOSPHOPROTEIN | | |
| | | 1/2 (PRP-1/PRP-3) | | |
| | | (PRP-2/PRP-4) (PIF- | | |
| | | F/PIF-S) (PROTEIN | | |
| | | A/PROTEIN C) | | |
| | | [CONTAINS: PEPTIDE | | |
| | | P-C]. | | |
| ELILKPPSPISEA | STN2_HUMAN | SCG10 PROTEIN | E55 A67 | S62 |
| | | (SUPERIOR | | |
| | | CERVICAL | | |
| | | GANGLION-10 | | |
| | | PROTEIN), | | |
| | | STATHMIN 2. | | |
| AAGERRKSQEA | STN2_HUMAN | SCG10 PROTEIN | A90 | S97 |
| QV | | (SUPERIOR | V102 | |
| | | CERVICAL | | |
| | | GANGLION-10 | | |
| | | PROTEIN), | | |
| | | STATHMIN 2. | | |
| KEKMKELSMLS | STN2_HUMAN | SCG10 PROTEIN | K9 I21 | S16 |
| LI | | (SUPERIOR | | |
| | | CERVICAL | | |
| | | GANGLION-10 | | |
| | | PROTEIN), | | |
| | | STATHMIN 2. | | |
| EPHVTRRTPDYFL | P2AB_HUMAN | SERINE/THREONINE | E297 | T304 Y307 |
| | | PROTEIN | L309 | |
| | | PHOSPHATASE 2A, | | |
| | | CATALYTIC | | |
| | | SUBUNIT, BETA | | |
| | | ISOFORM (EC | | |
| | | 3.1.3.16) (PP2A- | | |
| | | BETA). | | |
| EGNNANYTEYV | STK9_HUMAN | SERINE/THREONINE- | E162 | T169 Y171 |
| AT | | PROTEIN KINASE 9 | T174 | |
| | | (EC 2.7.1.—). | | |
| ETSLMRTLCGTPT | CHK2_HUMAN | SERINE/THREONINE- | E377 | T383/ |
| | | PROTEIN KINASE | T389 | Chk2; T387/ |
| | | CHK2 (EC 2.7.1.—) | | Chk2 |
| | | (CDS1) | | |
| FAKTFVGTPYY | NEK2_HUMAN | SERINE/THREONINE- | F172 | T175/ |
| MS | | PROTEIN KINASE | S184 | autophosphorylation |
| | | NEK2 (EC 2.7.1.—) | | |
| | | (NIMA-RELATED | | |
| | | PROTEIN KINASE 2) | | |
| | | (NIMA-LIKE | | |
| | | PROTEIN KINASE 1) | | |
| | | (HSPK 21). | | |
| FACTYVGTPYY | NEK3_HUMAN | SERINE/THREONINE- | F158 | T161/ |
| VP | | PROTEIN KINASE | P170 | autophosphorylation |
| | | NEK3 (EC 2.7.1.—) | | |
| | | (NIMA-RELATED | | |
| | | PROTEIN KINASE 3) | | |
| | | (HSPK 36) | | |
| | | (FRAGMENT). | | |
| NHCDMASTLIG | NEK4_HUMAN | SERINE/THREONINE- | N158 | T165/ |
| TP | | PROTEIN KINASE | P170 | autophosphorylation |
| | | NRK2 (EC 2.7.1.—) | | |
| | | (SERINE/THREONINE | | |
| | | KINASE 2). | | |
| PTAGALYSGSE | B296-A | SERUM RESPONSE | P70 D82 | S77 S79 |
| GD | | FACTOR (SRF) | | |
| YSGSEGDSESGEE | B296-C | SERUM RESPONSE | Y76 E88 | S83/CK2 |
| | | FACTOR (SRF) | | |
| RGLKRSLSEMEIG | B296-E | SERUM RESPONSE | R96 | S103/ |
| | | FACTOR (SRF) | G108 | MK2, |
| | | | | pp90rsk, |
| | | | | CaMKII |
| ELFDDPSYVNV | A052-A | SHC | E420 | Y427/ |
| QN | | TRANSFORMING | N432 | TRK-T3, |
| | | PROTEINS 46.8 KD | | IR, MAP |
| | | AND 51.7 KD | | kinase, Fyn, |
| | | | | Lck, Grb2 |
| TDNLLPMSPEEFD | STA1_HUMAN | SIGNAL | T720 | S727/MAP |
| | | TRANSDUCER AND | D732 | kinase |
| | | ACTIVATOR OF | | family |
| | | TRANSCRIPTION 1- | | KIT, Akt |
| | | ALPHA/BETA | | |
| | | (TRANSCRIPTION | | |
| | | FACTOR | | |
| | | ISGF-3 | | |
| | | COMPONENTS | | |
| | | P91/P84). | | |
| DGPKGTGYIKT | STA1_HUMAM | SIGNAL | D694 | Y701/ |
| EL | | TRANSDUCER AND | L706 | JAK1, |
| | | ACTIVATOR OF | | JAK2, |
| | | TRANSCRIPTION 1- | | TYK2, |
| | | ALPHA/BETA | | EGFR, Lck |
| | | (TRANSCRIPTION | | |
| | | FACTOR ISGF-3 | | |
| | | COMPONENTS | | |
| | | P91/P84). | | |
| NTIDLPMSPRALD | STA3_HUMAN | SIGNAL | N720 | S727/PKC |
| | | TRANSDUCER AND | D732 | delta, JNK1, |
| | | ACTIVATOR OF | | ERK1, |
| | | TRANSCRIPTION 3 | | ERK2 |
| | | (ACUTE-PHASE | | |
| | | RESPONSE FACTOR). | | |
| DPGSAAPYLKT | STA3_HUMAN | SIGNAL | D698 | Y705/ |
| KF | | TRANSDUCER AND | F710 | JAK1, c-Src |
| | | ACTIVATOR OF | | |
| | | TRANSCRIPTION 3 | | |
| | | (ACUTE-PHASE | | |
| | | RESPONSE FACTOR). | | |
| PSDLLPMSPSVYA | STA4_HUMAN | SIGNAL | P714 | S721/IL- |
| | | TRANSDUCER AND | A726 | 12, |
| | | ACTIVATOR OF | | MAP2K6, |
| | | TRANSCRIPTION 4. | | MAPK14 |
| TERGDKGYVPS | STA4_HUMAN | SIGNAL | T686 | Y693/ |
| VF | | TRANSDUCER AND | F698 | JAK, |
| | | ACTIVATOR OF | | MAP2K6, |
| | | TRANSCRIPTION 4. | | STAT4, IL- |
| | | | | 12 |
| DSLDSRLSPPAGL | STA5_HUMAN | SIGNAL | D773 | S780/IL 2 |
| | | TRANSDUCER AND | L785 | |
| | | ACTIVATOR OF | | |
| | | TRANSCRIPTION 5A. | | |
| MGKDGRGYVP | STA6_HUMAN | SIGNAL | M634 | Y641/IL 4, |
| ATI | | TRANSDUCER AND | I646 | JAK |
| | | ACTIVATOR OF | | |
| | | TRANSCRIPTION 6 | | |
| | | (IL-4 STAT). | | |
| SKEKIKQSSSSEC | CIN7_HUMAN | SODIUM CHANNEL | S862 | S869/PKA |
| | | PROTEIN, CARDIAC | C874 | |
| | | AND SKELETAL | | |
| | | MUSCLE ALPHA- | | |
| | | SUBUNIT. | | |
| TQNVPKDTMDH | CIN7_HUMAN | SODIUM CHANNEL | T770 | T777/PKA |
| VN | | PROTEIN, CARDIAC | N782 | |
| | | AND SKELETAL | | |
| | | MUSCLE ALPHA- | | |
| | | SUBUNIT. | | |
| QIEMKKRSPISTD | CIN7_HUMAN | SODIUM CHANNEL | Q435 | S442/PKA |
| | | PROTEIN, CARDIAC | D447 | |
| | | AND SKELETAL | | |
| | | MUSCLE ALPHA- | | |
| | | SUBUNIT. | | |
| KNGCRRGSSLG | CIN7_HUMAN | SODIUM CHANNEL | K898 | S905/PKA |
| QI | | PROTEIN, CARDIAC | I910 | |
| | | AND SKELETAL | | |
| | | MUSCLE ALPHA- | | |
| | | SUBUNIT. | | |
| DQARKAVSMH | NAC1_HUMAN | SODIUM/CALCIUM | D385 | S392 |
| EVN | | EXCHANGER 1 | N397 | |
| | | (NA+/CA2+- | | |
| | | EXCHANGE PROTEIN | | |
| | | 1). | | |
| VLCLRKGSGAK | SSR5_HUMAN | SOMATOSTATIN | V318 | S325/PKA |
| DA | | RECEPTOR TYPE 5 | A330 | |
| | | (SS5R). | | |
| EYTKEDGSKRIGM | SPIH_HUMAN | SPINDLIN HOMOLOG | E189 | S196 |
| | | (PROTEIN DXF34). | M201 | |
| KELEKRASGQA | STN1_HUMAN | STATHMIN | K8 E20 | S15/ |
| FE | | (PHOSPHOPROTEIN | | Ca2+/calmodulin- |
| | | P19) (PP19) | | dependent |
| | | (ONCOPROTEIN 18) | | kinase-Gr, |
| | | (OP18) (LEUKEMIA- | | PKA |
| | | ASSOCIATED | | |
| | | PHOSPHOPROTEIN | | |
| | | P18) (PP17) | | |
| | | (PROSOLIN) | | |
| | | (METABLASTIN) | | |
| | | (PR22 PROTEIN). | | |
| SVPEFPLSPPKKK | STN1_HUMAN | STATHMIN | S30 K42 | S37/cdc2, |
| | | (PHOSPHOPROTEIN | | p34cdc2 |
| | | P19) (PP19) | | |
| | | (ONCOPROTEIN 18) | | |
| | | (OP18) (LEUKEMIA- | | |
| | | ASSOCIATED | | |
| | | PHOSPHOPROTEIN | | |
| | | P18) (PP17) | | |
| | | (PROSOLIN) | | |
| | | (METABLASTIN) | | |
| | | (PR22 PROTEIN). | | |
| QAFELILSPRSKE | STN1_HUMAN | STATHMIN | Q17 E29 | S24/cdc2, |
| | | (PHOSPHOPROTEIN | | MAPK |
| | | P19) (PP19) | | |
| | | (ONCOPROTEIN 18) | | |
| | | (OP18) (LEUKEMIA- | | |
| | | ASSOCIATED | | |
| | | PHOSPHOPROTEIN | | |
| | | P18) (PP17) | | |
| | | (PROSOLIN) | | |
| | | (METABLASTIN) | | |
| | | (PR22 PROTEIN). | | |
| LRAQRASSNVF | MLR5_HUMAN | SUPERFAST MYOSIN | L14 N26 | S21 |
| SN | | REGULATORY | | |
| | | LIGHT CHAIN 2 | | |
| | | (MYOSIN | | |
| | | REGULATORY | | |
| | | LIGHT CHAIN 5). | | |
| AGPTRQASQAG | SYN1_HUMAN | SYNAPSIN I (BRAIN | A598 | S605/ |
| PV | | PROTEIN 4.1). | V610 | CaMK2 |
| PQATRQTSVSGPA | SYN1_HUMAN | SYNAPSIN I (BRAIN | P561 | S568/ |
| | | PROTEIN 4.1). | A573 | CaMK2 |
| NYLRRRLSDSN | SYN1_HUMAN | SYNAPSIN I (BRAIN | N2 M14 | S9/ |
| FM | | PROTEIN 4.1). | | CaMK1, |
| | | | | PKA |
| ITKALGISYGRKK | B105-A | TAT PROTEIN | I39 K51 | S46/PKC |
| | | (TRANSACTIVATING | | |
| | | REGULATORY | | |
| | | PROTEIN) | | |
| GVRQSRASDKQ | B054-B | T-CELL SURFACE | G141 | S145; S148/ |
| TL | | GLYCOPROTEIN CD3 | L153 | PKC |
| | | GAMMA CHAIN (T3 | | |
| | | GAMMA CHAIN) | | |
| GQDGVRQSRAS | B054-A | T-CELL SURFACE | G138 | S145; S148/ |
| DK | | GLYCOPROTEIN CD3 | K150 | PKC |
| | | GAMMA CHAIN (T3 | | |
| | | GAMMA CHAIN) | | |
| KDKMAEAYSEI | A057-B | T-CELL SURFACE | K116 | Y123/Lck, |
| GM | | GLYCOPROTEIN CD3 | M128 | Fyn |
| | | ZETA CHAIN | | |
| STATKDTYDAL | CD3Z_HUMAN | T-CELL SURFACE | S146 | Y153 |
| HM | | GLYCOPROTEIN CD3 | M158 | |
| | | ZETA CHAIN (T- | | |
| | | CELL RECEPTOR T3 | | |
| | | ZETA CHAIN). | | |
| GQSWKENSPLN | TF_HUMAN | TISSUE FACTOR (TF) | G283 | S285/PKC; |
| VS | | (COAGULATION | S295 | S290/PKC |
| | | FACTOR | | |
| | | III(THROMBOPLASTIN) | | |
| | | (CD142 ANTIGEN) | | |
| VGLLKLASPELER | B006-B | TRANSCRIPTION | V66 | S73/ |
| | | FACTOR AP-1 | R78 | p34cdc2, |
| | | (PROTO-ONCOGENE | | JNK1, |
| | | C-JUN) | | MAPK8 |
| KNSDLLTSPDV | B006-A | TRANSCRIPTION | K56 L68 | S63/ |
| GL | | FACTOR AP-1 | | p34cdc2, |
| | | (PROTO-ONCOGENE | | JNK1, |
| | | C-JUN) | | MAPK8 |
| VPEMPGETPPLSP | B006-E | TRANSCRIPTION | V232 | T239/ |
| | | FACTOR AP-1 | P244 | GSK3; |
| | | (PROTO-ONCOGENE | | S243/ERT |
| | | C-JUN) | | PK, GSK3 |
| PGETPPLSPIDME | B006-C | TRANSCRIPTION | P236 | T239/ |
| | | FACTOR AP-1 | E248 | GSK3; |
| | | (PROTO-ONCOGENE | | S243/ERT |
| | | C-JUN) | | PK, GSK3 |
| LSPIDMESQERIK | B006-D | TRANSCRIPTION | L242 | S243/ERT |
| | | FACTOR AP-1 | K254 | PK, GSK3; |
| | | (PROTO-ONCOGENE | | S249/ |
| | | C-JUN) | | DNA-PK, |
| | | | | GSK3, CKII |
| GFIDQNLSPTKGN | TDP2_HUMAN | TRANSCRIPTION | G17 | S24/CDK2 |
| | | FACTOR DP-2 (E2F | N29 | |
| | | DIMERIZATION | | |
| | | PARTNER 2). | | |
| FPVSNTNSPTKIL | TDP2_HUMAN | TRANSCRIPTION | F35 L47 | S42/CDK2 |
| | | FACTOR DP-2 (E2F | | |
| | | DIMERIZATION | | |
| | | PARTNER 2). | | |
| PRASPAHSPRENG | TLE1_HUMAN | TRANSDUCIN-LIKE | P260 | S263/ |
| | | ENHANCER PROTEIN | G272 | CDC2; |
| | | 1 (ESG1). | | S267/ |
| | | | | CDC2 |
| DPSSPRASPAHSP | TLE1_HUMAN | TRANSDUCIN-LIKE | D256 | S259/ |
| | | ENHANCER PROTEIN | P268 | CDC2; |
| | | 1 (ESG1). | | S263/ |
| | | | | CDC2; |
| | | | | S267/ |
| | | | | CDC2 |
| VSNEDPSSPRASP | TLE1_HUMAN | TRANSDUCIN-LIKE | V252 | S259/ |
| | | ENHANCER PROTEIN | P264 | CDC2; |
| | | 1 (ESG1). | | S263/ |
| | | | | CDC2 |
| KDSSHYDSDGD | TLE1_HUMAN | TRANSDUCIN-LIKE | K232 | S239/CK2 |
| KS | | ENHANCER PROTEIN | S244 | |
| | | 1 (ESG1). | | |
| EPPSPATTPCGKV | TLE2_HUMAN | TRANSDUCIN-LIKE | E246 | S249/ |
| | | ENHANCER PROTEIN | V258 | CDC2; |
| | | 2 (ESG2). | | T253/ |
| | | | | CDC2 |
| EPSGPYESDEDKS | TLE2_HUMAN | TRANSDUCIN-LIKE | E221 | S228/CK2 |
| | | ENHANCER PROTEIN | S233 | |
| | | 2 (ESG2). | | |
| DQPSEPPSPATTP | TLE2_HUMAN | TRANSDUCIN-LIKE | D242 | S249/ |
| | | ENHANCER PROTEIN | P254 | CDC2; |
| | | 2 (ESG2). | | T253/ |
| | | | | CDC2 |
| DSLSRYDSDGD | TLE3_HUMAN | TRANSDUCIN-LIKE | D233 | S240/CK2 |
| KS | | ENHANCER PROTEIN | S245 | |
| | | 3 (ESG3). | | |
| PLSYTRFSLARQV | TFR1_HUMAN | TRANSFERRIN | P17 V29 | S24/PKC |
| | | RECEPTOR PROTEIN | | |
| | | (TR) (ANTIGEN CD71) | | |
| | | (T9) (P90). | | |
| SDTEEQEYEEEQP | TRT1_HUMAN | TROPONIN T, SLOW | S1 P13 | S1/CK2 |
| | | SKELETAL MUSCLE | | |
| | | ISOFORMS. | | |
| TASSGADYPDE | TRY1_HUMAN | TRYPSIN I (EC | T147 | Y154 |
| LQ | | 3.4.21.4) (CATIONIC | Q159 | |
| | | TRYPSINOGEN). | | |
| RKSKRRNSEFEIF | TPH1_HUMAN | TRYPTOPHAN 5- | R51 F63 | S58/PKA |
| | | MONOOXYGENASE | | |
| | | (EC 1.14.16.4) | | |
| | | (TRYPTOPHAN 5- | | |
| | | HYDROXYLASE). | | |
| RFIGRRQSLIEDA | TY3H_HUMAN | TYROSINE 3- | R64 | S71/PKA |
| | | MONOOXYGENASE | A76 | |
| | | (EC 1.14.16.2) | | |
| | | (TYROSINE | | |
| | | 3-HYDROXYLASE) | | |
| | | (TH). | | |
| ALRERLSSFTSYE | PTK6_HUMAN | TYROSINE-PROTEIN | A436 | Y447/ |
| | | KINASE 6 (EC | E448 | autophosphorylation |
| | | 2.7.1.112) (BREAST | | |
| | | TUMOR KINASE) | | |
| | | (TYROSINE-PROTEIN | | |
| | | KINASE BRK). | | |
| RLMTGDTYTAH | ABL2_HUMAN | TYROSINE-PROTEIN | R405 | Y412/auto, |
| AG | | KINASE ABL2 (EC | G417 | c-Src; T413 |
| | | 2.7.1.112) (TYROSINE | | |
| | | KINASE ARG). | | |
| ARIIDSEYTAQEG | BLK_HUMAN | TYROSINE-PROTEIN | A381 | Y388/ |
| | | KINASE BLK (EC | G393 | autophosphorylation |
| | | 2.7.1.112) (B | | |
| | | LYMPHOCYTE | | |
| | | KINASE) (P55-BLK). | | |
| KVDNEDIYESR | FRK_HUMAN | TYROSINE-PROTEIN | K380 | Y387/ |
| HE | | KINASE FRK (EC | E392 | autophosphorylation |
| | | 2.7.1.112) (NUCLEAR | | |
| | | TYROSINE PROTEIN | | |
| | | KINASE RAK). | | |
| AIETDKEYYTV | JAK1_HUMAN | TYROSINE-PROTEIN | A1015 | Y1022/ |
| KD | | KINASE JAK1 (EC | D1027 | JAK 3; |
| | | 2.7.1.112) (JANUS | | Y1023/ |
| | | KINASE 1) (JAK-1). | | JAK 3 |
| LSRGEEVYVKK | TIE1_HUMAN | TYROSINE-PROTEIN | L1000 | Y1007/ |
| TM | | KINASE RECEPTOR | M1012 | autophosphorylation |
| | | TIE-1 (EC | | |
| | | 2.7.1.112). | | |
| KIYSGDYYRQG | TYO3_HUMAN | TYROSINE-PROTEIN | K679 | Y686/ |
| CA | | KINASE RECEPTOR | A691 | autophosphorylation |
| | | TYRO3 (TYROSINE- | | |
| | | PROTEIN KINASE | | |
| | | RSE) (TYROSINE- | | |
| | | PROTEIN | | |
| | | KINASE SKY) | | |
| | | (TYROSINE-PROTEIN | | |
| | | KINASE DTK). | | |
| ALRADENYYKA | KSYK_HUMAN | TYROSINE-PROTEIN | A518 | Y525/ |
| QT | | KINASE SYK (EC | T530 | autophosphorylation; |
| | | 2.7.1.112) (SPLEEN | | Y526/ |
| | | TYROSINE KINASE). | | autophosphorylation |
| RYFLDDQYTSSSG | TEC_HUMAN | TYROSINE-PROTEIN | R512 | Y519/ |
| | | KINASE TEC (EC | G524 | autophosphorylation |
| | | 2.7.1.112). | | |
| ALGADDSYYTA | ZA70_HUMAN | TYROSINE-PROTEIN | A485 | Y492/auto, |
| RS | | KINASE ZAP-70 (EC | S497 | p56lck; |
| | | 2.7.1.112) (70 KDA | | Y493/ |
| | | ZETA-ASSOCIATED | | auto, p56lck |
| | | PROTEIN) (SYK- | | |
| | | RELATED TYROSINE | | |
| | | KINASE). | | |
| LGADDSYYTAR | ZA70_HUMAN | TYROSINE-PROTEIN | L486 | Y492/auto, |
| SA | | KINASE ZAP-70 (EC | A498 | p56lck; |
| | | 2.7.1.112) (70 KDA | | Y493/ |
| | | ZETA-ASSOCIATED | | auto, p56lck |
| | | PROTEIN) (SYK- | | |
| | | RELATED TYROSINE | | |
| | | KINASE). | | |
| TSMFDDYQGDS | VGR1_HUMAN | VASCULAR | T1236 | Y1242/ |
| ST | | ENDOTHELIAL | T1248 | autophosphorylation |
| | | GROWTH FACTOR | | |
| | | RECEPTOR 1 (EC | | |
| | | 2.7.1.112) (VEGFR-1) | | |
| | | (VASCULAR | | |
| | | PERMEABILITY | | |
| | | FACTOR RECEPTOR) | | |
| | | (TYROSINE-PROTEIN | | |
| | | KINASE | | |
| | | RECEPTOR FLT) | | |
| | | (FLT-1) | | |
| DIYKDPDYVRK | VGR3_HUMAN | VASCULAR | D1061 | Y1068/ |
| GS | | ENDOTHELIAL | S1073 | autophosphorylation |
| | | GROWTH FACTOR | | |
| | | RECEPTOR 3 (EC | | |
| | | 2.7.1.112) (VEGFR-3) | | |
| | | (TYROSINE-PROTEIN | | |
| | | KINASE | | |
| | | RECEPTOR FLT4). | | |
| GAKLRKVSKQE | VASP_HUMAN | VASODILATOR- | G231 | S238/ |
| EA | | STIMULATED | A243 | PKA, PKG |
| | | PHOSPHOPROTEIN | | |
| | | (VASP). | | |
| LARRRKATQVG | VASP_HUMAN | VASODILATOR- | L270 | T277/ |
| EK | | STIMULATED | K282 | PKA, PKG |
| | | PHOSPHOPROTEIN | | |
| | | (VASP). | | |
| EHIERRVSNAG | VASP_HUMAN | VASODILATOR- | E149 | S156/ |
| GP | | STIMULATED | P161 | PKA, PKG |
| | | PHOSPHOPROTEIN | | |
| | | (VASP). | | |
| YEEKKKKTTTIAV | VGLN_HUMAN | VIGILIN (HIGH | Y288 | T295 T296 |
| | | DENSITY | V300 | |
| | | LIPOPROTEIN | | |
| | | BINDING PROTEIN) | | |
| | | (HDL-BINDING | | |
| | | PROTEIN). | | |
| EEKKKKTTTIAVE | VGLN_HUMAN | VIGILIN (HIGH | E289 | T295 T296 |
| | | DENSITY | E301 | |
| | | LIPOPROTEIN | | |
| | | BINDING PROTEIN) | | |
| | | (HDL-BINDING | | |
| | | PROTEIN). | | |
| GVRLLQDSVDF | VIME_HUMAN | VIMENTIN. | G75 L87 | S82/ |
| SL | | | | CaMK2 |
| KSFLDSGYRILGA | VINC_HUMAN | VINCULIN. | K814 | Y821 |
| | | | A826 | |
| NQNSRRPSRAT | VTNC_HUMAN | VITRONECTIN | N390 | S397/PKA |
| WL | | (SERUM SPREADING | L402 | |
| | | FACTOR) (S- | | |
| | | PROTEIN) | | |
| | | [CONTAINS: | | |
| | | SOMATOMEDIN B]. | | |
| PKRGFLRSASLGR | CCAC_HUMAN | VOLTAGE- | P1966 | S1973/ |
| | | DEPENDENT L-TYPE | R1978 | PKA |
| | | CALCIUM CHANNEL | | |
| | | ALPHA- | | |
| | | 1C SUBUNIT | | |
| | | (CALCIUM | | |
| | | CHANNEL, L TYPE, | | |
| | | ALPHA-1 | | |
| | | POLYPEPTIDE, | | |
| | | ISOFORM 1, | | |
| | | CARDIAC MUSCLE). | | |
| ASLGRRASFHLEC | CCAC_HUMAN | VOLTAGE- | A1974 | S1981/ |
| | | DEPENDENT L-TYPE | C1986 | PKA |
| | | CALCIUM CHANNEL | | |
| | | ALPHA- | | |
| | | 1C SUBUNIT | | |
| | | (CALCIUM | | |
| | | CHANNEL, L TYPE, | | |
| | | ALPHA-1 | | |
| | | POLYPEPTIDE, | | |
| | | ISOFORM 1, | | |
| | | CARDIAC MUSCLE). | | |
| EKKRRKMSKGL | CCAS_HUMAN | VOLTAGE- | E680 | S687/PKA |
| PD | | DEPENDENT L-TYPE | D692 | |
| | | CALCIUM CHANNEL | | |
| | | ALPHA- | | |
| | | 1S SUBUNIT | | |
| | | (CALCIUM | | |
| | | CHANNEL, L TYPE, | | |
| | | ALPHA-1 | | |
| | | POLYPEPTIDE, | | |
| | | ISOFORM 3, | | |
| | | SKELETAL MUSCLE). | | |
| EYLTRDSSILGPH | CCAE_HUMAN | VOLTAGE- | E1726 | S1733/ |
| | | DEPENDENT R-TYPE | H1738 | PKA |
| | | CALCIUM CHANNEL | | |
| | | ALPHA-1E SUBUNIT | | |
| | | (CALCIUM | | |
| | | CHANNEL, L TYPE, | | |
| | | ALPHA-1 | | |
| | | POLYPEPTIDE, | | |
| | | ISOFORM 6) (BRAIN | | |
| | | CALCIUM CHANNEL | | |
| | | II) (BII). | | |
| DSDLSRRSSSTMS | CIK1_HUMAN | VOLTAGE-GATED | D438 | S445/PKA |
| | | POTASSIUM | S450 | |
| | | CHANNEL PROTEIN | | |
| | | KV1.1 (HUKI) (HBK1). | | |
| LGQTLKASMRE | CIK1_HUMAN | VOLTAGE-GATED | L315 | S322/PKA |
| LG | | POTASSIUM | G327 | |
| | | CHANNEL PROTEIN | | |
| | | KV1.1 (HUKI) (HBK1). | | |
| PDLKKSRSASTIS | CIK2_HUMAN | VOLTAGE-GATED | P442 | S449/PKA |
| | | POTASSIUM | S454 | |
| | | CHANNEL PROTEIN | | |
| | | KV1.2 (RBK2) (HBK5) | | |
| | | (NGK1) (MK2) | | |
| | | (HUKIV). | | |
| EELRKARSNSTLS | CIK3_HUMAN | VOLTAGE-GATED | E461 | S468/PKA |
| | | POTASSIUM | S473 | |
| | | CHANNEL PROTEIN | | |
| | | KV1.3 (HPCN3) | | |
| | | (HGK5) (HUKIII) | | |
| | | (HLK3). | | |
| REEEATRSEKK | CIK4_HUMAN | VOLTAGE-GATED | R83 | S90/PKA |
| KA | | POTASSIUM | A95 | |
| | | CHANNEL PROTEIN | | |
| | | KV1.4 (HK1) (HPCN2) | | |
| | | (HBK4) (HUKII). | | |
| NLLKKFRSSTSSS | CIK4_HUMAN | VOLTAGE-GATED | N592 | S599/PKA |
| | | POTASSIUM | S604 | |
| | | CHANNEL PROTEIN | | |
| | | KV1.4 (HK1) (HPCN2) | | |
| | | (HBK4) (HUKII). | | |
| RGVQRKVSGSR | CIK5_HUMAN | VOLTAGE-GATED | R550 | S557/PKA |
| GS | | POTASSIUM | S562 | |
| | | CHANNEL PROTEIN | | |
| | | KV1.5 | | |
| | | (HK2) (HPCN1). | | |
| ANRERRPSYLPTP | CIK6_HUMAN | VOLTAGE-GATED | A504 | S511/PKA |
| | | POTASSIUM | P516 | |
| | | CHANNEL PROTEIN | | |
| | | KV1.6 (HBK2). | | |
| KWTKRTLSETS | KCB1_HUMAN | VOLTAGE-GATED | K489 | |
| SS | | POTASSIUM | S501 | |
| | | CHANNEL PROTEIN | | |
| | | KV2.1 (DHK1). | | |
| EEGFGSSSPVKSP | WEE1_HUMAN | WEE1-LIKE PROTEIN | E116 | S123/cdc2 |
| | | KINASE (EC | P128 | |
| | | 2.7.1.112). | | |
| YFLGSSFSPVRCG | WEE1_HUMAN | WEE1-LIKE PROTEIN | Y132 | S139/cdc2 |
| | | KINASE (EC | G144 | |
| | | 2.7.1.112). | | |
| SFGLSAMSPTK | Z145_HUMAN | ZINC FINGER | S190 | S197/ |
| AA | | PROTEIN PLZF | A202 | PDPK |
| | | (PROMYELOCYTIC | | |
| | | LEUKEMIA ZINC | | |
| | | FINGER PROTEIN) | | |
| | | (ZINC FINGER | | |
| | | PROTEIN 145). | | |
| HYTLDFLSPKTFQ | Z145_HUMAN | ZINC FINGER | H69 | S76/PDPK |
| | | PROTEIN PLZF | Q81 | |
| | | (PROMYELOCYTIC | | |
| | | LEUKEMIA ZINC | | |
| | | FINGER PROTEIN) | | |
| | | (ZINC FINGER | | |
| | | PROTEIN 145). | | |
| RGKEGPGTPTRSS | Z145_HUMAN | ZINC FINGER | R275 | T282/ |
| | | PROTEIN PLZF | S287 | PDPK |
| | | (PROMYELOCYTIC | | |
| | | LEUKEMIA ZINC | | |
| | | FINGER PROTEIN) | | |
| | | (ZINC FINGER | | |
| | | PROTEIN 145). | | |
| DEVPSQDSPGA | Z145_HUMAN | ZINC FINGER | D249 | S256/ |
| AE | | PROTEIN PLZF | E261 | PDPK |
| | | (PROMYELOCYTIC | | |
| | | LEUKEMIA ZINC | | |
| | | FINGER PROTEIN) | | |
| | | (ZINC FINGER | | |
| | | PROTEIN 145). | | |
| PGPMVDQSPSV | Z145_HUMAN | ZINC FINGER | P177 | S184/ |
| ST | | PROTEIN PLZF | T189 | PDPK |
| | | (PROMYELOCYTIC | | |
| | | LEUKEMIA ZINC | | |
| | | FINGER PROTEIN) | | |
| | | (ZINC FINGER | | |
| | | PROTEIN 145). | | |
| LRTHNGASPYQ | Z145_HUMAN | ZINC FINGER | L621 | S628/ |
| CT | | PROTEIN PLZF | T633 | PDPK |
| | | (PROMYELOCYTIC | | |
| | | LEUKEMIA ZINC | | |
| | | FINGER PROTEIN) | | |
| | | (ZINC FINGER | | |
| | | PROTEIN 145). | | |
| KEKPDSSLPETSK | XRC4_HUMAN | DNA-repair protein | K296 | |
| | (Q13426-2) | XRCC4, Splice isoform | K308 | |
| | | 2 (X-ray rep air cross- | | |
| | | complementing protein | | |
| | | 4) | | |
| VLLLQDSSGDY | KPCM_HUMAN | Protein kinase C, mu | V62 L74 | |
| SL | | type (EC 2.7.1.—) | | |
| | | (nPKC-mu) (Protein | | |
| | | kinase | | |
| | | D) | | |
| SQGRNCSTNDS | B204-B | beta-2 adrenergic | S401 | S401/ |
| LL | | receptor | L413 | GRK2, |
| | | | | GRK5; |
| | | | | S407/ |
| | | | | GRK2, |
| | | | | GRK5; |
| | | | | S411/ |
| | | | | GRK5 |
| SVEPPLSQETFSD | P53_HUMAN | Cellular tumor antigen | S9 D21 | S15/ |
| | | p53 (Tumor suppressor | | PRPK; T18/ |
| | | p53) (Phosphoprotein | | VRK1, |
| | | p53) (Antigen NY-CO- | | CK1 delta; |
| | | 13) | | S20/Plk3, |
| | | | | Chk2 |
| GQLVDSIAKTR | MPK4_HUMAN | Dual specificity | G252 | S257/ |
| DA | | mitogen-activated | A264 | MAP2K4; |
| | | protein kinase kinase 4 | | T261/ |
| | | (EC | | MAP2K4 |
| | | 2.7.1.—) (MAP kinase | | |
| | | kinase 4) (JNK | | |
| | | activating kinase 1) (c- | | |
| | | Jun N-terminal kinase | | |
| | | kinase 1) (JNKK) | | |
| | | (SAPK/ERK kinase 1) | | |
| | | (SEK1) | | |
| GYLVDSVAKTI | MPK6_HUMAN | Dual specificity | G202 | S207 T211 |
| DA | | mitogen-activated | A214 | |
| | | protein kinase kinase 6 | | |
| | | (EC | | |
| | | 2.7.1.—) (MAP kinase | | |
| | | kinase 6) (MAPKK 6) | | |
| | | (MAPK/ERK kinase 6) | | |
| | | (SAPKK3) | | |
| YGNGYSSNGNT | B2AR_HUMAN | Beta-2 adrenergic | Y350 | Y350/IR; |
| GE | | receptor | E362 | Y354/IR; |
| | | | | S355/ |
| | | | | beta ARK; |
| | | | | S356/beta |
| | | | | ARK |
| IYDALSYSSPSDS | PVR2_HUMAN | I512 S524 | | |
| | Poliovirus | | | |
| | receptor | | | |
| | related protein | | | |
| | 2 precursor | | | |
| | (Herpes virus | | | |
| | entry mediator | | | |
| | B) (HveB) | | | |
| | (Nectin 2) | | | |
| | (CD112 | | | |
| | antigen) | | | |
| DSDESSDDDSDSE | CD45_HUMAN | Leukocyte common | D998 | S999/CK2 |
| | | antigen precursor (EC | E1010 | alpha 1, CK |
| | | 3.1.3.48) (L-CA) (CD45 | | 2 alpha 2; |
| | | antigen) (T200) | | S1002/ |
| | | | | CK2 alpha |
| | | | | 1, CK 2 |
| | | | | alpha 2; |
| | | | | S1003/ |
| | | | | CK2 alpha |
| | | | | 1, CK 2 |
| | | | | alpha 2; |
| | | | | S1007/ |
| | | | | PKC |
| LRPSFPNRWSSDE | BMRB_HUMAN | Bone morphogenetic | L449 | |
| | | protein receptor type IB | E461 | |
| | | precursor (EC | | |
| | | 2.7.1.37) | | |
| GFDRLSTEGSD | AMD_HUMAN | Peptidyl-glycine alpha- | G937 | S946/P- |
| QE | | amidating | E949 | CIP2 |
| | | monooxygenase | | |
| | | precursor (EC | | |
| | | 1.14.17.3) (PAM) | | |
| ESTTSDSDQNFDY | CAD8_HUMAN | Cadherin-8 precursor | E763 | |
| | | | Y775 | |
| GEYRSLESDNEEK | CAML_HUMAN | Neural cell adhesion | G1174 | |
| | | molecule L1 precursor | K1186 | |
| | | (N-CAM L1) (CD171 | | |
| | | antigen) | | |
| SSQTSQDSGNYSN | INR1_HUMAN | Interferon-alpha/beta | S528 | |
| | | receptor alpha chain | N540 | |
| | | precursor (IFN-alpha- | | |
| | | REC) | | |
| ESLSSLGTDSSDS | CAD5_HUMAN | Vascular endothelial- | E741 | |
| | | cadherin precursor (VE- | S753 | |
| | | cadherin) (Cadherin- | | |
| | | 5) (7B4 antigen) | | |
| | | (CD144 antigen) | | |
| KMLKKRSPLTT | CTL4_HUMAN | Cytotoxic T-lymphocyte | K188 | |
| GV | | protein 4 precursor | V200 | |
| | | (Cytotoxic T- | | |
| | | lymphocyte-associated | | |
| | | antigen 4) (CTLA-4) | | |
| | | (CD152 antigen) | | |
| RRKRKPSTSDD | TP2A_HUMAN | DNA topoisomerase II, | R1463 | S1469/ |
| SD | | alpha isozyme (EC | D1475 | CK2 |
| | | 5.99.1.3) | | |
| SVYESPYSDPEEL | ZA70_HUMAN | Tyrosine-protein kinase | S313 | Y315/ |
| | | ZAP-70 (EC 2.7.1.112) | L325 | autophosphorylation; |
| | | (70 kDa zeta-associated | | Y319/ |
| | | protein) (Syk-related | | autophosphorylation |
| | | tyrosine kinase) | | |
| EYQGDQSDTED | YL1_HUMAN | YL-1 protein | E45 V57 | S51/CK2 |
| EV | | (Transcription factor- | | |
| | | like 1) | | |
| TAGALYSGSEG | B296-A | serum response factor | T71 S83 | S77 S79; |
| DS | | (SRF) | | S83/CK2 |
| GGFTEESGDDE | YL1_HUMAN | YL-1 protein | G35 | S41/CK2 |
| YQ | | (Transcription factor- | Q47 | |
| | | like 1) | | |
| GALYSGSEGDS | B296-A | serum response factor | G73 S85 | S77 S79; |
| ES | | (SRF) | | S83/CK2; |
| | | | | S85 |
| SGSEGDSESGEEE | B296-A | serum response factor | S77 E89 | S77 S79; |
| | | (SRF) | | S83/CK2; |
| | | | | S85 |
| IKVEPASPPYYSE | PPAT_HUMAN | Peroxisome proliferator | I106 | S112/ |
| | | activated receptor | E118 | ERK2, JNK |
| | | gamma (PPAR-gamma) | | |
| PDHYRYSDTTD | PTEN_HUMAN | Phosphatidylinositol- | P374 | S380/PKB; |
| SD | | 3,4,5-trisphosphate 3- | D386 | T382/CK2; |
| | | phosphatase PTEN (EC | | T383/CK2; |
| | | 3.1.3.67) (Mutated in | | S385/CK2, |
| | | multiple advanced | | PKB |
| | | cancers 1) | | |
| VEEEDSSESEESA | | | | |
| RSSLKAYGNGY | B2AR_HUMAN | Beta-2 adrenergic | R344 | S345/PKC, |
| SS | | receptor | S356 | PKA; S346/ |
| | | | | PKC, |
| | | | | PKA; Y350/ |
| | | | | IR; Y354/ |
| | | | | IR; S355/ |
| | | | | beta ARK; |
| | | | | S356/ |
| | | | | beta ARK |
| SGEDTLSDSDDED | | | | |
| KAYGNGYSSNG | B2AR_HUMAN | Beta-2 adrenergic | K348 | Y350/IR; |
| NT | | receptor | T360 | Y354/IR; |
| | | | | S355/ |
| | | | | beta ARK; |
| | | | | S356/beta |
| | | | | ARK |
| DSSHYDSDGDK | TLE1_HUMAN | Transducin-like | D233 | S239/CK2 |
| SD | | enhancer protein 1 | D245 | |
| | | (ESG1) | | |
| SLSRYDSDGDK | TLE3_HUMAN | Transducin-like | S234 | S240/CK2 |
| SD | | enhancer protein 3 | D246 | |
| | | (ESG3) | | |
| YFLDDQYTSSS | TEC_HUMAN | Tyrosine-protein kinase | Y513 | Y519/ |
| GA | | Tec (EC 2.7.1.112) | A525 | autophosphorylation |
| LSADEDSPSSPED | NRF1_HUMAN | Nuclear respiratory | L38 D50 | S39/CK2; |
| | | factor-1 (NRF-1) (Alpha | | S44/CK2; |
| | | palindromic binding | | S46/CK2; |
| | | protein) (Alpha-pal) | | S47/CK2 |
| DKDGNGYISAA | B227-A | Calmodulin | D93 | Y99/INSR; |
| EL | | | L105 | S101/CK2 |
| LKEQTGSDDED | DCOR_HUMAN | Ornithine decarboxylase | L297 | S303/CK2 |
| ES | | (EC 4.1.1.17) (ODC) | S309 | |
| PSGPYESDEDKSD | TLE2_HUMAN | Transducin-like | P222 | S228/CK2 |
| | | enhancer protein 2 | D234 | |
| | | (ESG2) | | |
| PLNVSFTNRNY | MYC_HUMAN | Myc proto-oncogene | P2 L14 | T8/c-RAF |
| DL | | protein (c-myc) | | |
| QRHLDISRELNDK | LGN_HUMAN | Mosaic protein LGN | Q119 | S125/PKG |
| | | | K131 | |
| EESQYDSGIESLR | IKBE_HUMAN | NF-kappaB inhibitor | E12 R24 | S18 S22 |
| | | epsilon (NF-kappa-BIE) | | |
| | | (I-kappa-B-epsilon) | | |
| | | (IkappaBepsilon) (IKB- | | |
| | | epsilon) (IKBE) | | |
| SAASFEYTILDPS | EPOR_HUMAN | Erythropoietin receptor | S420 | Y426/Jak2 |
| | | precursor (EPO-R) | S432 | |
| MGPRAASPMNH | CBP_HUMAN | CREB-binding protein | M739 | |
| SV | | (EC 2.3.1.48) | V751 | |
| FHLMAPSEEDH | IBP1_HUMAN | Insulin-like growth | F138 | S144 |
| SI | | factor binding protein 1 | I150 | |
| | | precursor (IGFBP-1) | | |
| | | (IBP-1) (IGF-binding | | |
| | | protein 1) (Placental | | |
| | | protein 12) (PP12) | | |
| AFLLESTMNEY | GLK4_HUMAN | Glutamate receptor, | A719 | |
| YR | | ionotropic kainate 4 | R731 | |
| | | precursor (Glutamate | | |
| | | receptor KA-1) (KA1) | | |
| | | (Excitatory amino acid | | |
| | | receptor 1) (EAA1) | | |
| SQGRNCSTNDS | B204-B | Beta-2 adrenergic | S401 | S401/ |
| LL | | receptor | L413 | GRK2, |
| | | | | GRK5; |
| | | | | S407/ |
| | | | | GRK2, |
| | | | | GRK5; |
| | | | | S411/ |
| | | | | GRK5 |
| FPQKIMTPADLSI | PIA1_HUMAN | Protein inhibitor of | F65 I77 | |
| | | activated STAT protein | | |
| | | 1 (Gu binding protein) | | |
| | | (GBP) (RNA helicase II | | |
| | | binding protein) | | |
| | | (DEAD/H box-binding | | |
| | | protein 1) | | |
| YLPLSLDDSDSLG | DCX_HUMAN | Doublecortin | Y387 | S391/CK2; |
| | | (Lissencephalin-X) (Lis- | G399 | S397/CK2 |
| | | X) (Doublin) | | |
| LADLTRSLSDNIN | DCX_HUMAN | Doublecortin | L126 | S132/CK2 |
| | | (Lissencephalin-X) (Lis- | N138 | |
| | | X) (Doublin) | | |
| LTSNQEYLDLS | FGR1_HUMAN | Basic fibroblast growth | L760 | Y766/ |
| MP | | factor receptor 1 | P772 | FGFR1 |
| | | precursor (EC 2.7.1.112) | | |
| | | (FGFR-1) (bFGF-R) | | |
| | | (Fms-like tyrosine | | |
| | | kinase-2) (c-fgr) | | |

What is claimed is:

1. A substrate for phosphorylation by a protein kinase comprising:
a core peptide having the Formula (I): (N-terminus)-Arginine-Lysine-Arginine-Serine-Arginine-Lysine-Glutamic-acid-(C-terminus) and
an indicator component covalently bonded to said core peptide.

2. The substrate of claim 1 wherein said substrate further comprises a linker covalently bonded to and disposed between each of said core peptide and said indicator component.

3. The substrate of claim 2 wherein said linker is directly covalently bonded to said C-terminus of said core peptide and to said indicator component.

4. The substrate of claim 2 wherein said linker is indirectly covalently bonded to said C-terminus of said core peptide and to said indicator component.

5. The substrate of claim 2 wherein said linker is directly covalently bonded to said C-terminus of said core peptide and indirectly covalently bonded to said indicator component.

6. The substrate of claim 2 wherein said linker is indirectly covalently bonded to said C-terminus of said core peptide and directly covalently bonded to said indicator component.

7. The substrate of claim wherein said linker comprises a linear aminoalkanoic acid having at least six carbon atoms for spacing said indicator component from said core peptide.

8. The substrate of claim 7 wherein said linear aminoalkanoic acid is 6-aminohexanoic acid.

9. The substrate of claim 1 wherein said indicator component comprises a fluorophore.

10. The substrate of claim 1 wherein said indicator component is excited by light at a wavelength of from about 700 nm to about 1000 nm and subsequently emits light at a wavelength of from about 700 nm to about 1000 nm.

11. The substrate of claim 2 further comprising an amino acid anchor covalently bonded to and disposed between each of said linker and said indicator component.

12. The substrate of claim 11 wherein said amino acid anchor is further defined as cysteine.

13. The substrate of claim 1 wherein said protein kinase is sufficiently capable of phosphorylating said core peptide to form a phosphorylated substrate.

14. The substrate of claim 11 wherein said protein kinase is a protein kinase C, a protein kinase G, or a combination thereof.

15. The substrate of claim 12 wherein only said protein kinase C and protein kinase G are sufficiently capable of phosphorylating said core peptide to form said phosphorylated substrate.

16. A method for detecting a phosphorylated substrate, said method comprising the steps of:
providing a non-phosphorylated substrate comprising a core peptide;
phosphorylating the core peptide with a protein kinase to form the phosphorylated substrate; and
detecting the phosphorylated substrate;
wherein the non-phosphorylated substrate comprises;
a core peptide having the Formula (I): (N-terminus)-Arginine-Lysine-Arginine-Serine-Arginine-Lysine-Glutamic-acid-(C-terminus) and
an indicator component covalently bonded to the core peptide.

17. The method of claim 16 further comprising the steps of:
combining the non-phosphorylated substrate and the protein kinase to form a reaction mixture;
incubating the reaction mixture for twenty minutes or less at about 30° C.;
combining the reaction mixture and EDTA to terminate the phosphorylation of the non-phosphorylated substrate by the protein kinase; and
separating the phosphorylated substrate from the non-phosphorylated substrate.

18. The method of claim 17 wherein the step of separating is further defined as separating the phosphorylated substrate from the non-phosphorylated substrate by electrophoresis wherein the phosphorylated substrate has a negative charge, and the non-phosphorylated substrate has a positive charge, each in the presence of a buffer composition.

19. The method for claim 16 wherein the step of detecting the phosphorylated substrate comprises the steps of:
exciting the indicator component of the phosphorylated substrate with light at a wavelength of from about 700 nm to about 1000 nm such that the indicator component emits light at a wavelength of from about 700 nm to about 1000 nm; and
quantifying the light emitted.

20. A substrate for phosphorylation by a protein kinase comprising:
a core peptide having the Formula (I): (N-terminus)-Arginine-Lysine-Arginine-Serine-Arginine-Lysine-Glutamic-acid-(C-terminus);
a linker comprising 6-aminohexanoic acid and directly covalently bonded to said C-terminus of said core peptide;
an amino acid anchor comprising cysteine and directly covalently bonded to said linker; and
an indicator component comprising a fluorophore and directly covalently bonded to said amino acid anchor;
wherein said indicator component is excited by light at a wavelength of from about 700 nm to about 1000 nm and subsequently emits light at a wavelength of from about 700 nm to about 1000 nm;
wherein said protein kinase is protein kinase C, protein kinase G, or a combination thereof; and
wherein only said protein kinase C and protein kinase G are sufficiently capable of phosphorylating said core peptide to form said phosphorylated substrate.
